(19) United States
(12) Patent Application Publication
(10) Pub. No.: US 2006/0235037 A1
(43) Pub. Date: Oct. 19, 2006

(54) Heterocyclic inhibitors of protein arginine methyl transferases

(76) Inventors: Ashok Vinayak Purandare, Pennington, NJ (US); Honghe Wan, Pennington, NJ (US); Tram N. Huynh, Pennington, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/403,790

(22) Filed: Apr. 13, 2006

Related U.S. Application Data
(60) Provisional application No. 60/671,995, filed on Apr. 15, 2005.

(51) Int. Cl. A61K 31/4747; A61K 31/46; A61K 31/454; A61K 31/4184; A61K 31/404; C07D 403/02

(52) U.S. Cl. 514/278; 514/304; 514/314; 514/337; 514/338; 546/15; 546/125; 546/176; 546/201; 514/394; 548/305.4; 548/465; 546/199

(57) ABSTRACT

A compound of formula I, or a stereoisomer, a tautomer, a pharmaceutically acceptable salt or solvate thereof, 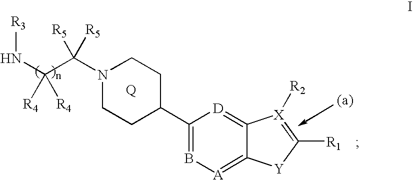 methods of using such compounds in the treatment of hyperproliferative, inflammatory, infectious, and immunoregulatory disorders and diseases; and to pharmaceutical compositions containing such compounds.

### RELATED APPLICATION

This application claims priority benefit under Title 35 § 119(e) of U.S. provisional Application No. 60/671,995, filed Apr. 15, 2005, the contents of which are herein incorporated by reference.

### FIELD OF THE INVENTION

The present invention relates to novel compounds which are inhibitors of Protein ARginine Methyl Transferases (PRMTs), to methods of using such compounds for inhibiting protein methyl transferases in the treatment of hyperproliferative, inflammatory, infectious, and immunoregulatory disorders and diseases, and to pharmaceutical compositions containing such compounds.

The invention also encompasses pharmaceutical compositions containing these compounds. The compounds and pharmaceutical compositions of the invention are particularly well suited as inhibitors of protein methyl transferases and, consequently, can be advantageously used as therapeutic agents for the treatment of, including cancer, asthma, COPD, and allergic diseases; rheumatoid arthritis, atherosclerosis, and psoriasis; solid organ transplant rejection, osteoarthritis, and inflammatory bowel syndrome. This invention also relates to methods of using the compounds of this invention alone or in combination with other pharmaceutically active agents.

### BACKGROUND OF THE INVENTION

PRMTs are enzymes that catalyze the transfer of methyl groups from S-Adenosyl-L-Methionine (SAM) to specific arginine residues of proteins. Arginine methylation of proteins has been implicated to play roles in pre-mRNA splicing, nucleo-cytoplasmic RNA transport, signal transduction and transcriptional activation. To date seven family members have been identified (PRMTs 1-7) in mammalian cells and they each appear to have distinct substrate preferences. PRMT1 has been shown to methyl ate Histone H4 and this results in activation of transcription. Coactivator Associated Argnine Methyltransferase-I (CARM-1) (also called PRMT-4) has been shown to methylate Histone H3 both in vitro and in vivo and it is speculated that this modification positively affects chromatin remodeling and thus transcriptional activation. CARM-1 has also been shown to play a co-activator role in gene transcription mediated by the nuclear hormone receptor (NHR) family of transcription factors. In this context, CARM-1 displays an absolute requirement for the presence a member of the NHR co-activator family of proteins (SRC-1, GRIP-1 or AIB1) in order to enhance transcriptional activation by the androgen receptor (AR) or estrogen receptor (ER). It is also able to co-operate with p300/CBP and PRMT-1 in NHR dependent transcription. In the resulting transcriptionally active complex, CARM-1 can methylate both p300/CBP and the NHR co-activator(s). The fact that mutations of critical residues in the catalytic domain compromise the co-activator function of CARM-1 suggests that the integrity of its methyl transferase domain is not dispensable for its co-activator function. Additionally, CARM-1 has been implicated to play a key role in mediating signal transduction pathways among CREB Binding Protein (CBP), p53 and the muscle enhancer factor −1 (MEF-1).

In addition to its role as a coactivator in NHR mediated signaling, CARM-1 has also been implicated as the PRMT responsible for methylation of PABP (Poly-A-binding protein) and HuR (a member of the Hu family of protein). PABP and HuR have been shown to bind to messenger RNAs (e.g. fos, Cox-2, β-catenin) containing the AUUUUA sequence in the 3′ untranslated region thereby stabilizing the message and leading to increased translation in the cytoplasm. Moreover, CARM1 has recently been implicated to have a regulatory role in the NF-κB signaling pathway (See Hottiger et. al, EMBO Journal; 24, 85, 2005).

Accordingly, CARM-1 may serve as an oncogene because its ability to regulate transcription, modulate chromatin organization and increase the half-life of specific mRNAs. Upon comparison of a number of matched tumor and normal tissues (lung, colon and breast), we found that CARM-1 was over-expressed in the tumor specimens compared to normal tissue (WO03102143). Moreover, Hong et al (Cancer, 101 (1), 83-89 (2004)) reports that CARM-1 levels are significantly higher in PIN (Prostatic intraepithelial neoplasia) and prostatic adenocarcinoma specimens from patients compared to benign prostate tissue specimens. See “Aberrant expression of CARM-1, a transcriptional coactivator of Androgen receptor in the development of prostate carcinoma and androgen independent status. In twelve patients with androgen independent prostatic adenocarcinoma, the expression of CARM-1 was significantly increased when compared to patients without previous hormonal treatment.

Furthermore, HuR, one of the substrates of CARM-1, has also been implicated in cancer (Li et al, J Biol Chem, 277, 44623 (2002), Erkinheimo et al, Cancer Res, 63, 7591 (2003)) HuR is a nuclear protein but is predominantly cytoplasmic in tumor cells. Increased cytoplasmic presence predicts a poor prognosis. Thus, methylation by CARM-1 may be responsible for the cytoplasmic presence of HuR which resulted in the increased stabilization of mRNAs of genes implicated in cancer (e.g. fos, Cox-2, β-catenin) (Denkert et al, Cancer Res, 64, 189 (2004). Accordingly, it is an attractive therapeutic option for cancer patients to inhibit the enzymatic function of CARM-1 by using a small organic molecule.

### SUMMARY OF THE INVENTION

The present invention provides a compound of the following formula I and/or Id, or a stereoisomer, a tautomer, a pharmaceutically acceptable salt or solvate thereof, which compounds are especially useful as inhibitors of PRMTs.

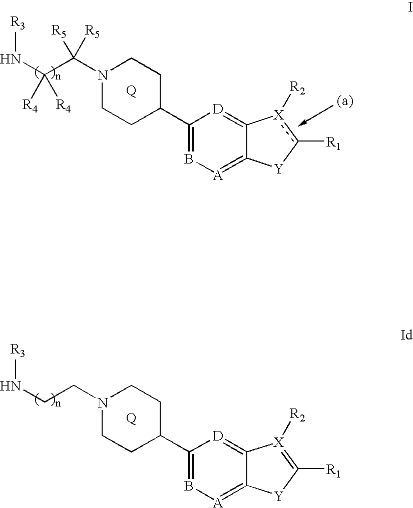

As used in formula I, Ia, Ib and Id, and throughout the specification, the symbols have the following meanings unless otherwise indicated, and are, for each occurrence, independently selected:
Ring Q 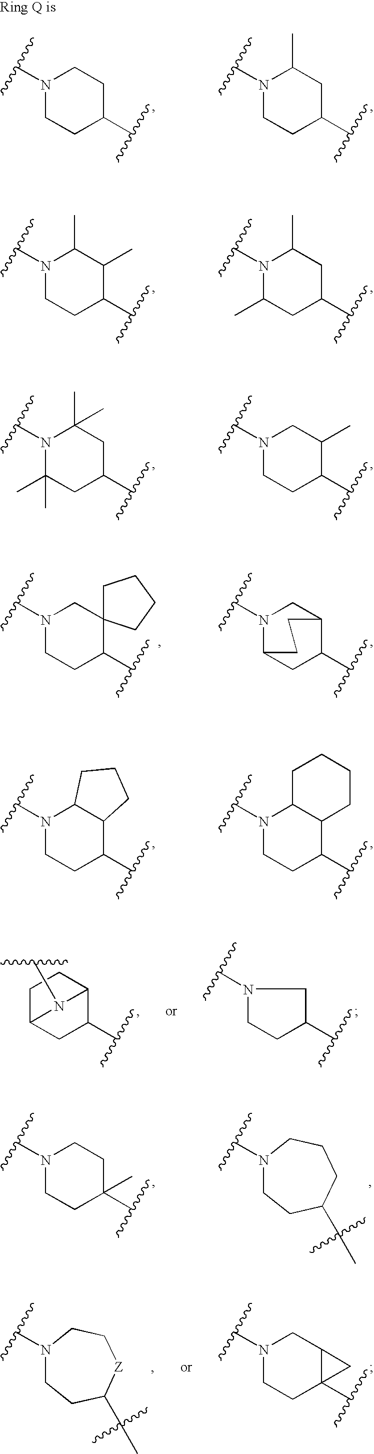
bond (a) is an optional double or single bond;
X is C (i.e., carbon) or N (i.e., nitrogen);
Y is NH, N-Me, or CH;
Z is N—R₆, O, or S, where R6 is C₁-C₆ alkyl;
wherein when bond (a) is a single bond, X is —CR—, R is indenpendently H or C₁₋₄ alkyl and CR₂ is H or C₁₋₄ alkyl; alternatively, R₂ and R may join to form a 3-6 membered cycloalkyl ring;
A, B and D are each independently N or C, in which C may be optionally substituted with H, Me, Et, halogen, CN, NO₂, OMe, OEt, SMe, SO₂Me, CF₃, or OCF₃;
R₁ is aryl, substituted aryl, heterocycle, or substituted heterocycle;
R₂ is H, Me, Et, halogen, CN, NO₂, OMe, OEt, SMe, SO₂Me, CF₃, or OCF₃, provided that when X is N, R₂is nil;
R₃ is H or C₁-C₄ alkyl; and
R₄ is independently H or C₁₋₄ alkyl;
R₅ is independently H, C₁₋₄ alkyl; alternatively, R5 and R3 may join to form a 4, 5, or 6 membered saturated ring containing one N; and
n is 1, 2, or 3.

The present invention also relates to methods of using compounds of formula I in the treatment of hyperproliferative, inflammatory, infectious, and immunoregulatory disorders and diseases, and to pharmaceutical compositions containing such compounds.

### FURTHER DESCRIPTION OF EMBODIMENTS OF THE INVENTION

### Definitions

The following are definitions of terms used in the present specification. The initial definition provided for a group or term herein applies to that group or term throughout the present specification individually or as part of another group, unless otherwise indicated.

The terms “alkyl” and “alk” refers to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “C₁-C₄ alkyl” refers to a straight or branched chain alkane (hydrocarbon) radical containing from 1 to 4 carbon atoms, such as methyl, ethyl, propyl, isopropyl, n-butyl, t-butyl, and isobutyl. “Substituted alkyl” refers to an alkyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein Rₐ is hydrogen, alkyl, alkyl substituted with halo, or NR_{b}R_{c}, haloalkyl, cycloalkyl, alkenyl, cycloalkenyl, alkynyl, heterocycle, or aryl or aryl substituted with halo, alkyl, O-alkyl, or haloalyl; R_{b}, R_{c} and R_{d} are independently hydrogen, alkyl, cycloalkyl, heterocycle, aryl, or said R_{b} and R_{c} together with the N to which they are bonded optionally form a heterocycle; and Rₑ is alkyl, cycloalkyl, alkenyl, cycloalkenyl, alkynyl, heterocycle, or aryl. In the aforementioned exemplary substitutents, groups such as alkyl, cycloalkyl, alkenyl, alkynyl, cycloalkenyl, heterocycle and aryl can themselves be optionally substituted.

The term “alkenyl” refers to a straight or branched chain hydrocarbon radical containing from 2 to 12 carbon atoms and at least one carbon-carbon double bond. Exemplary such groups include ethenyl or allyl. “Substituted alkenyl” refers to an alkenyl group substituted with one or more substituents, preferably 1 to 4 substituents, at any available point of attachment. Exemplary substituents include, but are not limited to, alkyl or substituted alkyl, as well as those groups recited above as exemplary alkyl substituents.

The term “alkynyl” refers to a straight or branched chain hydrocarbon radical containing from 2 to 12 carbon atoms and at least one carbon to carbon triple bond. Exemplary such groups include ethynyl. “Substituted alkynyl” refers to an alkynyl group substituted with one or more substituents, preferably 1 to 4 substituents, at any available point of attachment. Exemplary substituents include, but are not limited to, alkyl or substituted alkyl, as well as those groups recited above as exemplary alkyl substituents.

The term “cycloalkyl” refers to a fully saturated cyclic hydrocarbon group containing from 1 to 4 rings and 3 to 8 carbons per ring. Exemplary such groups include cyclopropyl, cyclobutyl, cyclopentyl, cyclohexyl, cycloheptyl, etc. “Substituted cycloalkyl” refers to a cycloalkyl group substituted with one or more substituents, preferably 1 to 4 substituents, at any available point of attachment. Exemplary substituents include, but are not limited to, nitro, cyano, alkyl or substituted alkyl, as well as those groups recited above as exemplary alkyl substituents. Exemplary substituents also include spiro-attached or fused cylic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substitutents can themselves be optionally substituted.

The term “cycloalkenyl” refers to a partially unsaturated cyclic hydrocarbon group containing 1 to 4 rings and 3 to 8 carbons per ring. Exemplary such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include but are not limited to nitro, cyano, alkyl or substituted alkyl, as well as those groups recited above as exemplary alkyl substituents. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “aryl” refers to cyclic, aromatic hydrocarbon groups that have 1 to 5 aromatic rings, especially monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). “Substituted aryl” refers to an aryl group substituted by one or more substituents, preferably 1 to 3 substituents, at any point of attachment. Exemplary substituents include, but are not limited to, nitro, cycloalkyl or substituted cycloalkyl, cycloalkenyl or substituted cycloalkenyl, cyano, alkyl or substituted alkyl, as well as those groups recited above as exemplary alkyl substituents. Exemplary substituents also include fused cyclic groups, especially fused cycloalkyl, fused cycloalkenyl, fused heterocycle, or fused aryl, where the aforementioned cycloalkyl, cycloalkenyl, heterocycle and aryl substituents can themselves be optionally substituted.

The terms “heterocycle” and “heterocyclic” refer to fully saturated, or partially or fully unsaturated, including aromatic (i.e., “heteroaryl”) cyclic groups (for example, 4 to 7 membered monocyclic, 7 to 11 membered bicyclic, or 10 to 16 membered tricyclic ring systems) which have at least one heteroatom in at least one carbon atom-containing ring. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, isoindolyl, benzothiazolyl, benzoxazolyl, benzoxadiazolyl, benzo[d][1,3]dioxolyl, benzothienyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl and the like. Exemplary tricyclic heterocyclic groups include carbazolyl, benzidolyl, phenanthrolinyl, acridinyl, phenanthridinyl, xanthenyl and the like.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include, but are not limited to, cycloalkyl or substituted cycloalkyl, cycloalkenyl or substituted cycloalkenyl, nitro, oxo (i.e., ═O), cyano, alkyl or substituted alkyl, as well as those groups recited above as exemplary alkyl substituents. Exemplary substituents also include spiro-attached or fused cylic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “quaternary nitrogen” refers to a tetravalent positively charged nitrogen atom including, for example, the positively charged nitrogen in a tetraalkylammonium group (e.g., tetramethylammonium, N-methylpyridinium), the positively charged nitrogen in protonated ammonium species (e.g., trimethyl-hydroammonium, N-hydropyridinium), the positively charged nitrogen in amine N-oxides (e.g., N-methyl-morpholine-N-oxide, pyridine-N-oxide), and the positively charged nitrogen in an N-amino-ammonium group (e.g., N-aminopyridinium).

The terms “halogen” or “halo” refer to chlorine, bromine, fluorine or iodine.

The term “carbocyclic” refers to aromatic or non-aromatic 3 to 7 membered monocyclic and 7 to 11 membered bicyclic groups, in which all atoms of the ring or rings are carbon atoms. “Substituted carbocyclic” refers to a carbocyclic group substituted with one or more substituents, preferably I to 4 substituents, at any available point of attachment. Exemplary substituents include, but are not limited to, nitro, cyano, ORₐ, wherein Rₐ is as defined hereinabove, as well as those groups recited above as exemplary cycloalkyl substituents.

When a functional group is termed “protected”, this means that the group is in modified form to mitigate, especially preclude, undesired side reactions at the protected site. Suitable protecting groups for the methods and compounds described herein include, without limitation, those described in standard textbooks, such as Greene, T. W. et al., Protective Groups in Organic Synthesis, Wiley, N.Y. (1999).

Unless otherwise indicated, any heteroatom with unsatisfied valences is assumed to have hydrogen atoms sufficient to satisfy the valences.

The compounds of formula I form salts which are also within the scope of this invention. Reference to a compound of the formula I herein is understood to include reference to salts thereof, unless otherwise indicated. The term “salt(s)”, as employed herein, denotes acidic and/or basic salts formed with inorganic and/or organic acids and bases. In addition, when a compound of formula I contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of the compounds of the formula I may be formed, for example, by reacting a compound I with an amount of acid or base, such as an equivalent amount, in a medium such as one in which the salt precipitates or in an aqueous medium followed by lyophilization.

The compounds of formula I which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of formula I which contain an acidic moiety, such but not limited to a carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl) ethylenediamine), N-methyl-D-glucamines, N-methyl-D-glycamides, t-butyl amines, and salts with amino acids such as arginine, lysine and the like. Basic nitrogen-containing groups may be quaternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

Prodrugs and solvates of the compounds of the invention are also contemplated herein. The term “prodrug” as employed herein denotes a compound that, upon administration to a subject, undergoes chemical conversion by metabolic or chemical processes to yield a compound of the formula I, or a salt and/or solvate thereof. Solvates of the compounds of formula I include, for example, hydrates.

Compounds of the formula I, and salts thereof, may exist in their tautomeric form (for example, as an amide or imino ether). All such tautomeric forms are contemplated herein as part of the present invention.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the IUPAC 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

Compounds of the formula I are, subsequent to their preparation, preferably isolated and purified to obtain a composition containing an amount by weight equal to or greater than 99% formula I compound (“substantially pure” compound I), which is then used or formulated as described herein. Such “substantially pure” compounds of the formula I are also contemplated herein as part of the present invention.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

Throughout the specifications, groups and substituents thereof may be chosen to provide stable moieties and compounds.

Biological Assay

The pharmacological properties of the compounds of this invention may be confirmed by a number of pharmacological assays. The exemplified pharmacological assays which follow have been carried out with the compounds according to the invention and/or their pharmaceutically acceptable salts.

A methylation based filter assay was devised to test compounds that specifically inhibited CARM 1 dependent methylation. Human full length CARM1 purified from baculovirus infected insect cells was used as the source for enzyme. Histone H3 (Roche Applied Science) was used as the preferred substrate for the assay since the methylation of several amino acids of Histone H3 by CARM 1 has been well documented. Tritiated S-Adenosyl-L-Methionine (SAM) (Amersham Pharmacia Biotech) was used as a cofactor since CARM1 exhibits an absolute requirement for SAM for its catalytic activity. Methylation reactions were performed for 75-90 minutes at room temperature using enzyme(CARM-1), substrate (Histone H3) and cofactor (SAM) in the presence of methylation buffer (20 mM Tris.HCl pH 8.0, 200 mM NaCl, 0.4 mM EDTA) and in the presence or absence of compound. Reactions were terminated using TCA and precipitated with BSA overnight. They were harvested the next morning by passing the reactions through a filter and the filters washed. The signal on the filters was read in a Top Count after addition of MicroScint-20.

Protocol for Methylation Based Filter Assay

I. Reaction Mixtures and Solutions:

10×Methyl transferase Buffer (MTB):
20 mM Tris HCl, pH 8.0
200 mM NaCl
0.4 mM EDTA

3×Test Compound Solution (15 μl per well):
Test compounds are diluted to 3 fold of final concentration with 1×MTB. For example, if the final concentration of test compound is designed as 30 μM, then 90 μM compound solution should be made at this step.

Diluted DMSO Solution (15 μl per well):
100% DMSO was diluted with 1×MTB to the same concentration as the compound solution. For example, if the 3×concentrate test compound solution contains 3% DMSO then 3% DMSO should be used for control and blank wells.

CARM1 Enzyme Mixture (15 μl per well):
Pre-determined amount of CARM 1 Enzyme
0.03 μl 1M DTT
0.03 μl 100 mg/ml BSA
1.5 μl 10×MTB
13.39 μl H₂O

_Histone H3/[³H] SAM Mixture (15 μl per well):
0.8 μl 1 mg/ml Histone H3
0.2 μl 1 mCi/ml [3H] SAM (65-80 Ci/mmol)
1.5 μl 10×MTB
12.5 μl H₂O

Stop Solution (45 μl per well):
20% TCA
100 mM Sodium pyrophosphate
   II. Reaction Steps:

A plate layout, which contains blank (no enzyme), positive control (no compound) and test compound wells was created

The following reaction mixtures and solutions were added to wells taking care to avoid cross contamination:
15 μl 1×MTB to each of blank wells
15 μl diluted DMSO solution to each of blank and positive control wells
15 μl 3×test compound solution to each of test compound wells
15 μl CARM1 Mixture to each of compound and positive control wells
15 μl Histone H3/[3H] SAM mixture to all wells

Plates were incubated for 60 minutes at room temperature (22-24° C.)

45 μl of stop solution was added to all wells

Plates were left overnight at 4° C.

The next day reaction mixes were harvested to filter plate with a Unifilter harvester

The filter plates were washed twice with 10% TCA and 4 times with H₂O

Air dry filter plate

30 μl Microscint-20 was added to each well and plates were covered with Top-seal

Plates were read in the Top Count and the data was analyzed to generate the IC₅₀ values.

III. The Final Concentration of Components (in 60 μl total reaction volume):

CARM1 Enzyme=as pre-determined

Histone H3=0.8 μg (1.16 μM)

[3H] SAM=0.2 μCi (56 nM)

DMSO=1%

Protocol for 3H Thymidine Incorporation Assay for IC₅₀ Evaluation

Inhibition of tumor cell proliferation upon treatment with compounds was monitored using the 3H thymidine incorporation assay. Cells of appropriate density were plated in 96-well plates and compounds added on the same day. Compound treatment was continued for either 3 or 6 days and dose response curves were determined as described below.

Materials and Reagents RPMI Media
Fetal Bovine Serum
Trypsin-EDTA
DMSO
96 Deep-well plate, 24/box
[6-3H]Thymidine, 5 mCi
Unifilter-96, GF-B
Microscient PS
TopSeal-A:96-Well Microplates
Assay Media:
   RPMImedia+2.5% FBS
Compound dilution:
   A 96 deep-well plate was used for compound dilution. Compounds were diluted 1:3 serially. A 10-point dilution was performed starting at a concentration of 40 μM(2×). Assay media in columns 2-10 contained 0.4% DMSO(2×).

An example is shown below:

| | 1 | 2 | 3 | 4 | 5 | 6 | 7 | 8 | 9 | 10 |
|---|---|---|---|---|---|---|---|---|---|---|
| Conc. μM(2X) | 40 | 13.333 | 4.444 | 1.482 | 0.494 | 0.165 | 0.055 | 0.018 | 0.06 | 0.02 |
| Assay media (ml) | 0.9 | 0.6 | 0.6 | 0.6 | 0.6 | 0.6 | 0.6 | 0.6 | 0.6 | 0.6 |
| 10 μM Compd | 3.6 | | | | | | | | | |
| (μl) | | | | | | | | | | |

Mix well and sequential dilution:

| | | | | | | | | | |
|---|---|---|---|---|---|---|---|---|---|
| Transfer (ml) | 0.3 | 0.3 | 0.3 | 0.3 | 0.3 | 0.3 | 0.3 | 0.3 | 0.3 |

Preparation of cell suspension solution:

2×103-4×104 cells/ml (depending on cell type) of cell suspension was prepared in the assay media. Following is an example:

| | |
|---|---|
| Day 3 | Day 6 |

| Cell line | Cells/well | Cells/ml | Cells/well | Cells/ml |
|---|---|---|---|---|
| A549 | 500 | 1.0 × 10⁴ | 100 | 2 × 10³ |
| | 600 | 1.2 × 10⁴ | 200 | 4 × 10³ |
| | 1000 | 2 × 10⁴ | 500 | 1 × 10⁴ |
| | 2000 | 4 × 10⁴ | 1000 | 2 × 10⁴ |

Plate Cells:
4 compounds were tested per plate.

50 μl of 2×compounds and 50 μl of cell suspension were added per well of a 96-well plate.

| | 1 | 2 | 3 | 4 | 5 | 6 | 7 | 8 | 9 | 10 | 11 | 12 |
|---|---|---|---|---|---|---|---|---|---|---|---|---|
| Conc. | 20 | 6.667 | 2.222 | 0.74 | 0.245 | 0.082 | 0.027 | 0.009 | 0.003 | 0.001 | 0 | |
| μM | | | | | | | | | | | | |

| | |
|---|---|
| A | Compound 1 |
| B | |
| C | Compound 2 |
| D | |
| E | Compound 3 |
| F | |
| G | Compound 4 |
| H | |

Note:
Column 11 was the control. 50 μl of assay media containing 0.4% DMSO and 50 μl of cell suspension were added per well.

On day 3 and day 6:
Addition of [³H] thymidine media:
   Dilution of [³H] thymidine solution: 44 μl of thymidine stock liquid was mixed with 956 μl of assay media for a total of 1 ml. 10 μl of diluted thymidine solution was added into each well (final concentration of thymidine is 4 μCi/well).
Harvest cells:
   Media from the plates was discarded and 75 μl of 0.25% trypsin-EDTA was added to each well. Plates were incubated at 37° C. for 15 min. Cells were then harvested with Unifilter-96 GF-B plate by using the harvester.
Air dry plate:
   Plates were dried overnight.

Data analysis:
The bottom of the plate was sealed with sealing paper.
50 μl of Microscint™ PS was added to each well and the top of the plate was sealed with topseal.
The plate was read on a Topcon instrument and the data was analyzed to generate the IC₅₀ value.

The same protocol was used for the other cancer cell lines.

The compounds of the present invention have been tested in the following cell lines MDA-231 (breast); MDA-453 (breast); MDA-468 (breast); HS-578T (breast); DU-4475 (breast); BT-549 (breast); MCF-7 (breast;) K562 (leukemia); MolT4 (leukemia;) CCRF-CEM (leukemia); OCZ-CY19 (lymphoma); SK-Mel5 (melanoma); SK-Mel28 (melanoma); A549 (lung;) LX1 (lung); H23 (lung); H226 (lung); H522 (lung); H661 (lung); A375 (lung); and MSTO-211H (lung). Additionally, compounds of the present invention may be tested in the following cell lines: SW480 (colon); HCT116 (colon); PC3 (prostate); and LnCaP (prostate).

Representative compounds of the present invention have activity in one or more of the above described assay.

The invention further provides pharmaceutical compositions comprising compounds having formula I together with a pharmaceutically acceptable carrier.

More specifically, the compounds of Formulas I are useful in the treatment of a variety of cancers, including, but not limited to, the following:

a) carcinoma, including that of the bladder, breast, colon, kidney, liver, lung, including small cell lung cancer, esophagus, gall bladder, ovary, pancreas, stomach, cervix, thyroid, prostate, and skin, including squamous cell carcinoma;

b) hematopoietic tumors of lymphoid lineage, including leukemia, acute lymphocytic leukemia, acute lymphoblastic leukemia, B-cell lymphoma, T-cell lymphoma, Hodgkin's lymphoma, non-Hodgkins lymphoma, hairy cell lymphoma and Burkett's lymphoma;

c) hematopoietic tumors of myeloid lineage, including acute and chronic myelogenous leukemias, myelodysplastic syndrome and promyelocytic leukemia;

d) tumors of mesenchymal origin, including fibrosarcoma and rhabdomyosarcoma;

e) tumors of the central and peripheral nervous system, including astrocytoma, neuroblastoma, glioma and schwannomas; and

f) other tumors, including melanoma, seminoma, teratocarcinoma, osteosarcoma, xenoderoma pigmentosum, keratoctanthoma, thyroid follicular cancer and Kaposi's sarcoma.

The compounds of Formula I are useful in the treatment of breast cancer, leukemia, melanoma, lung cancer, colon cancer and prostate cancer.

In another embodiment of the invention, a method is provided for treating a proliferative disease via modulation of CARM-1 (PRMT-4) by administering to a patient in need of such treatment an effective amount of a compound of formula I, as defined above, in combination (simultaneously or sequentially) with at least one other anti-cancer agent. In a another embodiment, the proliferative disease is cancer.

The compounds of this invention may also be useful in combination with known anti-cancer and cytotoxic agents and treatments, including radiation. If formulated as a fixed dose, such combination products employ the compounds of this invention within the dosage range described below and the other pharmaceutically active agent may be useful within its approved dosage range or within a range determined to be useful. Compounds of formula I may be used sequentially with known anticancer or cytotoxic agents and treatment, including radiation when a combination formulation is inappropriate.

In medical oncology, the other component(s) of such conjoint treatment in addition to the antiproliferative, antiangiogenic, and/or vascular permeability reducing treatment defined herein before may be: surgery, radiotherapy, or chemotherapy. Such chemotherapy may cover three main categories of therapeutic agent:

(i) antiangiogenic agents that work by different mechanisms from those defined hereinbefore (for example, linomide, inhibitors of integrin αvβ3 function, angiostatin, and razoxane);

(ii) cytostatic agents such as antiestrogens (for example tamoxifen, toremifene, raloxifene, droloxifene, and iodoxifene), progestogens (for example megestrol acetate), aromatase inhibitors (for example anastrozole, letrozole, borazole, and exemestane), antihormones, antiprogestogens, antiandrogens (for example flutamide, nilutamide, bicalutamide, and cyproterone acetate), LHRH agonists and antagonists (for example gosereline acetate and leuprolide), inhibitors of testosterone 5α-dihydroreductase (for example finasteride), farnesyltransferase inhibitors, anti-invasion agents (for example metalloproteinase inhibitors like marimastat and inhibitors of urokinase plasminogen activator receptor function) and inhibitors of growth factor function, (such growth factors include for example EGF, FGF, platelet derived growth factor and hepatocyte growth factor such as growth factor antibodies, growth factor receptor antibodies such as Avastin® (bevacizumab) and Erbitux® (cetuximab); tyrosine kinase inhibitors (protein tyrosine kinase inhibitors), and serine/threonine kinase inhibitors); and

(iii) antiproliferative/antineoplastic drugs and combinations thereof, as used in medical oncology, such as antimetabolites (for example antifolates like methotrexate, fluoropyrimidines like 5-fluorouracil, purine and adenosine analogues, cytosine arabinoside, and pyrimidine analogues); intercalating antitumor antibiotics (for example anthracyclines like doxorubicin, daunomycin, epirubicin, idarubicin, mitomycin-C, dactinomycin, and mithramycin, cytarabine (ara-C; Cytosar-U®)); platinum derivatives (for example cisplatin and carboplatin); alkylating agents (for example nitrogen mustard, melphalan, chlorambucil, busulphan, cyclophosphamide, ifosfamide nitrosoureas, thiotepa; antimitotic agents (for example vinca alkaloids like vincristine and taxoids like Taxol® (paclitaxel), Taxotere® (docetaxel) and newer microbtubule agents such as epothilone analogs, and epothilones A-F or their analogs or derivatives;, discodermolide analogs, and eleutherobin analogs); topoisomerase inhibitors (for example epipodophyllotoxins like etoposide, teniposide, amsacrine, and topotecan); cell cycle inhibitors (for example flavopyridols); biological response modifiers, and proteasome inhibitors such as Velcade® (bortezomib).

Additionally, the compounds of the present invention may be employed alone or in combination with other suitable therapeutic agents useful in the treatment of cancer such as antiinflammatories and immunosuppressants.

Exemplary classes of anti-cancer agents and cytotoxic agents include, but are not limited to: alkylating agents, such as nitrogen mustards, alkyl sulfonates, nitrosoureas, ethylenimines, and triazenes; 6-thioguanine (Tabloid®), mitoxantrone (Novantrone®) and etoposide (VePesid®), amsacrine (AMSA), and all-trans retinoic acid (ATRA), bleomycins, mitomycin, dactinomycin, and plicamycin; enzymes, such as L-asparaginase; farnesyl-protein transferase inhibitors; hormonal agents, such as glucocorticoids, estrogens/antiestrogens, androgens/antiandrogens, progestins, and luteinizing hormone-releasing hormone anatagonists, octreotide acetate; microtubule-disruptor agents, such as ecteinascidins or their analogs and derivatives; plant-derived products, such as vinca alkaloids, epipodophyllotoxins, taxanes; and topoisomerase inhibitors; prenyl-protein transferase inhibitors; and miscellaneous agents such as, hydroxyurea, procarbazine, mitotane, hexamethylmelamine,; and other agents used as anti-cancer and cytotoxic agents such as biological response modifiers, growth factors; immune modulators, and monoclonal antibodies. The compounds of the invention may also be used in conjunction with radiation therapy.

Representative examples of these classes of anti-cancer and cytotoxic agents include, but are not limited to, mechlorethamine hydrochloride, busulfan, carmustin, lomustine, semustine, streptozocin, dacarbazine, thioguanine, mercaptopurine, fludarabine, pentastatin, cladribin, doxorubicin hydrochloride, daunorubicin, bleomycin sulfate, mitomycin C, actinomycin D, safracins, saframycins, quinocarcins, discodermolides, vinblastine, vinorelbine tartrate, estramustine, estramustine phosphate sodium, buserelin, pteridines, diyneses, levamisole, aflacon, aldesleukin, filgrastim, sargramostim, rituximab, BCG, tretinoin, irinotecan hydrochloride, betamethosone, gemcitabine hydrochloride, altretamine, and topoteca and any analogs or derivatives thereof.

The term “anticancer” agent includes any known agent that is useful for the treatment of cancer including the following: 17α-ethinylestradiol, diethylstilbestrol, testosterone, prednisone, fluoxymesterone, dromostanolone propionate, testolactone, megestrolacetate, methylprednisolone, methyl-testosterone, prednisolone, triamcinolone, chlorotrianisene, hydroxyprogesterone, aminoglutethimide, medroxyprogesteroneacetate, leuprolide, flutamide, toremifene, Zoladex; matrix metalloproteinase inhibitors; VEGF inhibitors, such as anti-VEGF antibodies (Avastin) and small molecules such as ZD6474, AZD-2171, SU6668; Vatalanib, BAY43-9006, SU11248, CP-547632, and CEP-7055; Her 1 and Her 2 inhibitors including anti-Her2 antibodies (Herceptin); EGFR inhibitors including gefitinib, erlotinib, ABX-EGF, EMD72000, 11F8, EKB-569, and cetuximab; Imclone antibody C225 immunospecific for the EGFR; Eg5 inhibitors, such as SB-715992, SB-743921, and MKI-833; pan Her inhibitors, such as canertinib, EKB-569, C₁-1033, AEE-788, XL-647, mAb 2C4, and GW-572016; Src kinase inhibitors such as BMS-354825, AZD-0530, SKI-606, and AP-23464; Bcr-Abl inhibitors such as imatinib and AMN107; Casodex® (bicalutamide, Astra Zeneca), MEK-1 kinase inhibitors, MAPK kinase inhibitors, PI3 kinase inhibitors; Met inhibitors, Aurora kinase inhibitors, PDGF inhibitors; anti-angiogenic and antivascular agents which, by interrupting blood flow to solid tumors, render cancer cells quiescent by depriving them of nutrition; castration, which renders androgen dependent carcinomas non-proliferative; IGF1R inhibitors such as those disclosed in US2004/44203A1, inhibitors of non-receptor and receptor tyrosine kinases (such as Iressa and OSI-774); c-Kit inhibitors; inhibitors of integrin signaling; tubulin acting agents such as vinorelbine, vinflunine, docetaxel, 7-O-methylthiomethylpaclitaxel, 4-desacetyl-4-methylcarbonatepaclitaxel, 3′-tert-butyl-3′-N-tert-butyloxycarbonyl-4-deacetyl-3′-dephenyl-3′-N-debenzoyl-4-O-methoxycarbonyl-paclitaxel, C-4 methyl carbonate paclitaxel, epothilone A, epothilone B, epothilone C, epothilone D, desoxyepothilone A, desoxyepothilone B, ixabepilone, [1S-[1R*,3R*(E),7R*,10S*,11R*,12R*,16S*]]-3-[2-[2-(aminomethyl)-4-thiazolyl]-1-methylethenyl]-7,11-dihydroxy-8,8,10,12,16-pentamethyl-4-17-dioxabicyclo[14.1.0]-heptadecane-5,9-dione, and derivatives thereof; CDK inhibitors, antiproliferative cell cycle inhibitors, epidophyllotoxin, etoposide, VM-26; antineoplastic enzymes, e.g., topoisomerase I inhibitors, camptothecin, topotecan, SN-38; procarbazine; mitoxantrone; platinum coordination complexes such as cisplatin, carboplatin and oxaliplatin; biological response modifiers; growth inhibitors; antihormonal therapeutic agents; leucovorin; tegafur; antimetabolites such as purine antagonists (e.g. 6-thioguanine and 6-mercaptopurine; glutamine antagonists, e.g. DON (AT-125; d-oxo-norleucine); ribonucleotide reductase inhibitors; mTOR inhibitors; and haematopoietic growth factors.

Additional cytotoxic agents include, cyclophosphamide, mitoxanthrone, melphalan, hexamethyl melamine, thiotepa, cytarabin, idatrexate, trimetrexate, dacarbazine, L-asparaginase, bicalutamide, pyridobenzoindole derivatives, interferons, and interleukins.

In cases where it is desirable to render aberrantly proliferative cells quiescent in conjunction with or prior to treatment with the chemotherapeutic methods of the invention, hormones and steroids (including synthetic analogs): 17a-Ethinylestradiol, Diethylstilbestrol, Testosterone, Prednisone, Fluoxymesterone, Dromostanolone propionate, Testolactone, Megestrolacetate, Methylprednisolone, Methyl-testosterone, Prednisolone, Triamcinolone, hlorotrianisene, Hydroxyprogesterone, Aminoglutethimide, Estramustine, Medroxyprogesteroneacetate, Leuprolide, Flutamide, Toremifene, Zoladex can also be administered to the patient.

As mentioned, certain anti-proliferative agents are anti-angiogenic and antivascular agents and, by interrupting blood flow to solid tumors, render cancer cells quiescent by depriving them of nutrition. Castration, which also renders androgen dependent carcinomas non-proliferative, may also be utilized. Starvation by means other than surgical disruption of blood flow is another example of a cytostatic agent. A particular class of antivascular cytostatic agents is the combretastatins.

Compounds of Formulas I as modulators of apoptosis, may be useful in the treatment of cancer (including but not limited to those types mentioned herein above), viral infections (including but not limited to herpevirus, poxvirus, Epstein-Barr virus, Sindbis virus and adenovirus), prevention of AIDS development in HIV-infected individuals, autoimmune diseases (including but not limited to systemic lupus, erythematosus, autoimmune mediated glomerulonephritis, rheumatoid arthritis, psoriasis, inflammatory bowel disease, and autoimmune diabetes mellitus), neurodegenerative disorders (including but not limited to Alzheimer's disease, AIDS-related dementia, Parkinson's disease, amyotrophic lateral sclerosis, retinitis pigmentosa, spinal muscular atrophy and cerebellar degeneration), myelodysplastic syndromes, aplastic anemia, ischemic injury associated with myocardial infarctions, stroke and reperfusion injury, arrhythmia, atherosclerosis, toxin-induced or alcohol related liver diseases, hematological diseases (including but not limited to chronic anemia and aplastic anemia), degenerative diseases of the musculoskeletal system (including but not limited to osteoporosis and arthritis) aspirin-sensitive rhinosinusitis, cystic fibrosis, multiple sclerosis, kidney diseases and cancer pain.

Compounds of Formulas I may modulate the level of cellular RNA and DNA synthesis. These agents would therefore be useful in the treatment of viral infections (including but not limited to HIV, human papilloma virus, herpesvirus, poxvirus, Epstein-Barr virus, Sindbis virus and adenovirus).

Compounds of Formulas I may be useful in the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse.

Compounds of Formulas I may also be useful in inhibiting tumor angiogenesis and metastasis.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropyl-methylcellulose or hydroxypropyl-cellulose, or a time delay material such as ethyl cellulose, cellulose acetate buryrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS.™. model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of Formulas I may also be administered in the form of a suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of Formula I are employed. (For purposes of this application, topical application shall include mouth washes and gargles.)

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

When a compound according to this invention is administered into a human subject, the daily dosage will normally be determined by the prescribing physician with the dosage generally varying according to the age, weight, sex and response of the individual patient, as well as the severity of the patient's symptoms.

If formulated as a fixed dose, such combination products employ the compounds of this invention within the dosage range described above and the other pharmaceutically active agent or treatment within its approved dosage range. Compounds of Formula I may also be administered sequentially with known anticancer or cytotoxic agents when a combination formulation is inappropriate. The invention is not limited in the sequence of administration; compounds of Formula I may be administered either prior to or after administration of the known anticancer or cytotoxic agent(s).

In another embodiment, the present invention is directed to compounds of formula I, Ia, Ib or Id, wherein
Ring Q is 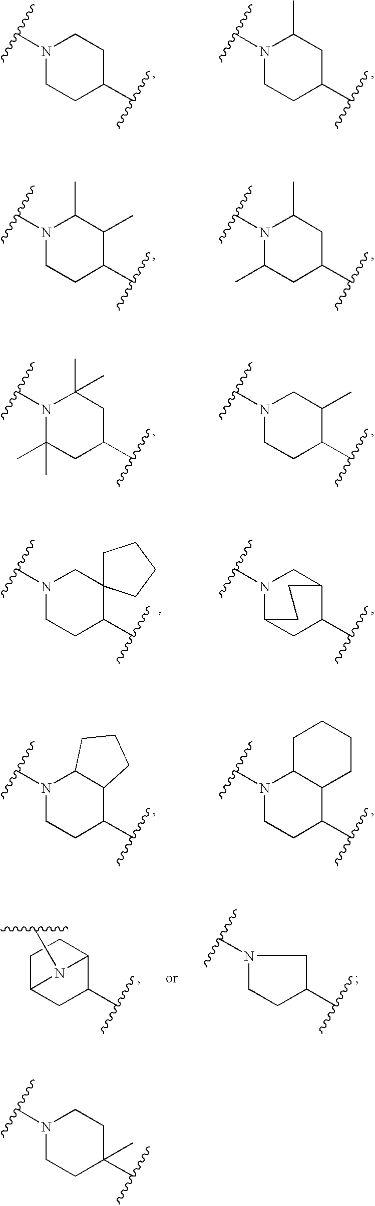
X is C (i.e., carbon) or N (i.e., nitrogen);
Y is NH, N-Me, or CH;
A, B and D are each independently N or C, in which C may be optionally substituted with H, Me, Et, halogen, CN, NO₂, OMe, OEt, SMe, SO₂Me, CF₃, or OCF₃;
R₁ is aryl, substituted aryl, heterocycle, or substituted heterocycle;
R₂ is H, Me, Et, halogen, CN, NO₂, OMe, OEt, SMe, SO₂Me, CF₃, or OCF₃, provided that when X is N, R₂ is nil;
R₃ is H or C₁-C₄ alkyl; and
n is 1, 2, or 3.

Another embodiment of the compounds of the present invention includes compounds of the formula I and/or Id, or a stereoisomer, a tautomer, a pharmaceutically acceptable salt or solvate thereof, wherein one or more of the following substituents are as defined below:
Ring Q is 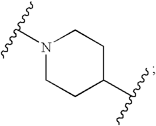
X is C (i.e., carbon) or N (i.e., nitrogen);
Y is NH or CH;
A, B and D are each independently C, which may be optionally substituted with H, Me, Et, halogen, CN, NO₂, OMe, OEt, SMe, SO₂Me, CF₃, or OCF₃;
R₁ is aryl, substituted aryl, heteroaryl or substituted heteroaryl;
R₂ is H, Me, Et, halogen, CN, OMe, OEt, SMe, SO₂Me, CF₃, or OCF₃, provided that when X is N, R₂is nil;
R₃ is H or C₁-C₄ alkyl; and
n is 1.

Another embodiment of the compounds of the invention includes compounds of the formula I having the following structure Ia, or a stereoisomer, a tautomer, a pharmaceutically acceptable salt or solvate thereof, wherein one or more, preferably all, of the following substituents are as defined below: 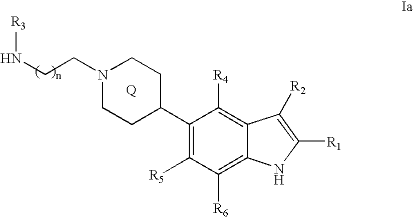
Ring Q is 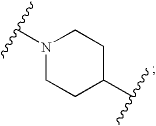
R₁ is aryl, substituted aryl, heteroaryl or substituted heteroaryl;
R₂, R₄, R₅ and R₆ are each independently H, Me, Et, halogen, CN, OMe, OEt, SMe, SO₂Me, CF₃, or OCF₃;
R₃ is H or C₁-C₄ alkyl; and
n is 1.

In another embodiment, the present invention is directed to compounds wherein the compound is of formula Ia, or a stereoisomer, a tautomer, a pharmaceutically acceptable salt or solvate thereof,

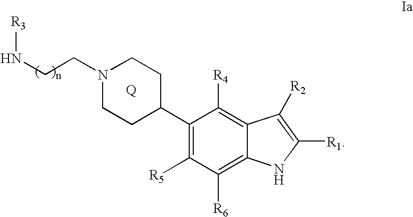

In another embodiment, the present invention is directed to compounds wherein the compound is of formula Ib, or a stereoisomer, a tautomer, a pharmaceutically acceptable salt or solvate thereof,

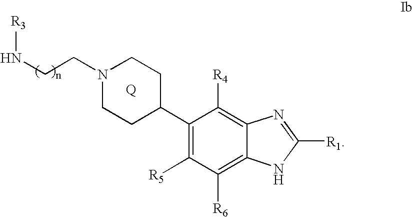

In another embodiment, the present invention is directed to compounds wherein the compound is of formula Id, or a stereoisomer, a tautomer, a pharmaceutically acceptable salt or solvate thereof,

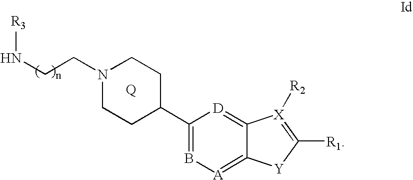

In another embodiment, the compounds include the following substituents as defined below:
Ring Q is 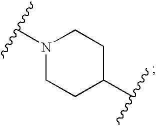
R₁ is aryl, substituted aryl, heteroaryl or substituted heteroaryl;
R₂ is H, Me, halogen, or CN;
R₄ and R₅ are each independently H;
R₃ is Me; and
n is 1.

Another embodiment of the compounds of the invention includes compounds of the formula I having the following structure Ib, or a stereoisomer, a tautomer, a pharmaceutically acceptable salt or solvate thereof, wherein one or more, preferably all, of the following substituents are as defined below: 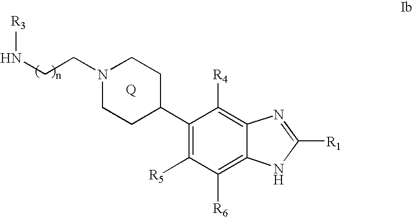
Ring Q is 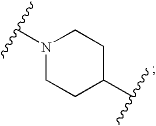
R₁ is aryl, substituted aryl, heteroaryl or substituted heteroaryl;
R₄, R₅ and R₆ are each independently H, Me, Et, halogen, CN, OMe, OEt, SMe, SO₂Me, CF₃, or OCF₃;
R₃ is H or C₁-C₄ alkyl; and
n is 1.

In another embodiment, the compounds include the following substituents as defined below:
Ring Q is 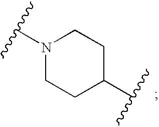
R₁ is aryl, substituted aryl, heteroaryl or substituted heteroaryl;
R₄ and R₅ are each independently H;
R₆ is H or Me;
R₃ is Me; and
n is 1.

In another embodiment, the present invention is directed to compouns of formula I, Ia, Ib, and/or Id, wherein ring Q is

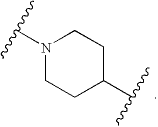

In another embodiment, the present invention is directed to compouns of formula I, Ia, Ib, and/or Id, wherein A, B and D are each independently C, which may be optionally substituted with H. Me, Et, halogen, CN, NO₂, OMe, OEt, SMe, SO₂Me, CF₃, or OCF₃.

In another embodiment, the present invention is directed to compouns of formula I, Ia, Ib, and/or Id, wherein R₁ is aryl or substituted aryl.

In another embodiment, the present invention is directed to compouns of formula I, Ia, Ib, and/or Id, wherein R₁ is heteroaryl or substituted heteroaryl.

In another embodiment, the present invention is directed to compouns of formula I, Ia, Ib, and/or Id, wherein A, B and D are each independently C, which may be optionally substituted with H, Me, Et, halogen, CN, NO₂, OMe, OEt, SMe, SO₂Me, CF₃, or OCF₃.

In another embodiment, the present invention is directed to compouns of formula I, Ia, Ib, and/or Id, wherein R₁ is aryl, substituted aryl, heteroaryl or substituted heteroaryl.

In another embodiment, the present invention is directed to compouns of formula I, Ia, Ib, and/or Id, wherein n is 1.

In another embodiment, the present invention is directed to compouns of formula I, Ia, Ib, and/or Id, wherein R₃ is Me.

In another embodiment, the invention is a pharmaceutical composition compising at least one compound according to Formula I, Ia, Ib, and/or Id, and a pharmaceutically-acceptable carrier or diluent.

In another embodiment, the invention is directed to a pharmaceutical composition comprising at least one compound according to formula I, Ia, Ib, and/or Id, and further comprising at least one other anti cancer agent or cytotoxic agent.

In another embodiment, the invention is directed to a pharmaceutical composition, wherein said anti-cancer or cytotoxic agent is selected from the group consisting of tamoxifen, toremifene, raloxifene, droloxifene, iodoxifene, megestrol acetate, anastrozole, letrozole, borazole, exemestane, flutamide, nilutamide, bicalutamide, cyproterone acetate, gosereline acetate, leuprolide, finasteride, metalloproteinase inhibitors, inhibitors of urokinase plasminogen activator receptor function, growth factor antibodies, growth factor receptor antibodies, bevacizumab, cetuximab, tyrosine kinase inhibitors, serine/threonine kinase inhibitors, methotrexate, 5-fluorouracil, purine and adenosine analogues, cytosine arabinoside, doxorubicin, daunomycin, epirubicin, idarubicin, mitomycin-C, dactinomycin, mithramycin, cisplatin, carboplatin, nitrogen mustard, melphalan, chlorambucil, busulphan, cyclophosphamide, ifosfamide, nitrosoureas, thiotepa, vincristine, vinorelbine, vinblastine, vinflunine, paclitaxel, docetaxel, epothilone analogs, discodermolide analogs, eleutherobin analogs, etoposide, teniposide, amsacrine, topotecan, flavopyridols, proteasome inhibitors including bortezomib and biological response modifiers, androgen receptor antagonists, LH/RH antagonists, taxane analogues, and estrogen receptor antagonists.

In another embodiment, the present invention is directed to a method of inhibiting the activity of protein arginine methyl transferases which comprises administering to a mammalian species in need thereof an effective amount of at least one compound of formula I, Ia, Ib, and/or Id, or a stereoisomer, a tautomer, a pharmaceutically acceptable salt or solvate thereof,

In another embodiment, the pesent invention is directed to a method for treating a condition or disorder comprising administering to a mammalian species in need thereof a therapeutically effective amount of at least one compound of Formula I, Ia, Ib and/or Id, or a stereoisomer, a tautomer, a pharmaceutically acceptable salt or solvate thereof,

wherein said condition or disorder is selected from the group consisting of proliferate diseases, cancers, benign prostate hypertrophia, benign prostatic hyperplasia, adenomas and neoplasies of the prostate, benign or malignant tumor cells containing the androgen receptor, brain cancer, skin cancer, bladder cancer, lymphatic cancer, liver cancer, kidney cancer, pancreatic cancer, prostate cancer, hirsutism, acne, precocious puberty, angiogenic conditions or disorders, hyperpilosity, inflammation, immune modulation, seborrhea, endometriosis, polycystic ovary syndrome, androgenic alopecia, hypogonadism, osteoporosis, suppressing spermatogenesis, male and female sexual dysfunction, libido, cachexia, anorexia, inhibition of muscular atrophy in ambulatory patients, androgen supplementation for age related decreased testosterone levels in men, cancers expressing the estrogen receptor, breast cancer, ovarian cancer, uterine cancer, endometrial cancer, hot flushes, vaginal dryness, menopause, amennoreahea, dysmennoreahea, contraception, pregnancy termination, cancers containing the progesterone receptor, cyclesynchrony, meniginoma, fibroids, labor induction, autoimmune diseases, Alzheimer's disease, psychotic disorders, drug dependence, non-insulin dependent Diabetes Mellitus, dopamine receptor mediated disorders, heart disease, congestive heart failure, disregulation of cholesterol homeostasis, and attenuating the metabolism of a pharmaceutical agent. In another embodiment, the present invention is directed to a compound of formula (I) for use in therapy.

In another embodiment, the present invention is directed to a compound of formula I, Ia, Ib, and/or Id, for the use in the preparation of a medicament for the treatment of cancer.

The invention may be embodied in other specific forms without departing from the spirit or essential attributes thereof. This invention also encompasses all combinations of alternative aspects of the invention noted herein. It is understood that any and all embodiments of the present invention may be taken in conjunction with any other embodiment to describe additional embodiments of the present invention. Furthermore, any elements of an embodiment are meant to be combined with any and all other elements from any of the embodiments to describe additional embodiments.

### METHODS OF PREPARATION

The compounds of the present invention may be prepared by methods such as those illustrated in the following schemes. Solvents, temperatures, pressures, and other reaction conditions may readily be selected by one of ordinary skill in the art. Starting materials are commercially available or readily prepared by one of ordinary skill in the art. These schemes are illustrative and are not meant to limit the possible techniques one skilled in the art may use to manufacture compounds disclosed herein. Different methods may be evident to those skilled in the art. Additionally, the various steps in the synthesis may be performed in an alternate sequence or order to give the desired compound(s). All documents cited herein are incorporated herein by reference in their entirety.

In general, the time taken to complete a reaction procedure will be judged by the person performing the procedure, preferably with the aid of information obtained by monitoring the reaction by methods such as HPLC or TLC. A reaction does not have to go to completion to be useful to this invention. The methods for the preparation of various heterocycles used to this invention can be found in standard organic reference books, for example, “Comprehensive Heterocyclic Chemistry, The Structure, Reactions, Synthesis and Uses, of Heterocyclic Compounds,” Katritzky, A. R., Rees, C. W. Eds Pergamon Press New York, First edition 1984, and “Comprehensive Heterocyclic Chemistry II, A Review of the Literature 1982-1995: The Structure, Reactions, Synthesis and Uses, of Heterocyclic Compounds,” Katritzky, A. R., Rees, C. W. and Scriven, E., F. Eds Pergamon Press New York, 1996.

Unless otherwise specified, the various substituents of the compounds are defined in the same manner as the formula I compound of the invention.

Compounds of general formula I where X is N can be made using a general reaction sequence as depicted in Scheme 1. Suitably halo substituted ortho nitro aniline derivative 2 can be converted into the required boronic acid or stannane derivative 3 using transition metal mediated coupling. Compound 3 can be coupled with Q′-LG (in which LG is a leaving group such as triflate or halogen and ring Q′ has an olefin adjacent to its leaving group LG; and in ring Q′, nitrogen is protected by an amine protecting group PG) to afford compound 4 in the presence of transition metal catalyst. Reduction of nitro and olefin functionality gives ortho-phenelynediamine 5. Coupling of compound 5 with an aldehyde (R₁CHO) or acid (R₁COOH) followed by cyclization gives compound 6 (Note: its imidazole portion can exist in tautomeric forms). Removal of the protective group PG in compound 6, followed by coupling with an appropriately protected amine PG-N(R₃)(CH₂)ₙCH₂CHO or PG-N(R₃)(CH₂)ₙCH₂CH₂-LG (LG is a leaving group such as triflate or halogen), and further followed by final deprotection gives the compound of formula I-1. 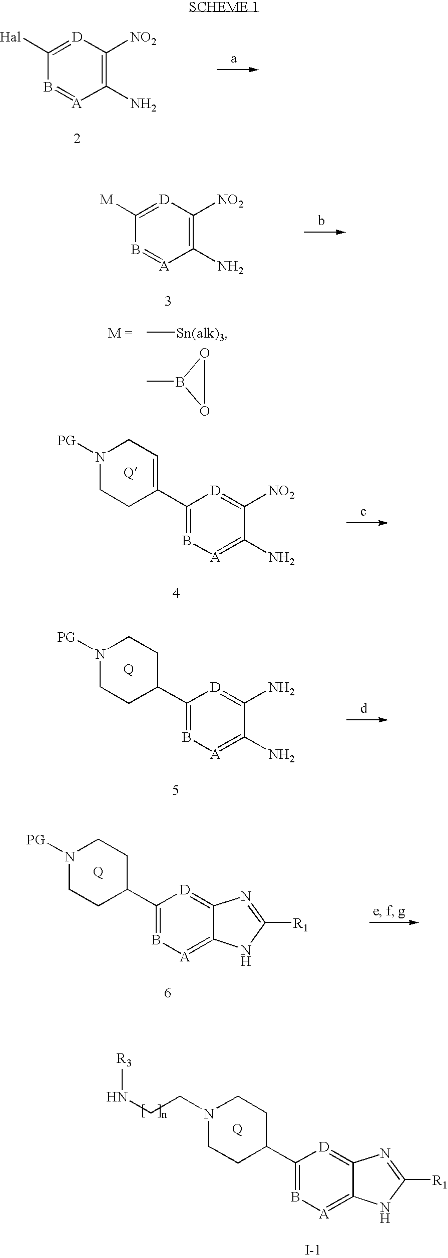
a) Pd(0), (Sn)₂(alk)₆ or (RO)₃B b) Q′-LG, transition metal c) reduction; d) R₁CHO or R₁COOH, coupling reagent; e) PG removal; f) PG-N(R₃)(CH2)ₙCH₂CHO or PG-N(R₃)(CH₂)ₙCH₂CH₂-LG; NaBH(OAc)₃ or NaCHBH₃; g) PG removal

Likewise compounds of formula I with X is C and Y is NH can be synthesized according to Scheme 2. Selective coupling of acetylene derivative with dihalo-aniline derivative 7 gives compound 8, which can be cyclized to give the indole derivative 9 in the presence of a transition metal catalyst such as Pd (0). Compound 9 can be converted to a boronic acid ester or stannane derivative of formula 10 which is coupled with Q′-LG as discussed above, followed by reduction of the olefin affords compound 11. Removal of the protective group PG in compound 11, followed by coupling with an appropriately protected amine PG-N(R₃)(CH₂)ₙCH₂CHO or PG-N(R₃)(CH₂)ₙCH₂CH₂-LG (LG is a leaving group such as triflate or halogen), and further followed by final deprotection gives the compound of formula I-2. 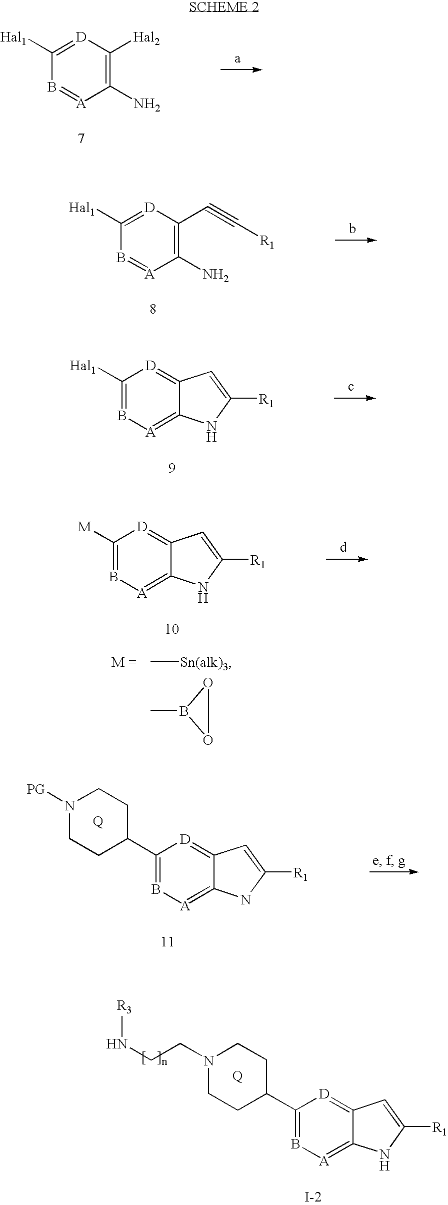
a) Transition metal, R₁C≡CH; b) transition metal; c) transition metal, Sn (alk)₆ or B(OR)₃; d) (i) Q′-LG, transition metal; (ii) H₂, Pd-(C); e) PG deprotection; f) PG-N(R₃)(CH2)ₙCH₂CHO or PG-N(R₃)(CH₂)ₙCH₂CH₂-LG; NaBH(OAc)₃ or NaCHBH₃; g) PG removal

Similarly, compounds of formula I wherein X is N, R₂ is H, and Y is CH can be prepared in accordance with Scheme 3. 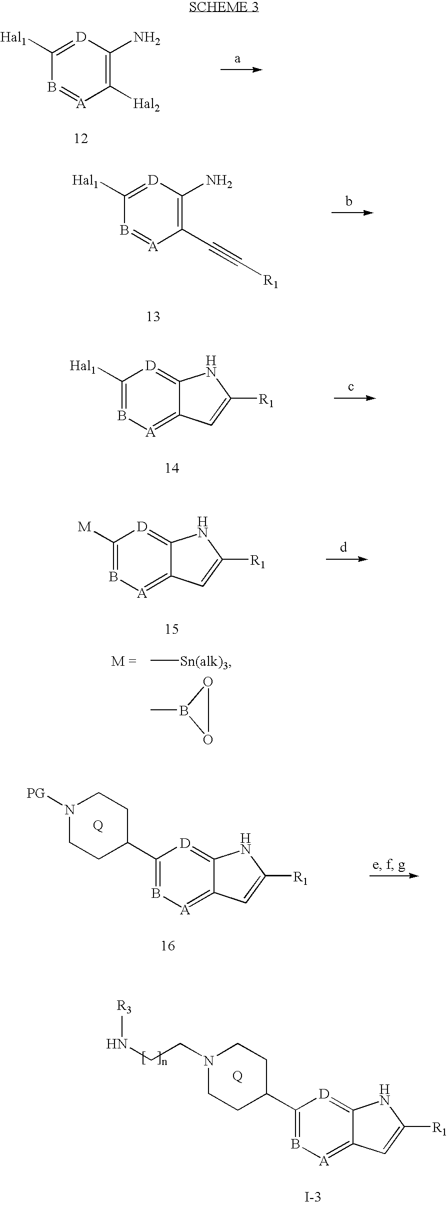
a) Transition metal, R₁C≡CH; b) transition metal; c) transition metal, Sn (alk)₆ or B(OR)₃; d) (i) Q′-LG, transition metal; (ii) H₂, Pd - C; e) PG deprotection; f) PG-N(R₃)(CH2)ₙCH₂CHO or PG-N(R₃)(CH₂)ₙCH₂CH₂-LG; NaBH(OAc)₃ or NaCHBH₃; g) PG removal

Compounds of formula I wherein X is carbon, R₂ is halogen and Y as NH, can be prepared from the intermediate 11 as shown in Scheme 4. Compound 17 can be prepared from compound 11 using a similar method as discussed above. Halogenation of compound 17 using a halogenation reagent such as NBS, NCS, NIS or SelectFluor® followed by deprotection of the PG group gives the compound of formula I-4. 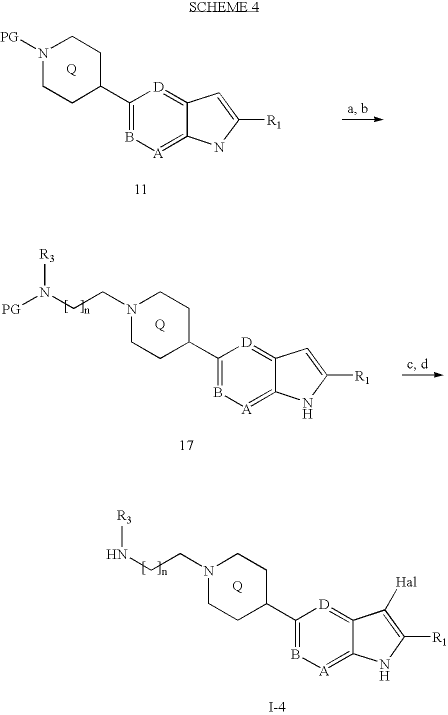
a) PG deprotection; b) PG-N(R₃)(CH2)ₙCH₂CHO or PG-N(R₃)(CH₂)ₙCH₂CH₂-LG; NaBH(OAc)₃ or NaCHBH₃; c) Halogenation (e.g., NBS or NCS or SelectFlor); d) PG deprotection

In an analogous manner, compounds of formula I wherein X=N, R₂ is H, and Y=C-Hal can be synthesized from intermediate 16 as shown in Scheme 5. 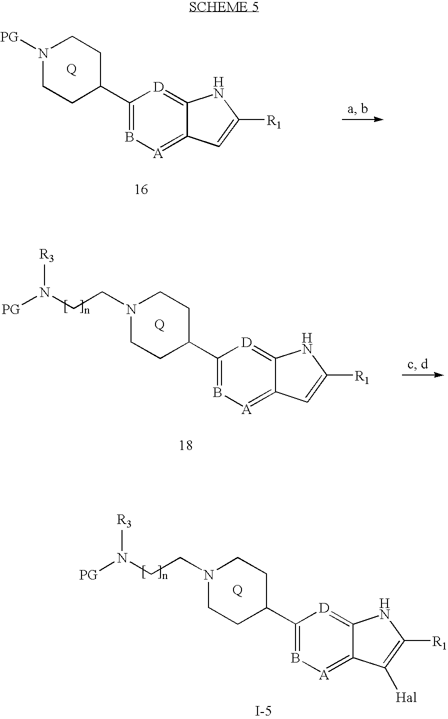
a) PG deprotection; b) PG-N(R₃)(CH2)ₙCH₂CHO or PG-N(R₃)(CH₂)ₙCH₂CH₂-LG; NaBH(OAc)₃ or NaCHBH₃; c) Halogenation (e.g., NBS or NCS or SelectFlor); d) PG deprotection

As shown in Scheme 6, compounds of formula I wherein X is C, R₂ is CN, and Y is NH can be made from the corresponding intermediate 17 described above, through 3-cyanation (using the procedure as reported in Bioorg. Med. Chem. Letters 2003, 1273) followed by deprotection. 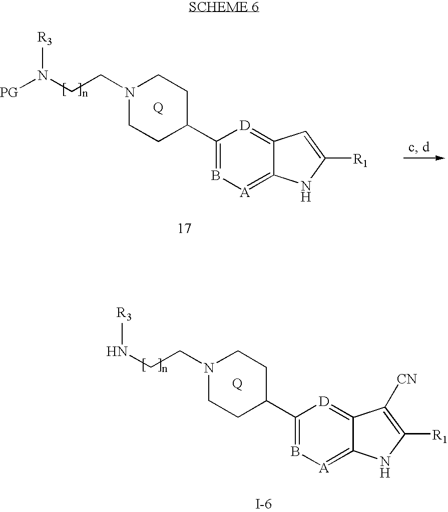
a) ClSO₂NCO; b) PG deprotection

Scheme 7 describes the synthesis of compounds of formula I wherein X is carbon, R₂ is C₁-C₄ alkyl and Y is NH. Aniline derivative 7 could be coupled with acetylene derivative 19 using transition metal catalyst to yield indole derivative 20. The compound of formula I can be obtained from compound 20 through intermediates 21 and 22 using an analogous method as discussed above. 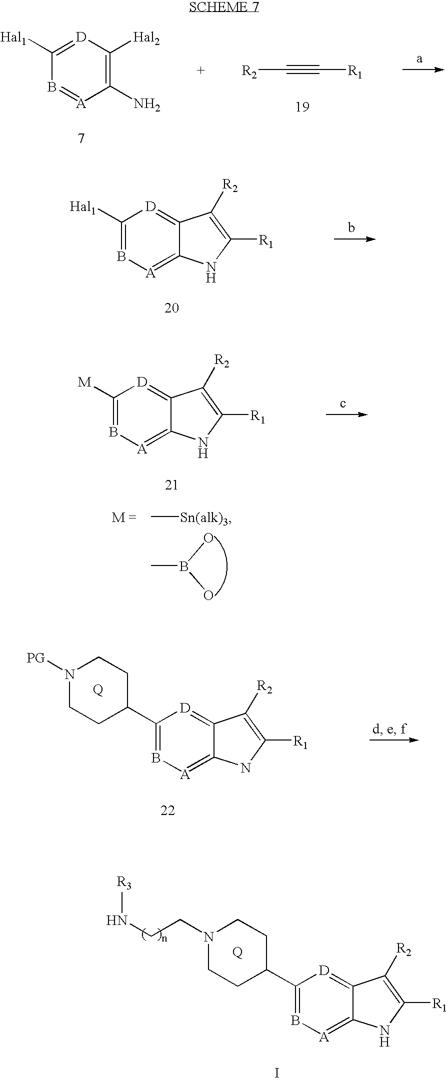
a) Transition metal, R1CCH; b) transition metal, Sn (alk)₆ or B(OR)₃; c) (i) Q′-LG; transition metal, (ii) H₂, Pd-C; d) PG deprotection; e) PG-N(R₃)(CH2)ₙCH₂CHO or PG-N(R₃)(CH₂)ₙCH₂CH₂-LG; NaBH(OAc)₃ or NaCHBH₃; f) PG removal

Alternatively, compounds of formula I wherein X is carbon, R₂ is C₁-C₄ alkyl and Y is NH can be prepared according to Scheme 8. 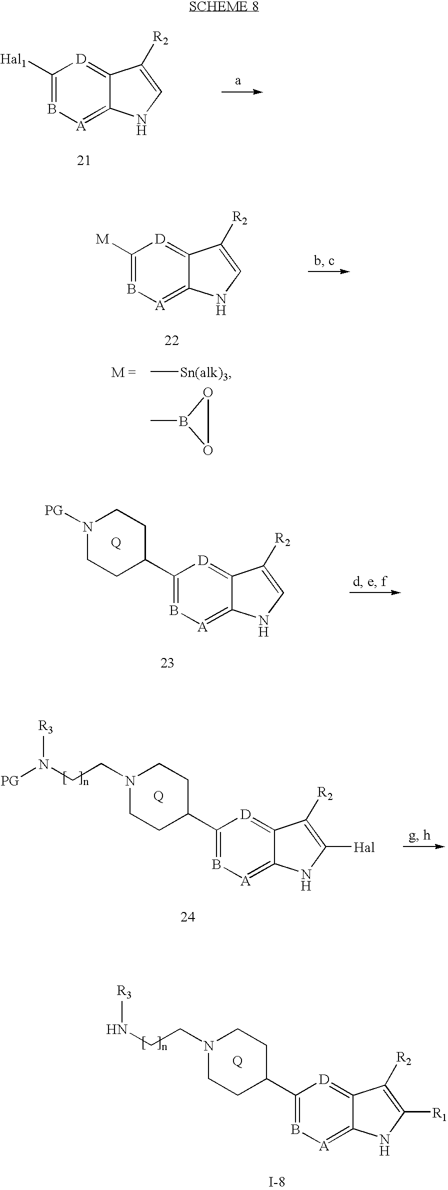
a) transition metal, Sn (alk)₆ or B(OR)₃; b) Q′-LG; transition metal; c) H₂, Pd; d) PG deprotection; e) PG-N(R₃)(CH2)ₙCH₂CHO or PG-N(R₃)(CH₂).CH₂CH₂-LG; NaBH(OAc)₃ or NaCHBH₃; f) Halogenation (e.g., Br₂); g) R₁-M (M=Sn, B), transition metal; h) PG removal.

Compounds of formula I wherein X is carbon, R₂ is C₁-C₄ alkyl, R1 is aryl or heteroaryl, Y is NH and the bond between X and carbon is single bond can be prepared according to Scheme 9. 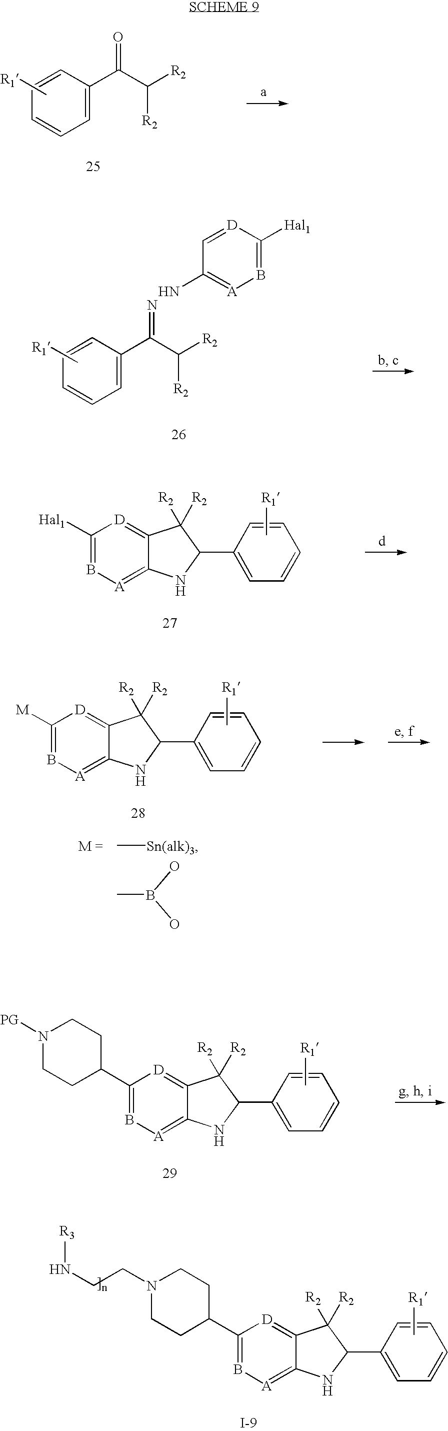
a) R1-NHNH2; b) AcOH, heat; c) NaBH4, methanol; d) transition metal, Sn (alk)₆ or B(OR)₃; e) Q′-LG; transition metal; f) H₂, Pd; g) PG deprotection; h) PG-N(R₃)(CH2)ₙCH₂CHO or PG-N(R₃)(CH₂)ₙCH₂CH₂-LG; NaBH(OAc)₃ or NaCHBH₃; i) PG deprotection.

### EXAMPLES

### Example 1

### 2-(4-(2-(2-methoxypyridin-3-yl)-3H-benzo[d]imidazol-5-yl)piperidin-1-yl)-N-methylethanamine

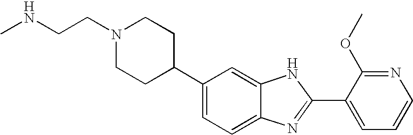 Part A: Preparation of tert-butyl methyl(2-oxoethyl)carbamate

Isobutyl chloroformate (7.1 mL, 55 mmol) was added drop wise to a stirred solution of Boc-sarcosine (9.5 g, 50 mmol) and N-methylmorphiline (12.1 mL, 110 mmol) in dichloromethane (100 mL) at −15° C. and the mixture was stirred at −15° C. for 15 min. N,O-dimethylhydroxylamine hydrochlororide (4.9 g, 50 mmol) was added and stirring was continued at −15° C. for 15 min, then at room temperature for 16 h. The mixture was washed with 0.2 M potassium bisulfate(2×50 mL), the organic layer separated and the aqueous layer extracted with dichloromethane (2×30 mL). The combined organic layers were dried over magnesium sulfate and the solvent evaporated to afford the Weinreb amide (11.9 g).

Lithium aluminum hydride (1M in THF, 62 mL) was added to a stirred solution of the above Weinreb amide in tetrahydrofuran (100 mL) at room temperature and the mixture was stirred for 20 min. The reaction was quenched with 0.2 M potassium bisulfate and extracted with ether (×3). The combined organic layer were washed with 2M HCl (×3), saturated sodium bicarbonate (×3). The organic layer was dried over magnesium sulfate and the solvent evaporated. The crude product was purified by flash column chromatography, eluting with 15% ethyl acetate/hexane to give the desired product as a colorless oil (3.1 g, 36% yield).

Part B: Preparation of tert-butyl 4-(trifluoromethylsul-fonyloxy)-5,6-dihydropyridine-1(2H)-carboxylate

To a solution of diisopropylamine (0.74 mL, 5.25 mmol) in tetrahydrofuran (20 mL), cooled to 0° C. was added n-butyllithium (2.5 M in hexanes, 2.1 mL, 5.25 mmol). After 15 min stirring, the reaction mixture was cooled to −78° C. and 1-tertbutoxycarbonyl-4-piperidone (1.0 g, 5.0 mmol) in tetrahydrofuran (5 mL) was added drop wise. After stirring for another hour, a solution of N-phenyltrifluoromethane sulfonimide (1.9 g, 5.25 mmol) in tetrahydrofuran (10 mL) was added. The reaction mixture was stirred at 0° C. for 3 h and quenched with saturated bicarbonate solution. The reaction was diluted with ether and washed with 15% potassium hydrogen sulfate, saturated bicarbonate solution, 1 N sodium hydroxide (×4), water (×2) and brine. The combined organic layers were dried over magnesium sulfate and concentrated in vacuo. Flash chromatography (silica gel, 10% ethyl acetate/hexane) gave the product as a colorless oil (1.1 g, 66%): 1H NMR (CDCl₃) δ 5.76 (br s, 1H), 4.04 (d, 2H, J=2.6 Hz), 3.63 (t, 2H, J=5.6 Hz), 1.48 (s, 9H), 2.44 (m, 2H).

Part C: Preparation of 2-nitro-4-(4,4,5,5-tetramethyl-1,3,2-dioxaborolan-2-yl)benzenamine

A mixture of 4-bromo-2-nitro-aniline (217 mg, 1 mmol), bis(pinacolato)diboron (279 mg, 1.1 mmol), [1,1′-bis(diphenylphosphino)ferrocene]dichloropalladium(II) (25 mg, 0.03 mmol) and potassium acetate (294 mg, 3 mmol) in methyl sulfoxide (4 mL) was heated under N₂ at 80° C. overnight. The crude reaction mixture was filtered through Celite and then partitioned between ethyl acetate and water. The organic layer was washed with saturated sodium bicarbonate (×3), dried over magnesium sulfate and concentrated in vacuo. Flash column chromatography (silica gel, 20% ethyl acetate/hexane) gave the desired product as a yellow solid (198 mg, 75% yield).

Part D: Preparation of tert-butyl 4-(4-amino-3-nitrophenyl)-5,6-dihydropyridine-1(2H)-carboxylate

A mixture of 2-nitro-4-(4,4,5,5-tetramethyl-1,3,2-dioxaborolan-2-yl)benzenamine (27 mg, 0.1 mmol), tert-butyl 4-(trifluoromethylsulfonyloxy)-5,6-dihydropyridine-1(2H)-carboxylate (36 mg, 0.11 mmol), [1,1′-bis(diphenylphosphino)ferrocene]dichloropalladium(II) (4.9 mg, 0.006 mmol) and potassium carbonate (31 mg, 0.23 mmol) in DMF (2 mL) was heated at 80° C. overnight. The crude reaction mixture was filtered through Celite and then partitioned between ethyl acetate and saturated sodium bicarbonate. The organic layer was washed with saturated sodium bicarbonate (×3), dried over magnesium sulfate and concentrated in vacuo. Flash column chromatography (silica gel, 5% ethyl acetate/hexane) gave the desired product (17.2 mg, 54% yield).

Part E: Preparation of 2-nitro-4-(1,2,3,6-tetrahydropyridin 4-yl)benzenamine

To tert-butyl 4-(4-amino-3-nitrophenyl)-5,6-dihydropyridine -1(2H)-carboxylate (100 mg, 3.2 mmol) was added trifluoroacetic acid in 1,2-dichloroethane (25%, 3 mL) and the reaction mixture was stirred at rt for 2 h. Solvent was evaporated and the residue was dissolved in methanol and was purified through a benzenesulfonic acid resin to give the free amine (69 mg, 100% yield).

Part F: Preparation of tert-butyl 2-(4-(4-amino-3-nitrophenyl)piperidin-1-yl)ethyl(methyl)carbamate

To a solution of 2-nitro-4-(1,2,3,6-tetrahydropyridin-4-yl)benzenamine (51 mg, 0.23 mmol) in 1,2-dichloroethane (10 mL) was added tert-butyl methyl(2-oxoethyl)carbamate (40 mg, 0.23 mmol), Sodium triacetoxyborohydride (69 mg, 0.33 mmol) and acetic acid (14 mg, 0.23 mmol). The resulting mixture was stirred at rt overnight. The reaction mixture was diluted with ethyl acetate and washed with saturated sodium bicarbonate (×3), dried over magnesium sulfate and concentrated in vacuo. Flash column chromatography (silica gel, 5% 2M ammonia/methanol/dichloromethane) gave the desired product (40 mg, 46% yield).

Part G: Preparation of tert-butyl 2-(4-(3,4-diaminophenyl)piperidin-1-yl)ethyl(methyl)carbamate

A mixture of tert-butyl 2-(4-(4-amino-3-nitrophenyl)piperidin-1-yl)ethyl(methyl)carbamate (40 mg, 0.11 mmol) and 10% palladium on carbon (12 mg) in methanol (25 mL) was hydrogenated at 50 psi for 2h. The reaction mixture was filtered through Celite and concentrated in vacuo to provide tert-butyl 2-(4-(3,4-diaminophenyl)piperidin-1-yl)ethyl(methyl)carbamate which was used without purification.

Part H: Preparation of 2-(4-(2-(2-methoxypyridin-3-yl)-3H-benzo[d]imidazol-5-yl)piperidin-1-yl)-N-methylethanamine (Example 1)

A mixture of tert-butyl 2-(4-(3,4-diaminophenyl)piperidin-1-yl)ethyl(methyl)carbamate (0.11 mmol) and 2-methoxynicotinaldehyde (15 mg, 0.11 mmol) in methanol (5 mL) was heated at 60° C. for 4h. solvent was evaporated and the crude intermediate was then treated with TFA in 1,2-dichloroethane (25%, 1 mL) at rt for 2 h. solvent was evaporated and the residue was purified by prep-HPLC to give 2-(4-(2-(2-methoxypyridin-3-yl)-3H-benzo[d]imidazol-5-yl)piperidin-1-yl)-N-methylethanamine as a brown oil (11 mg, 27% yield).

MS (ESI) m/z 366.22 (M+H) ¹H NMR (CD₃OD) δ 8.54 (dd, 1H, J=5.0, 1.7 Hz), 8.48 (dd, 1H, J=7.7, 1.7 Hz), 7.84 (d, 1H, J=8.1 Hz), 7.81 (s, 1H), 7.61 (dd, 1H, J=7.6, 1.1 Hz), 7.33 (dd, 1H, J=7.7,4.4 Hz), 4.25 (s, 3H), 3.80 (d, 2H,J=12.1 Hz), 3.58(s, 3H), 3.31 -3.15 (m, 3H), 2.82 (s, 3H), 2.32-2.17 (m, 4H).

### Example 2

### 2-(4-(2-(2-methoxypyridin-3-yl)-7-methyl-3H-benzo [d]-imidazol-5-yl)piperidin-1-yl)-N-methylethanamine

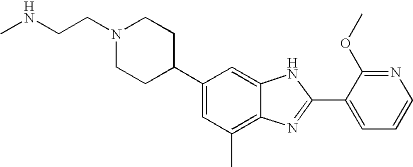 Part A: Preparation of 5-methyl-2-nitro-4-(4,4,5,5-tetramethyl-1,3,2-dioxaborolan-2-yl)benzenamine

A mixture of 4-bromo-5-methyl-2-nitro-aniline (217 mg, 1 mmol), bis(pinacolato)diboron (279 mg, 1.1 mmol), [1,1′-bis(diphenylphosphino)ferrocene]dichloropalladium(II)(25 mg, 0.03 mmol) and potassium acetate (294 mg, 3 mmol) in methyl sulfoxide (4 mL) was heated under N₂ at 80° C. overnight. The crude reaction mixture was filtered through Celite and then partitioned between ethyl acetate and water. The organic layer was washed with saturated sodium bicarbonate (×3), dried over magnesium sulfate and concentrated in vacuo. Flash column chromatography (silica gel, 20% ethyl acetate/hexane) gave the desired product as a yellow solid (198 mg, 75% yield)

Part B: Preparation of tert-butyl 4-(4-amino-3-5-methyl-nitrophenyl)-5,6-dihydropyridine-1(2H)-carboxylate

A mixture of 2-nitro-4-(4,4,5,5-tetramethyl-1,3,2-dioxaborolan-2-yl)benzenamine (27 mg, 0.1 mmol), tert-butyl 4-(trifluoromethylsulfonyloxy)-5,6-dihydropyridine-1(2H)-carboxylate (36 mg, 0.11 mmol), [1,1′-bis(diphenylphosphino)ferrocene]dichloropalladium(II) (4.9 mg, 0.006 mmol) and potassium carbonate (31 mg, 0.23 mmol) in DMF (2 mL) was heated at 80° C. overnight. The crude reaction mixture was filtered through Celite and then partitioned between ethyl acetate and saturated sodium bicarbonate. The organic layer was washed with saturated sodium bicarbonate (×3), dried over magnesium sulfate and concentrated in vacuo. Flash column chromatography (silica gel, 5% ethyl acetate/hexane) gave the desired product (17.2 mg, 54% yield).

Part C: Preparation of 5-methyl-2-nitro-4-(1,2,3,6-tetrahydropyridin-4-yl)benzenamine

To tert-butyl 4-(4-amino-5-methyl-3-nitrophenyl)-5,6-dihydropyridine-1(2H)-carboxylate (100 mg, 3.2 mmol) was added trifluoroacetic acid in 1,2-dichloroethane (25%, 3 mL) and the reaction mixture was stirred at rt for 2 h. Solvent was evaporated and the residue was dissolved in methanol and was purified through a benzenesulfonic acid resin to give the free amine (69 mg, 100% yield).

Part D: Preparation of tert-butyl 2-(4-(4-amino-5-methyl-3-nitrophenyl)piperidin-1-yl)ethyl(methyl)carbamate

To a solution of 5-methyl-2-nitro-4-(1,2,3,6-tetrahydropyridin-4-yl)benzenamine (51 mg, 0.23 mmol) in 1,2-dichloroethane (10 mL) was added tert-butyl methyl(2-oxoethyl)carbamate (40 mg, 0.23 mmol), Sodium triacetoxyborohydride (69 mg, 0.33 mmol) and acetic acid (14 mg, 0.23 mmol). The resulting mixture was stirred at rt overnight. The reaction mixture was diluted with ethyl acetate and washed with saturated sodium bicarbonate (×3), dried over magnesium sulfate and concentrated in vacuo. Flash column chromatography (silica gel, 5% 2M ammonia/methanol/dichloromethane) gave the desired product (40 mg, 46% yield).

Part E: Preparation of tert-butyl 2-(4-(3,4-diamino-5-methylphenyl)piperidin-1-yl)ethyl(methyl)carbamate

A mixture of tert-butyl 2-(4-(4-amino-5-methyl-3-nitrophenyl) piperidin-1-yl)ethyl(methyl)carbamate (40 mg, 0.11 mmol) and 10% palladium on carbon (12 mg) in methanol (25 mL) was hydrogenated at 50 psi for 2 h. The reaction mixture was filtered through Celite and concentrated in vacuo to provide tert-butyl 2-(4-(3,4-diamino-5-methylphenyl)piperidin-1-yl)ethyl(methyl)carbamate which was used without purification.

Part F: 2-(4-(2-(2-methoxypyridin-3-yl)-7-methyl-3H-benzo[d]-imidazol-5-yl)piperidin-1-yl)-N-methylethanamine (Example 2)

A mixture of tert-butyl 2-(4-(3,4-diamino-5-methylphenyl)piperidin-1-yl)ethyl(methyl)carbamate (0.11 mmol) and 2-methoxynicotinaldehyde (15 mg, 0.11 mmol) in methanol (5 mL) was heated at 60° C. for 4h. solvent was evaporated and the crude intermediate was then treated with TFA in 1,2-dichloroethane (25%, 1 mL) at rt for 2 h. solvent was evaporated and the residue was purified by prep-HPLC to give 2-(4-(2-(2-methoxypyridin-3-yl)-3H-benzo[d]imidazol-5-yl)piperidin-1-yl)-N-methylethanamine as a brown oil (11 mg, 27% yield).

MS (ESI) m/z 380.22 (M+H) ¹H NMR (CD₃OD) δ 8.54 (dd, 1H, J=5.0, 1.7 Hz), 8.48 (dd, 1H, J=7.7, 1.7 Hz), 7.84 (d, 1H, J=8.1 Hz), 7.81 (s, 1H), 7.61 (dd, 1H, J=7.6, 1.1 Hz), 7.33 (dd, 1H, J=7.7, 4.4 Hz), 4.25 (s, 3H), 3.80 (d, 2H, J=12.1 Hz), 3.58 (s, 3H), 3.31 -3.15 (m, 3H), 2.82 (s, 3H), 2.32-2.17 (m, 7H).

### Examples 3 to 97

The following examples were prepared using a method analogous to that used to prepare Example 2.

TABLE 1

| Example # | Structure | Mass (m/z) |
|---|---|---|
| 3 | 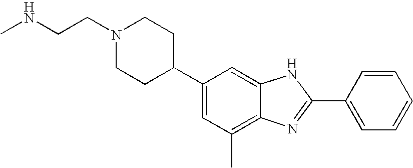 | 349.5 |
| 4 | 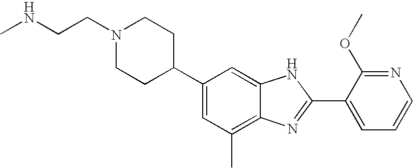 | 380.51 |
| 5 | 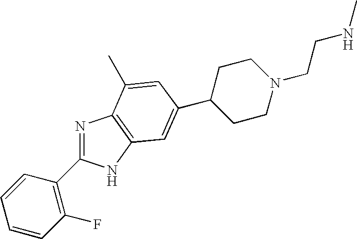 | 367.5 |
| 6 | 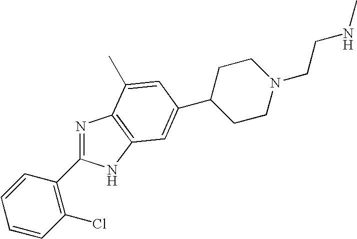 | 383.9 |
| 7 | 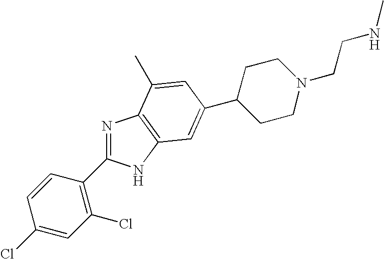 | 418.4 |
| 8 | 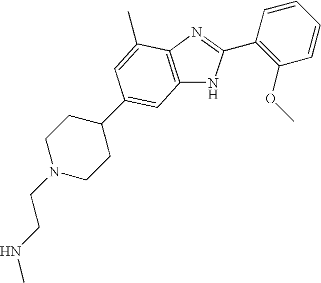 | 379.5 |
| 9 | 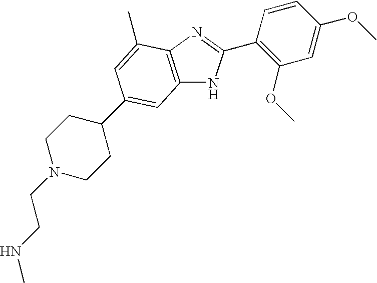 | 409.6 |
| 10 | 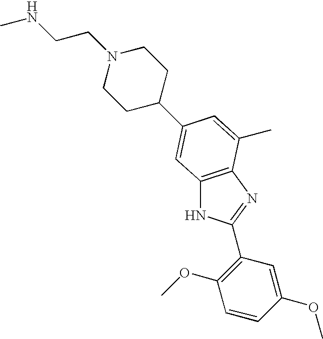 | 409.6 |
| 11 | 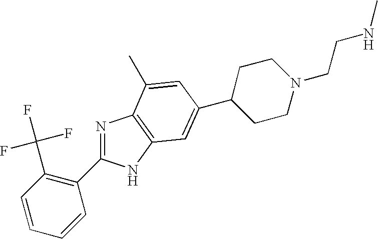 | 417.5 |
| 12 | 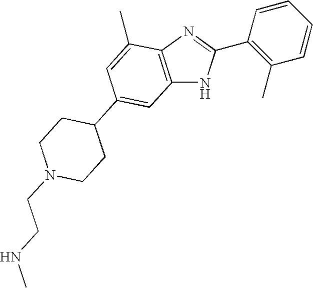 | 363.5 |
| 13 | 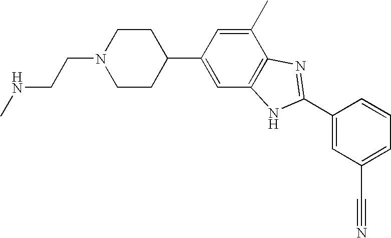 | 374.5 |
| 14 | 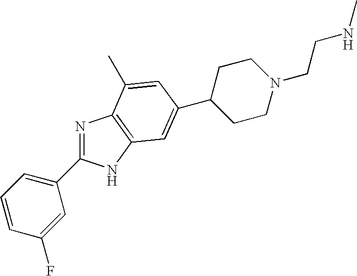 | 367.5 |
| 15 | 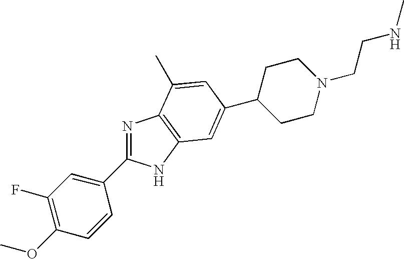 | 397.5 |
| 16 | 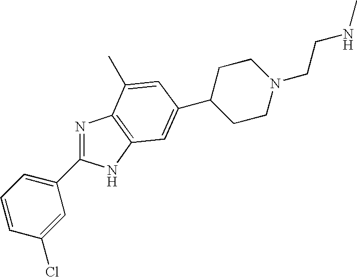 | 383.9 |
| 17 | 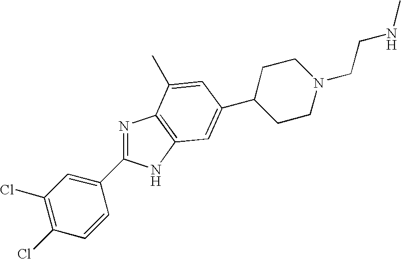 | 418.4 |
| 18 | 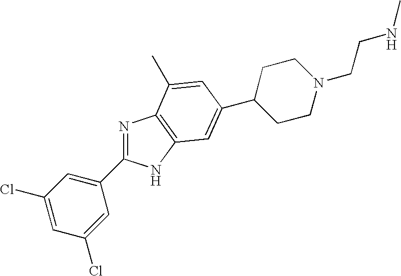 | 418.4 |
| 19 | 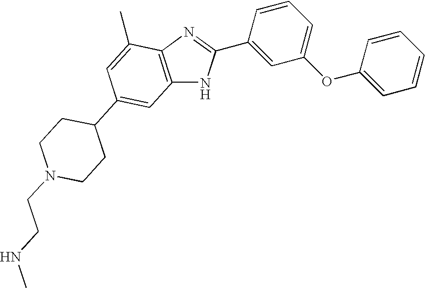 | 441.6 |
| 20 | 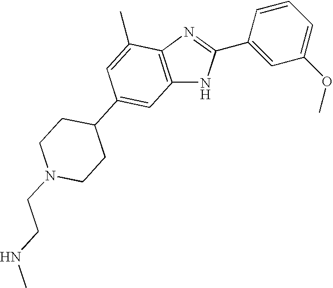 | 379.5 |
| 21 | 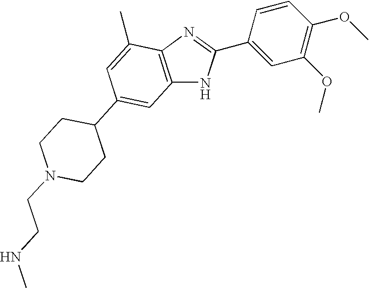 | 409.6 |
| 22 | 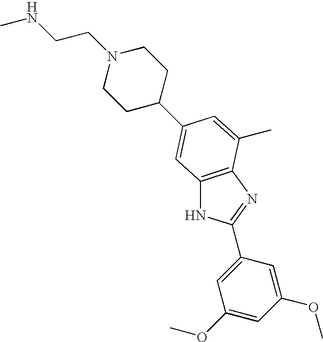 | 409.6 |
| 23 | 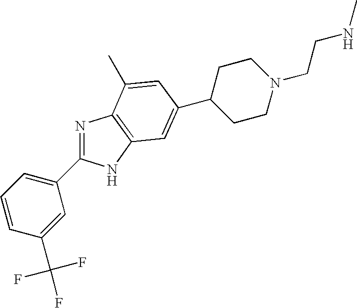 | 417.5 |
| 24 | 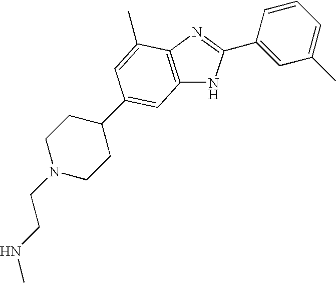 | 363.5 |
| 25 | 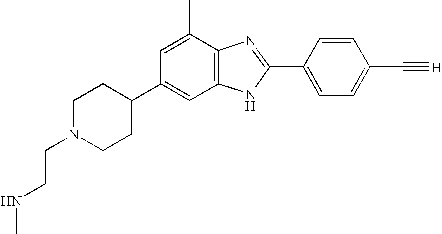 | 374.5 |
| 26 | 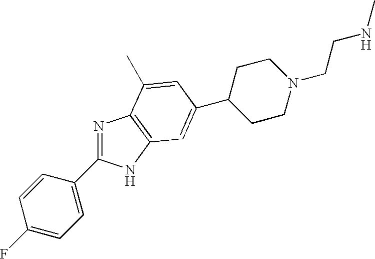 | 367.5 |
| 27 | 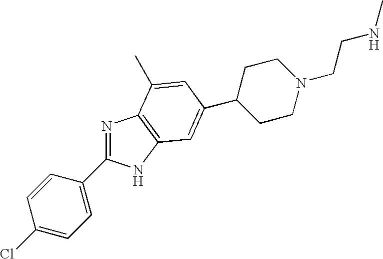 | 383.9 |
| 28 | 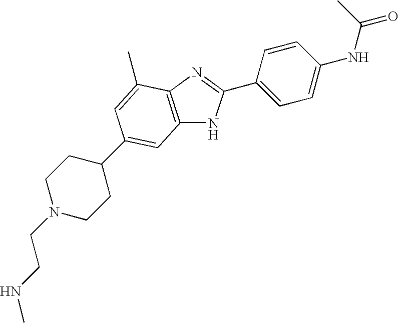 | 406.6 |
| 29 | 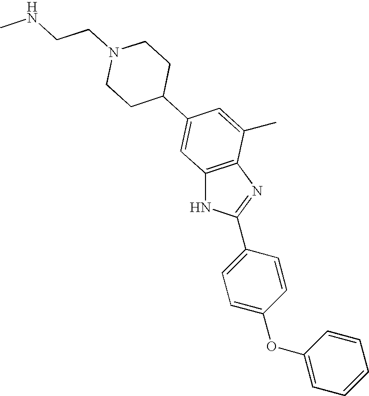 | 441.6 |
| 30 | 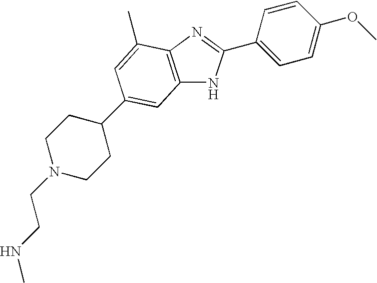 | 379.5 |
| 31 | 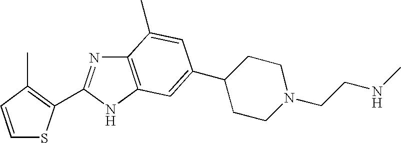 | 369.6 |
| 32 | 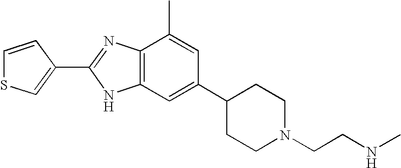 | 355.5 |
| 33 | 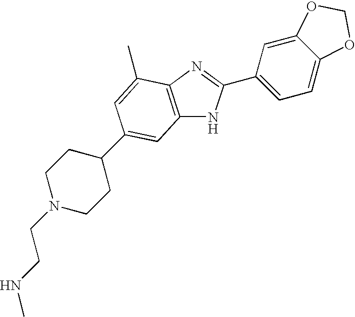 | 393.5 |
| 34 | 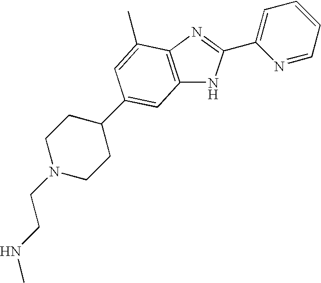 | 350.5 |
| 35 | 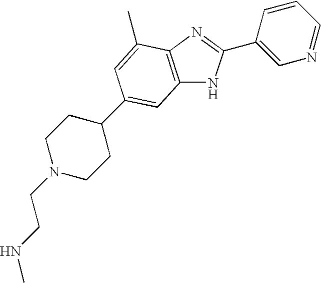 | 350.5 |
| 36 | 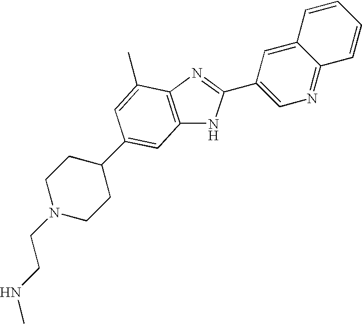 | 400.5 |
| 37 | 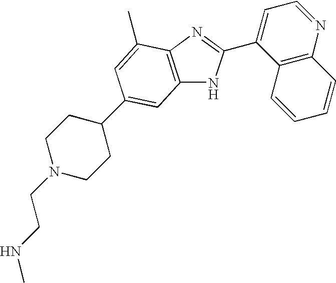 | 400.5 |
| 38 | 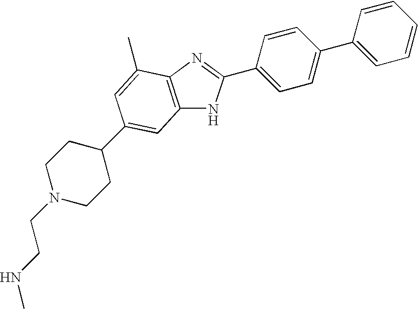 | 425.6 |
| 39 | 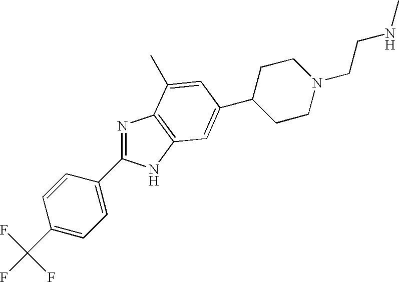 | 417.5 |
| 40 | 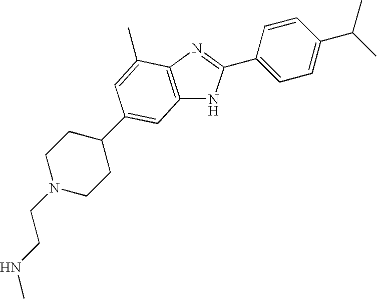 | 391.6 |
| 41 | 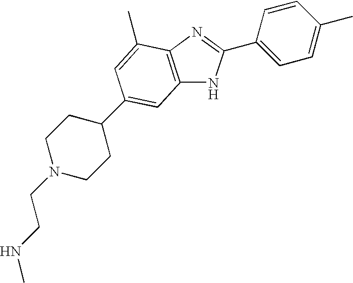 | 363.5 |
| 42 | 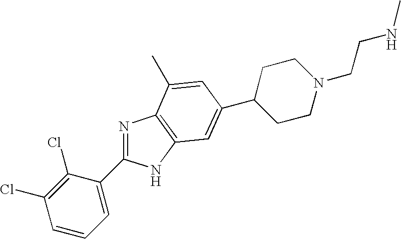 | 418.4 |
| 43 | 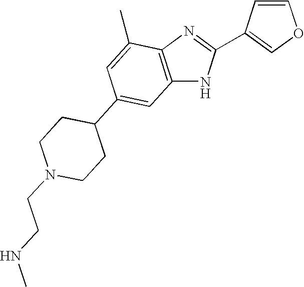 | 339.5 |
| 44 | 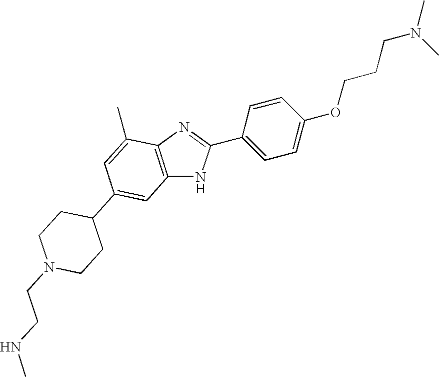 | 450.6 |
| 45 | 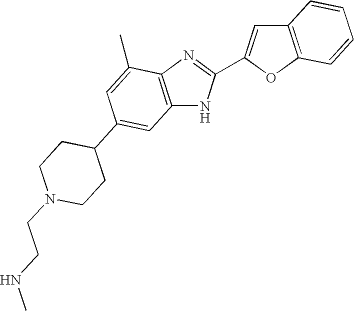 | 389.5 |
| 46 | 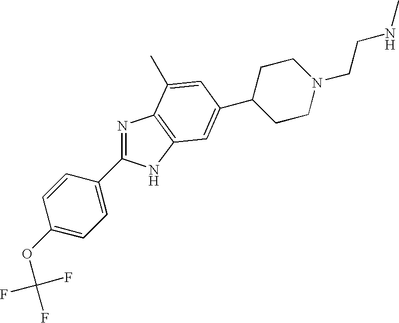 | 433.5 |
| 47 | 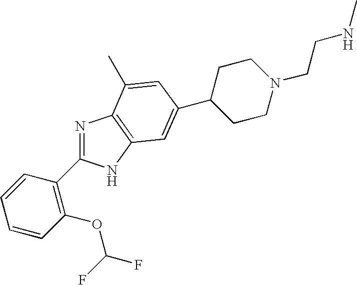 | 415.5 |
| 48 | 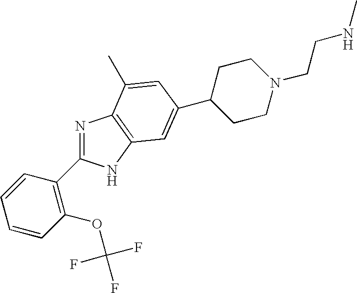 | 433.5 |
| 49 | 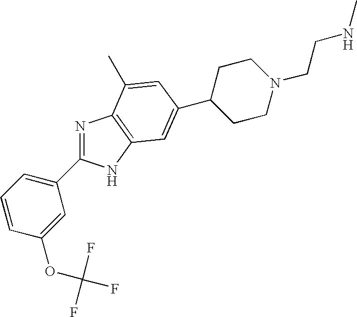 | 433.5 |
| 50 | 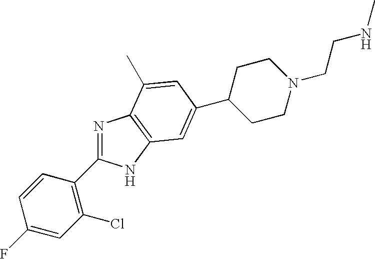 | 401.9 |
| 51 | 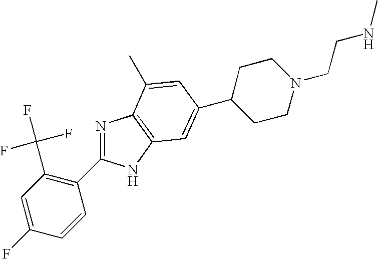 | 435.5 |
| 52 | 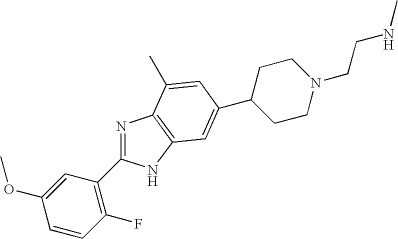 | 397.5 |
| 53 | 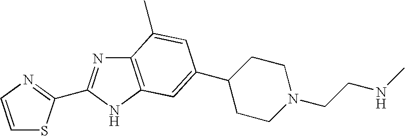 | 356.5 |
| 54 | 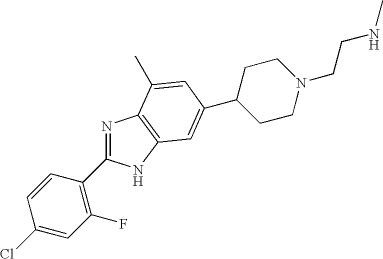 | 401.9 |
| 55 | 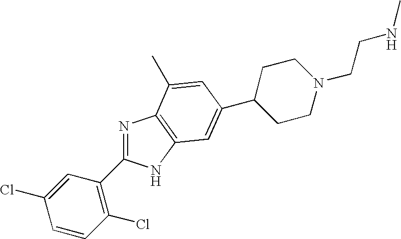 | 418.4 |
| 56 | 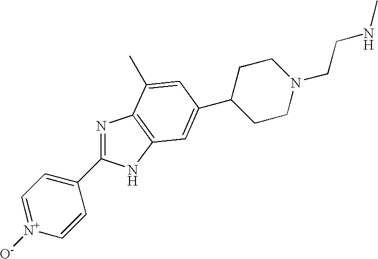 | 366.5 |
| 57 | 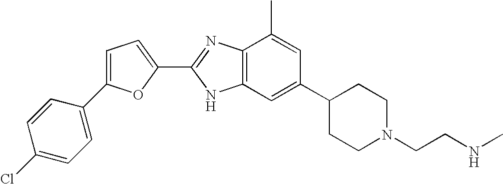 | 450 |
| 58 | 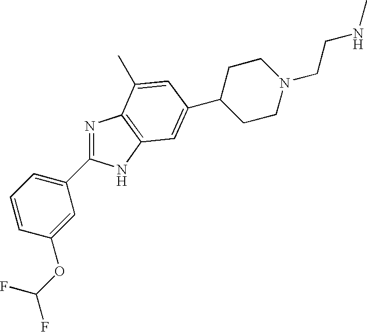 | 415.5 |
| 59 | 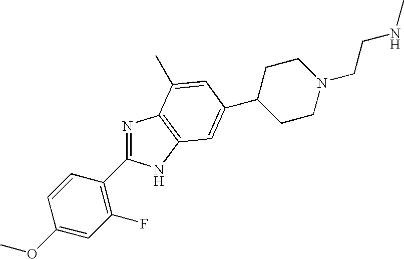 | 397.5 |
| 60 | 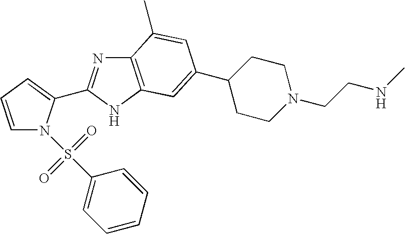 | 478.6 |
| 61 | 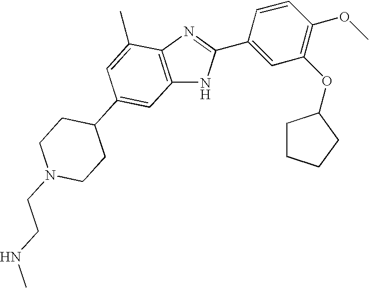 | 463.6 |
| 62 | 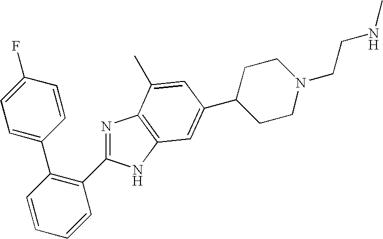 | 443.6 |
| 63 | 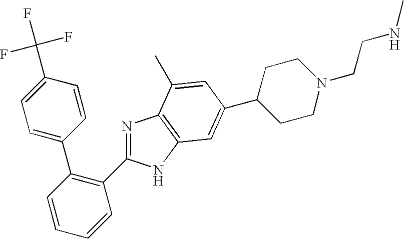 | 493.6 |
| 64 | 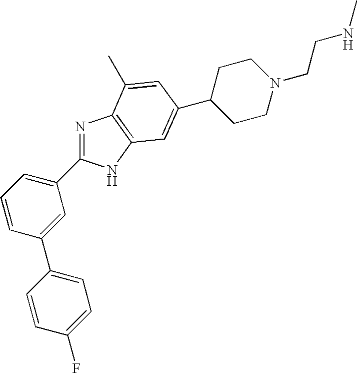 | 443.6 |
| 65 | 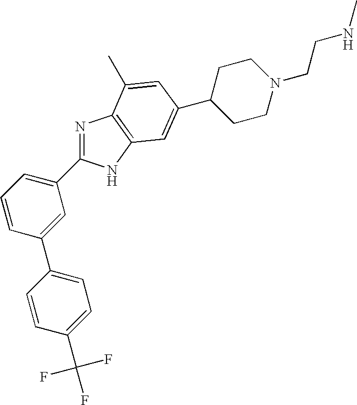 | 493.6 |
| 66 | 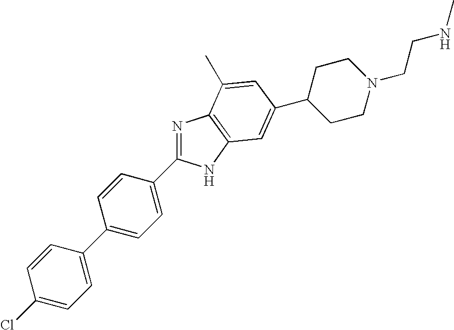 | 460 |
| 67 | 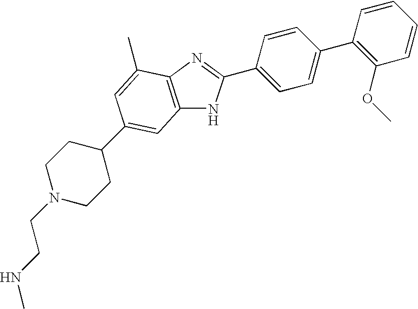 | 455.6 |
| 68 | 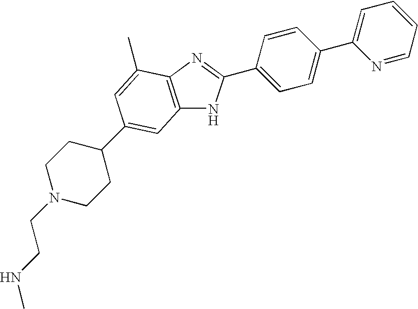 | 426.6 |
| 69 | 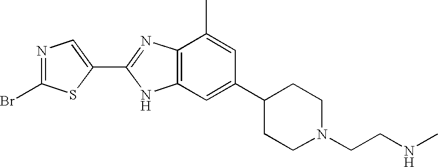 | 435.4 |
| 70 | 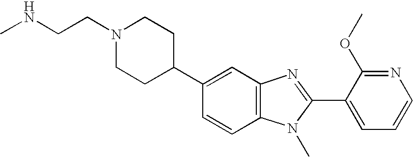 | 380.51 |
| 71 | 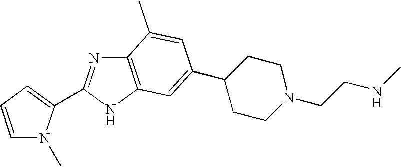 | 352.5 |
| 72 | 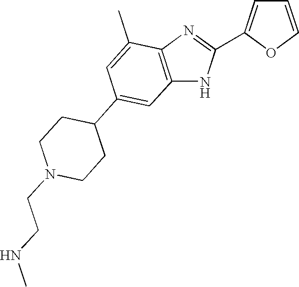 | 339.5 |
| 73 | 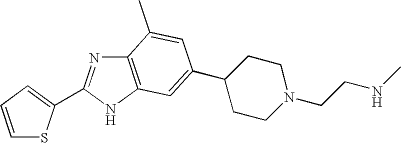 | 355.5 |
| 74 | 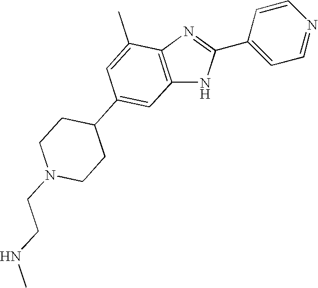 | 350.5 |
| 75 | 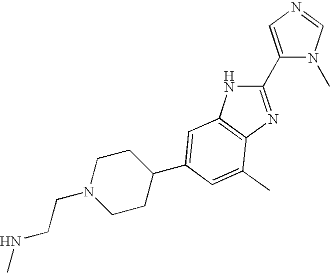 | 353.5 |
| 76 | 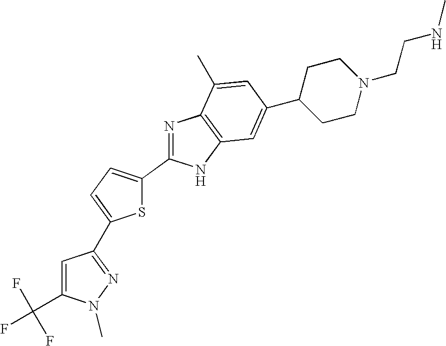 | 503.6 |
| 77 | 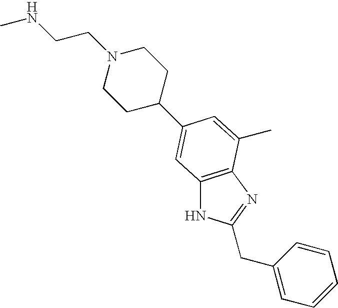 | 363.5 |
| 78 | 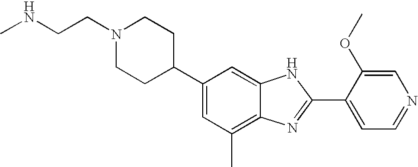 | 380.51 |
| 79 | 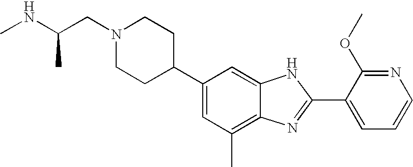 | 394.54 |
| 80 | 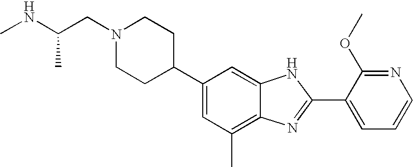 | 394.54 |
| 81 | 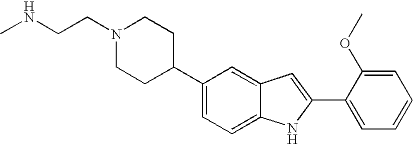 | 364.51 |
| 82 | 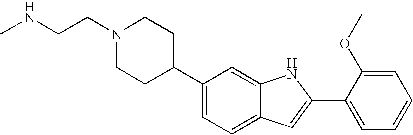 | 364.51 |
| 83 | 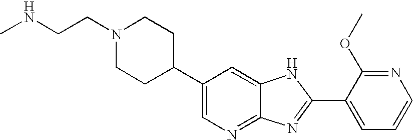 | 367.47 |
| 84 | 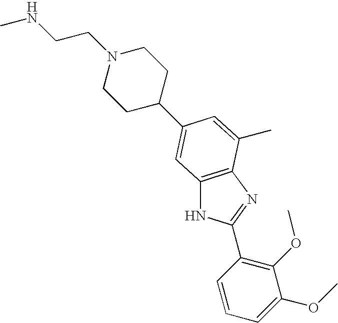 | 409.6 |
| 85 | 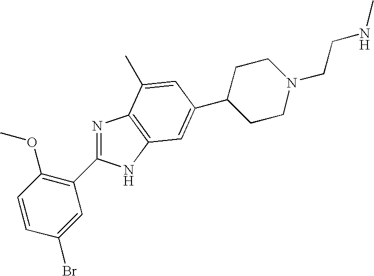 | 458.4 |
| 86 | 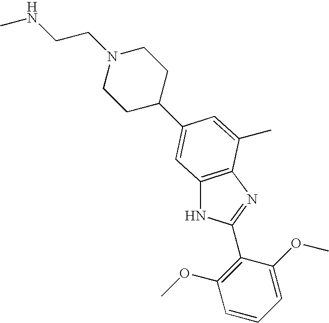 | 409.6 |
| 87 | 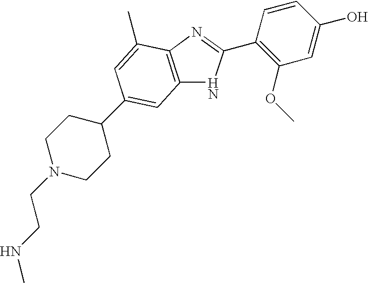 | 395.5 |
| 88 | 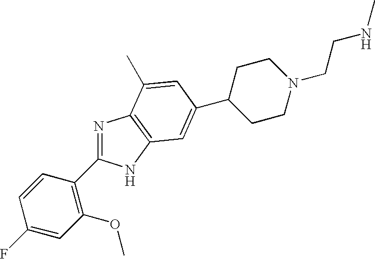 | 397.5 |
| 89 | 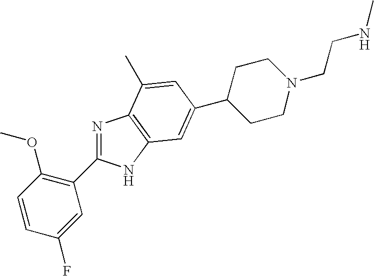 | 397.5 |
| 90 | 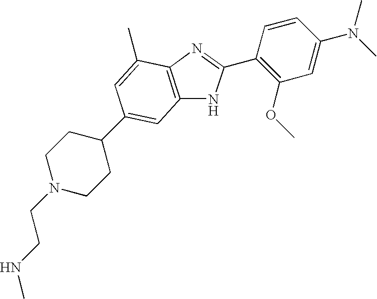 | 422.6 |
| 91 | 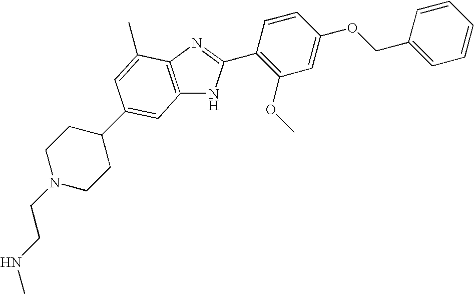 | 485.6 |
| 92 | 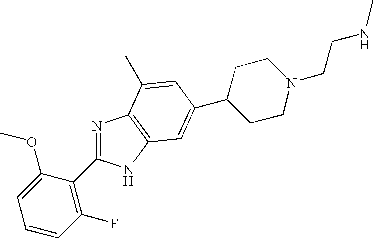 | 397.5 |
| 93 | 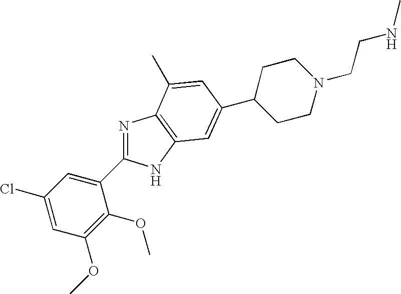 | 444 |
| 94 | 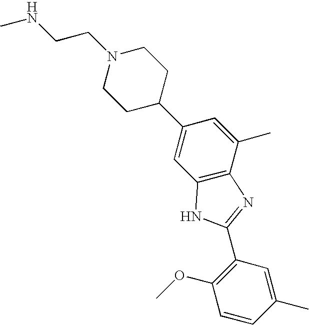 | 393.6 |
| 95 | 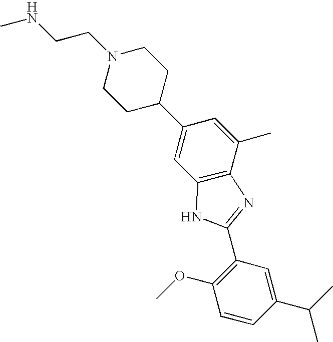 | 421.6 |
| 96 | 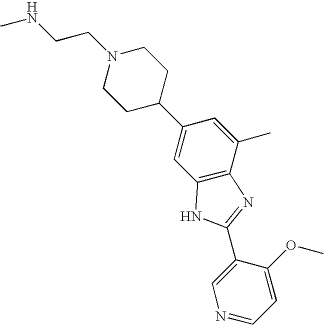 | 380.5 |
| 97 | 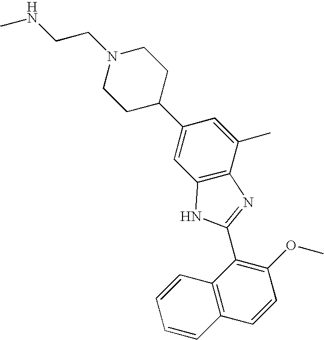 | 429.6 |

### Example 98

### 2-(4-(2-(2-methoxyphenyl)-1H-indol-5-yl)piperidin-1-yl)-N-methylethanamine

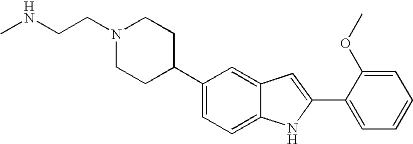 Part A: Preparation of 4-bromo-2-(2-(2-methoxyphenyl)ethynyl)aniline

A mixture of 4-bromo-2-iodoaniline (1.2 g, 4.0 mmol), 2-ethynylanisol (0.46 g, 4.0 mmol) dichloro-bis(triphenyl-phosphine)palladium (72 mg, 0.08 mmol), copper(l) iodide (7.6 mg, 0.04 mmol) in triethylamine (12 mL) was heated under nitrogen at 55° C. for 6 h. The solid was filtered off and the filtrate was concentrated in vacuo. The crude product was purified by flash column chromatography (silica gel, 10% ethyl acetate/hexane) to afford 4-bromo-2-(2-(2-methoxyphenyl)ethynyl)aniline as a light brown solid (0.97 g, 80% yield).

MS (ESI) m/z 302 (M+H) ¹H NMR (CDCl₃) δ 7.45-7.48 (m, 2H), 7.30-7.34 (m, 1H), 7.20 (dd, 1H, J=8.2, 2.2 Hz), 6.96 (t, 1H, J=8.3 Hz), 6.92 (d, 1H, J=8.3 Hz), 6.61 (d, 1H, J=8.8 Hz), 4.52 (s, 2H), 3.92 (s, 3H).

Part B: Preparation of 5-bromo-2-(2-methoxyphenyl)-1H-indole

A mixture of 4-bromo-2-(2-(2-methoxyphenyl)ethynyl)aniline (0.87 g, 2.9 mmol) and palladium dichloride (25 mg, 0.14 mmol) in acetonitrile (15 mL) was heated at 75° C. for 4 h. The reaction mixture was filtered through a plug of Celite and concentrated. Purification with flash column chromatography (silica gel, 1:1 dichloromethane/hexane) afforded 5-bromo-2-(2-methoxyphenyl)-1H-indole as a white solid (0.79 g, 90% yield).

MS (ESI) m/z 302 (M+H) ¹H NMR (CDCl₃) δ 9.72 (s, 1H), 7.82 (dd, 1H, J=7.9, 1.8 Hz), 7.74 (m, 1H), 7.21-7.34 (m, 4H), 7.02-7.09 (m, 2H), 6.82 (d, 1H, J=2.2 Hz), 4.03 ( s, 3H).

Part C: Preparation of 2-(2-methoxyphenyl)-5-(4,4,5,5-tetramethyl-1,3,2-dioxaborolan-2-yl)-1H-indole

A mixture of 5-bromo-2-(2-methoxyphenyl)-1H-indole (1.51 g, 5.0 mmol), bis(pinacolato)diboron (1.91 g, 7.5 mmol), [1,1′-bsi(diphenylphosphino)ferrocene]dichloropalladium(II) (PdCl2dppf) (0.24 g, 0.3 mmol), potassium acetate (1.47 g, 15.0 mmol) in DMSO (20 mL) was heated at 110° C. under nitrogen overnight. The reaction mixture was partitioned between ethyl acetate and water. The organic layer was washed with brine 3 times, dried over magnesium sulfate and concentrated in vacuo. Flash column chromatography of the crude (silica gel, 8% ethyl acetate/hexane) gave 2-(2-methoxyphenyl)-5-(4,4,5,5-tetramethyl-1,3,2-dioxaborolan-2-yl)-1H-indole (0.84 g, 48% yield).

MS (ESI) m/z 350 ¹H NMR (CDCl₃) δ 9.73 (s, 1H), 8.16 (s, 1H), 7.84 (dd, 1H, J=7.7, 1.2 Hz), 7.62 (dd, 1H, J=8.3, 1.1 Hz), 7.40 (d, 1H, J=8.3 Hz), 7.24-7.29 (m, 1H), 6.99-7.07 (m, 2H), 6.90 (s, 1H), 4.00 (s, 3H), 1.37 (s, 12H).

Part D: Preparation of teff-butyl 4-(2-(2-methoxyphenyl)-1H-indol-5-yl)-5,6-dihydropyridine-1(2H)-carboxylate

To a nitrogen flashed flask charged with 2-(2-methoxyphenyl)-5-(4,4,5,5-tetramethyl-1,3,2-dioxaborolan-2-yl)-1H-indole (800 mg, 2.3 mmol), cesium carbonate (2.24 g, 6.9 mmol) and [1,1′-bsi(diphenylphosphino)ferrocene]dichloropalladium(II) (112 mg, 0.14 mmol) was added a solution of tert-butyl 4-(trifluoromethylsulfonyloxy)-5,6-dihydropyridine-1(2H)-carboxylate (1.5 g, 4.6 mmol) in DMF (12 mL). The mixture was heated at 90° C. under nitrogen overnight. The reaction mixture was partitioned between ethyl acetate and water. The organic layer was washed with saturated sodium bicarbonate 3 times, dried over magnesium sulfate and concentrated in vacuo. The crude was purified with flash column chromatography (silica gel, 20% ethyl acetate/hexane) to give tert-butyl 4-(2-(2-methoxyphenyl)-1H-indol-5-yl)-5,6-dihydropyridine-1(2H)-carboxylate (0.51 g, 87% yield based on reacted material) along with 0.29 g of recovered starting material.

MS (ESI) m/z 405 ¹H NMR (CDCl₃) δ 9.66 (s, 1H), 7.82 (dd, 1H, J=7.5, 1.3 Hz), 7.60 (s, 1H), 7.35 (d, 1H, J=8.8 Hz), 7.22-7.29(m, 2H), 7.00-7.07 (m, 2H), 6.87 (d, 1H J=1.1 Hz), 6.01 (s, 1H), 4.09 (br s, 2H), 4.00 (s, 3H), 3.67 (t, 2H, J=5.7 Hz), 2.62 (br s, 2H), 1.50 (s, 9H).

Part E: Preparation of tert-butyl 2-(4-(2-(2-methoxyphenyl)-1H-indol-5-yl)-5,6-dihydropyridin-1(2H)-yl)ethyl(methyl)carbamate

Tert-butyl 4-(2-(2-methoxyphenyl)-1H-indol-5-yl)-5,6-dihydropyridine-1(2H)-carboxylate (306 mg, 0.76 mmol) was treated with TFA in dichloroethane (25%, 3 mL) at rt for 1 h. Solvent was evaporated and the residue was filtered through a SCX cartridge and eluted with 2N ammonia in methanol to give the intermediate as a light brown oil.

To the above intermediate in dichloroethane (10 mL) was added tert-butyl methyl(2-oxoethyl)carbamate (131 mg, 0.76 mmol), (polystyrylmethyl)triethylamonium cyanoboro-hydride (4.1 mmol/g, 277 mg, 1.1 mmol) and acetic acid (45 mg, 0.76 mmol). The reaction mixture was stirred at rt for 4 h. The resin was filtered and the filtrate was concentrated in vacuo. Purification with flash column chromatography (silica gel, 4% 2N ammonia in methanol/dichloromethane) afforded tert-butyl 2-(4-(2-(2-methoxyphenyl)-1H-indol-5-yl)-5,6-dihydropyridin-1(2H)-yl)ethyl(methyl)carbamate as a colorless oil (224 mg, 64% yield).

MS (ESI) m/z 462 (M+H) ¹H NMR (CDCl₃) δ 9.65 (s, 1H), 7.83 (dd, 1H, J=7.9, 1.8 Hz), 7.61 (s, 1H), 7.22-7.36 (m, 2H), 7.01-7.08 (m, 2H), 6.87 (d, 1H, J=1.8 Hz), 6.04 (m, 1H), 4.01 (s, 3H), 3.49 (br s, 2H), 3.24 (br s, 2H), 2.91 (s, 3H), 2.79 (m, 2H), 2.58-2.70 (m, 2H).

Part F: Preparation of 2-(4-(2-(2-methoxyphenyl)-1H-indol-5-yl)piperidin-1-yl)-N-methylethanamnine

A mixture of tert-butyl 2-(4-(2-(2-methoxyphenyl)-1H-indol-5-yl)-5,6-dihydropyridin-1 (2H)-yl)ethyl(methyl)carbamate (192 mg, 0.42 mmol) and palladium on carbon (10%, 44 mg) in methanol (30 mL) was hydrogenated at 30 psi for 6 h. The palladium catalyst was filtered through a plug of Celite and the filtrate was concentrated and then treated with TFA (25% in dichloroethane, 4.0 mL) at rt for 1 h. The reaction mixture was diluted with dichloromethane and washed with saturated sodium bicarbonate 3 times, dried over magnesium sulfate. Solvent was evaporated and the crude product was purified by prep-HPLC. The product from prep-HPLC was neutralized by dissolving in dichloromethane and washed with saturated sodium bicarbonate, yielding 2-(4-(2-(2-methoxyphenyl)-1H-indol-5-yl)piperidin-1-yl)-N-methylethan-amine (Example 98) as a yellow foaming solid (53 mg, 35%).

MS (ESI) m/z 364 (M+H) ¹H NMR (CDCl₃) δ 9.58 (s, 1H), 7.81 (dd, 1H, J=7.7, 1.5 Hz), 7.46 (s, 1H), 7.32 (d, 1H, J=8.4 Hz), 7.22-7.29 (m, 2H), 6.98-7.07 (m, 3H), 6.83 (d, 1H, J=1.5 Hz), 3.99 (s, 3H), 3.04 (m, 2H), 2.73 (t, 2H, J=6.6 Hz), 2.52-2.63 (m, 3H), 2.48 (s, 3H), 2.07-2.15 (m, 4H), 1.78-1.90 (m, 4H). LC/MS m/z 364.

### Example 99

### 2-(4-(3-fluoro-2-(2-methoxyphenyl)-1H-indol-5-yl)piperidin-1-yl)-N-methylethanamine

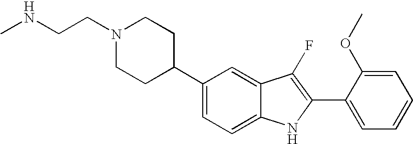 Method 1:
Part A: Preparation of 5-bromo-3-fluoro-2-(2-methoxyphenyl)-1H-indole

5-bromo-2-(2-methoxyphenyl)-1H-indole (prepared using procedure provided for Part B of example 98) was dissolved in a 1 to 1 mixture of methyl sulfoxide and acetonitrile (6 mL). The solution was cooled to 0° C. and the fluorinating reagent, 1-(chloromethyl)-4-fluoro-1,4-diazoniabicyclo[2.2.2]octane ditetrafluoroborate (SelectFluor, 0.25 equiv., 110 mg, 0.31 mmol) was added. After addition the cooling bath was removed and the reaction mixture was stirred at room temperature for 30 minutes. The fluorinating reagent was continually added in this fashion until half of the starting material was consumed (1 equivalent of SelectFluor was added). The reaction mixture was partitioned between ethyl acetate and water and the two layers were separated. The organic layer was washed with water, dried over MgSO4 and concentrated, the crude product was purified by flash column chromatography, eluting with 50% chloroform/Hexane to give 5-bromo-3-fluoro-2-(2-methoxyphenyl)-1H-indole(0.16 g, 40% yield).

¹H NMR (CDCl₃) δ=9.21 (s, 1H), 7.97 (dd, J=7.7, 1.7 Hz, 1H), 7.68 (s, 1H), 7.11-7.26 (m, 3H), 7.04 (t, J=7.7 Hz, 1H), 6.97 (d, J=8.3 Hz), 3.94 (s, 3H).

Part B: Preparation of 3-fluoro-2-(2-methoxyphenyl)-5-(4,4,5,5-tetramethyl-1,3,2-dioxaborolan-2-yl)-1H-indole

A mixture of 5-bromo-3-fluoro-2-(2-methoxyphenyl)-1H-indole (0.16 g, 0.48 mmol), bis(pinacolato)diboron (0.27 g, 0.97 mmol), [1,1′-bis(diphenylphosphino)ferrocene]dichloro palladium (20 mg, 0.02 mmol) and potassium acetate (142 mg, 1.45 mmol) in methyl sulfoxide (3 mL) was heated at 80° C. under N2 overnight, the reaction mixture was partitioned between EtOAc and water. The organic layer was washed 3 times with saturated sodium bicarbonate, dried over MgSO₄ and concentrated, the crude product was used without purification (150 mg, 84% yield).

Part C: Preparation of tert-butyl 4-(3-fluoro-2-(2-methoxyphenyl)-1H-indol-5-yl)-5,6-dihydropyridine-1(2H)-carboxylate

A mixture of 3-fluoro-2-(2-methoxyphenyl)-5-(4,4,5,5-tetramethyl-1,3,2-dioxaborolan-2-yl)-1H-indole (0.15 g, 0.41 mmol), tert-butyl 4-(trifluoromethylsulfonyloxy)-5,6-dihydropyridine-1(2H)-carboxylate (270 mg, 0.82 mmol), [1,1′-bis(diphenylphosphino)ferrocene]dichloropalladium (20 mg, 0.02 mmol) and Cesium carbonate (0.40 g, 1.2 mmol) in DMF (6 mL) was heated at 100° C. under N2 overnight. The reaction mixture was partitioned between EtOAc and water, The organic layer was washed with saturated sodium bicarbonate (3×), dried over MgSO₄ and concentrated, purification by flash column chromatography, eluting with 10% EA/Hexane, afforded the tert-butyl 4-(3-fluoro-2-(2-methoxyphenyl)-1H-indol-5-yl)-5,6-dihydropyridine-1(2H)-carboxylate as an colorless oil (0.12 g, 70% yield).

Part D: Preparation of tert-butyl 2-(4-(3-fluoro-2-(2-methoxyphenyl)-1H-indol-5-yl)-5,6-dihydropyridin-1(2H)-yl)ethyl(methyl)carbamate

Tert-butyl 4-(3-fluoro-2-(2-methoxyphenyl)-1H-indol-5-yl)-5,6-dihydropyridine-1(2H)-carboxylate (0.12 g, 0.28 mmol) was treated with 25% TFA in dichloroethane at room temperature for 1 h. solvent was evaporated and the residue was purified with SCX cartridge.

To the above intermediate in dichloroethane (20 mL) was added sodium triacetoxyboronhydride (84 mg, 0.40 mmol), and acetic acid (17 mg, 0.28 mmol) and the reaction mixture was stirred at rt overnight. after aqueous workup, the organic layer was dried and concentrated. The crude was purified by flash column chromatography, eluting with 3% 2M ammonia/methanol/dichloromethane to give the tert-butyl 2-(4-(3-fluoro-2-(2-methoxyphenyl)-1H-indol-5-yl)-5,6-dihydropyridin-1(2H)-yl)ethyl(methyl)carbamate (60 mg, 44% yield).

Part E: Preparation of 2-(4-(3-fluoro-2-(2-methoxyphenyl)-1H-indol-5-yl)piperidin-1-yl)-N-methylethanamine

Tert-butyl 2-(4-(3-fluoro-2-(2-methoxyphenyl)-1H-indol-5-yl)-5,6-dihydropyridin-1(2H)-yl)ethyl(methyl)carbamate (60 mg, 0.13 mmol) and 10% palladium on carbon (18 mg) in methanol (20 mL) was hydrogenated at 30 psi at room temperature for 3 hours. The reaction mixture was filtered through a pad of Celite and the filtrate was concentrated in vacuo to give a oil which was treated with 25% TFA in dichloroethane (3 mL) at rt for 0.5 h, solvent was evaporated and the residue was purified using preparative HPLC using conditions outlined below to afford 2-(4-(3-fluoro-2-(2-methoxyphenyl)-1H-indol-5-yl)piperidin-1-yl)-N-methylethanamine (Example 99) (19 mg, 38% yield).

Conditions: Column—YMC ODS (20×50 mm)

Solvents—A-90% water-10% methanol-0.1% TFA

B-10% water-90% methanol-0.1% TFA

Gradient—25% B to 100% B in 12 min

Retention time: 9 min

MS (ESI) m/z 382 (M+H) ¹H NMR (CD₃OD) δ=7.80 (dd, J=9.4, 1.7 Hz, 1H), 7.29-7.42 (m, 3H), 7.02-7.14 (m, 3H), 3.96 (s, 3H), 3.70-3.78 (m, 2H), 3.56 (s, 4H), 3.19-3.30 (m, 2H), 2.95-3.04 (m, 1H), 2.82 (s, 3H), 2.10-2.22 (m, 4H).

Method 2:

Part A: Preparation of tert-butyl 4-(4-amino-3-iodophenyl)piperidine-1-carboxylate

Iodomonochloride (3.2 g, 20 mmol) in methanol (10 ml) was added slowly to a suspension of tert-butyl 4-(4-aminophenyl)piperidine-1-carboxylate (5.0 g, 18.1 mmol) and calcium carbonate (2.7 g, 27 mmol) in a mixed solvent of methanol (40 ml) and water (20 ml) at room temperature. The reaction mixture was stirred at room temperature for half an hour. Methanol was evaporated and the residual suspension was partitioned between ethyl acetate and water. The layers were separated and the organic layer was washed with saturated sodium bicarbonate 2 times, dried over magnesium sulfate and concentrated in vacuo. The crude product (6.6 g) was used in the next step without purification.

MS (ESI) m/z 403 (M+H).

Part B: Preparation of tert-butyl 4-(4-amino-3-((2-methoxyphenyl)ethynyl)phenyl)piperidine-1-carboxylate

A mixture of tert-butyl 4-(4-amino-3-iodophenyl)piperidine-1-carboxylate (6.6 g, 16.4 mmol), 2-ethynylanisole (2.2 g, 16.4 mmol), dichlorobistriphenylphosphinepalladium(II) (0.23 g, 0.33 mmol) and copper(I) iodide (32 mg, 0.16 mmol) in triethylamine (50 ml) was heated at 55° C. under nitrogen for 6 h. Solvent was evaporated and the crude was purified by column chromatography, eluting with 10% ethyl acetate/hexanes to give a light brown solid (5.3 g, 80% yield).

MS (ESI) m/z 407 (M+H).

Part C: Preparation of tert-butyl 4-(2-(2-methoxyphenyl)-1H-indol-5-yl)piperidine-1-carboxylate

A mixture of tert-butyl 4-(4-amino-3-((2-methoxyphenyl)ethynyl)phenyl)piperidine-1-carboxylate (5.3 g, 13 mmol) and palladium chloride (116 mg, 0.65 mmol) in acetonitrile (50 ml) was heated at 80° C. overnight. Solvent was evaporated and the residual was purified by column chromatography, eluting with 30% ethyl acetate/hexanes (5.3 g, 100% yield).

MS (ESI) m/z 407 (M+H).

Part D: Preparation of tert-butyl 2-(4-(2-(2-methoxyphenyl)-1H-indol-5-yl)piperidin-1-yl)ethyl(methyl)carbamate

Tert-butyl 4-(2-(2-methoxyphenyl)-1H-indol-5-yl)piperidine-1-carboxylate (5.3 g, 13.0 mmol) was treated with 25% TFA in dichloromethane (10 ml) at room temperature for 1 h. solvent was evaporated and the residue was purified by column chromatography, eluting with 8% 2N ammonia/methanol/dichloromethane to give the intermediate 2-(2-methoxyphenyl)-5-(piperidin-4-yl)-1H-indole as a brown oil.

A mixture of the above intermediate, tert-butyl methyl(2-oxoethyl)carbamate (2.7 g, 15.7 mmol), sodium triacetoxyboronhydride (3.9 g, 18.3 mmol), and acetic acid (0.78 g, 13.0 mmol) in dichloromethane (100 ml) was stirred at room temperature overnight. The reaction mixture was diluted with ethyl acetate and washed 3 times with saturated sodium bicarbonate. The organic layer was dried over magnesium sulfate and concentrated in vacuo. Purification by flash column chromatography, eluting with 3% 2N ammonia/methanol/dichloromethane afforded the desired product (4.9 g, 81% yield).

MS (ESI) m/z 464 (M+H).

Part E: Preparation of tert-butyl 2-(4-(3-fluoro-2-(2-methoxyphenyl)-1H-indol-5-yl)piperidin-1-yl)ethyl(methyl)carbamate

Tert-butyl 2-(4-(2-(2-methoxyphenyl)-1H-indol-5-yl)piperidin-1-yl)ethyl(methyl)carbamate (2.2 g, 4.8 mmol) was dissolved in a 1 to 1 mixture of methyl sulfoxide and acetonitrile (80 ml). The solution was cooled to 0° C. and the fluorinating reagent, 1-(chloromethyl)-4-fluoro-1,4-diazoniabicyclo[2.2.2]octane ditetrafluoroborate (SelectFluor, 0.25 equiv., 0.5 g, 1.4 mmol) was added. After addition the cooling bath was removed and the reaction mixture was stirred at room temperature for 30 minutes. The fluorinating reagent was continually added in this fashion until half of the starting material was consumed (1.2 equivalent of SelectFluor was added). The reaction mixture was partitioned between ethyl acetate and water and the two layers were separated. The organic layer was washed with saturated sodium bicarbonate, dried over magnesium sulfate and concentrated in vacuo, the crude product was purified by preparative HPLC (45-80% gradient) to afford the desired product (0.88 g, 38% yield).

MS (ESI) m/z 482 (M+H).

Part F: Preparation of 2-(4-(3-fluoro-2-(2-methoxyphenyl)-1H-indol-5-yl)piperidin-1-yl)-N-methylethanamine

Tert-butyl 2-(4-(3-fluoro-2-(2-methoxyphenyl)-1H-indol-5-yl)piperidin-1-yl)ethyl(methyl)carbamate (0.88 g, 1.8 mmol) was treated with 25% TFA in dichloroethane (20 ml) at room temperature for 0.5 h, solvent was evaporated and the residue was purified by preparative HPLC to afford the final product (0.49 g, 71% yield).

MS (ESI) m/z 382 (M+H) ¹H NMR (CD₃OD) δ=7.80 (dd, J=9.4, 1.7 Hz, 1H), 7.29-7.42 (m, 3H), 7.02-7.14 (m, 3H), 3.96 (s, 3H), 3.70-3.78 (m, 2H), 3.56 (s, 4H), 3.19-3.30 (m, 2H), 2.95-3.04 (m, 1H), 2.82 (s, 3H), 2.10-2.22 (m, 4H).

Method 3:

Part A: Preparation of tert-butyl 2-(4-hydroxypiperidin-1-yl)ethyl(methyl)carbamate

A mixture of piperidin-4-ol (5.05 g, 50 mmol), tert-butyl methyl(2-oxoethyl)carbamate (8.7 g, 50 mmol), sodium triacetoxyboronhydride (14.8 g, 70 mmol), and acetic acid (3.0 g, 50 mmol) in dichloromethane (250 ml) was stirred at room temperature overnight. The reaction mixture was concentrated in vacuo and dissolved in ethyl acetate (150 ml) and washed 3 times with saturated sodium bicarbonate. The organic layer was dried over magnesium sulfate and concentrated in vacuo to give 9.9 g of crude product which was used without purification.

Part B: Preparation of tert-butyl methyl(2-(4-oxopiperidin-1-yl)ethyl)carbamate

A solution of DMSO (6.0 ml, 84 mmol) in dichloromethane (20 ml) was added to a solution of oxalyl chloride (2 M in DCM, 21 ml, 42 mmol) in dichloromethane (160 ml) at −78° C. The solution was stirred for 30 minutes and tert-butyl 2-(4-hydroxypiperidin-1-yl)ethyl(methyl)carbamate in 20 ml of dichloromethane was added. Stirring continued for 15 minutes and triethylamine (26.7 ml, 192 mmol) was then added. After another 15 minutes stirring, the mixture was warmed to room temperature and 50 ml of water was added. The layers were separated and the aqueous layer was extracted 3 times with dichloromethane, the combined organic layers were dried over magnesium sulfate and concentrated in vacuo. The crude was purified by flash column chromatography, eluting with 2% 2N ammonia/methanol/dichloromethane to give 6.6 g of product as a colorless oil (52% yield over two steps).

¹H NMR (400 MHz, CHLOROFORM-D) δ ppm 3.29 (br. s, 2H), 2.81 (s, 3H), 2.71 (t, J=5.82 Hz, 4H), 2.52 (t, J=6.70 Hz, 2H), 2.35 (t, J=5.60 Hz, 4H), 1.37 (s, 9H)

Part C: Preparation of 1-(2-(tert-butoxycarbonyl(methyl)amino)ethyl)-1,2,3,6-tetrahydropyridin-4-yl trifluoromethanesulfonate

To a solution of tert-butyl methyl(2-(4-oxopiperidin-1-yl)ethyl)carbamate (5.5 g, 21 mmol) in THF (60 ml) at −78° C., was added lithium bis(trimethylsilyl)amide (1 M in THF, 23 ml, 23 mmol). After stirring for 1 h, a solution of N-phenyltrifluoromethane sulfonamide (8.4 g, 23 mmol) in THF (20 ml) was added. The reaction mixture was stirred for 0.5 h at −78° C. and allowed to warm to room temperature over 3 h and quenched with saturated sodium bicarbonate. The reaction mixture was diluted with ethyl acetate and washed with 15% potassium hydrogen sulfate, saturated sodium bicarbonate solution, 1 N sodium hydroxide, water and brine. Flash chromatography (15-50% ethyl acetate/hexanes) gave the desired product as a colorless oil (7.13 g, 90% yield).

¹H NMR (400 MHz, CHLOROFORM-D) δ ppm 5.66-5.72 (m, 1H), 3.32 (s, 2H), 3.15 (s, 2H), 2.83 (s, 3H), 2.73 (br. s, 2H), 2.57 (br. s, 2H), 2.40 ((s, 2H), 1.42 (s, 9H)

Part D: Preparation of tert-butyl 2-(4-(3-fluoro-2-(2-methoxyphenyl)-1H-indol-5-yl)-5,6-dihydropyridin-1(2H)-yl)ethyl(methyl)carbamate

A mixture of 3-fluoro-2-(2-methoxyphenyl)-5-(4,4,5,5-tetramethyl-1,3,2-dioxaborolan-2-yl)-1H-indole (prepared using procedure provided for Part B of example 99) (800 mg, 2.2 mmol), 1-(2-(tert-butoxycarbonyl(methyl)amino)ethyl)-1,2,3,6-tetrahydropyridin-4-yl trifluoromethanesulfonate (846 mg, 2.2 mmol), [1,1′-bis(diphenylphosphino)ferrocene]dichloropalladium (89 mg, 0.11 mmol) and Cesium carbonate (2.1 g, 6.5 mmol) in DMF (20 ml) was heated under N₂ at 100° C. overnight. After cooling to rt, the reaction mixture was partitioned between ethyl acetate and water, The organic layer was washed with saturated sodium bicarbonate (3 times), dried over Magnesium sulfate and concentrated, purification by flash column chromatography, eluting with 1-5% 2N ammonia/methanol/dichloromethane, afforded the desired product (0.83 g, 79% yield).

MS (ESI) m/z 480 (M+H)

Part E: Preparation of 2-(4-(3-fluoro-2-(2-methoxyphenyl)-1H-indol-5-yl)piperidin-1-yl)-N-methylethanamine

Tert-butyl 2-(4-(3-fluoro-2-(2-methoxyphenyl)-1H-indol-5-yl)-5,6-dihydropyridin-1(2H)-yl)ethyl(methyl)carbamate (830 mg, 1.7 mmol) and 10% palladium on carbon (180 mg) in methanol (50 ml) was hydrogenated at 30 psi at room temperature overnight (product and starting material have the same retention time in LC/MS, but mass spectra can be used to follow the reaction). The reaction mixture was filtered through a pad of Celite and the filtrate was concentrated in vacuo to give a oil which was treated with 25% TFA in dichloroethane (10 ml) at room temperature for 1 h, solvent was evaporated and the residue was purified by preparative HPLC to afford 2-(4-(3-fluoro-2-(2-methoxyphenyl)-1H-indol-5-yl)piperidin-1-yl)-N-methylethanamine (490 mg, 47% yield).

MS (ESI) m/z 382 (M+H) ¹H NMR (CD₃OD) δ=7.80 (dd, J=9.4, 1.7 Hz, 1H), 7.29-7.42 (m, 3H), 7.02-7.14 (m, 3H), 3.96 (s, 3H), 3.70-3.78 (m, 2H), 3.56 (s, 4H), 3.19-3.30 (m, 2H), 2.95-3.04 (m, 1H), 2.82 (s, 3H), 2.10-2.22 (m, 4H).

### Example 100

### 2-(4-(3-chloro-2-(2-methoxyphenyl)-1H-indol-5-yl)piperidin-1-yl)-N-methylethanamine

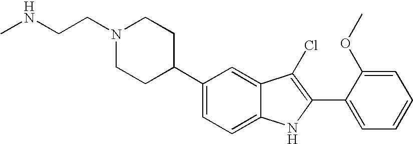

To a solution of tert-butyl 2-(4-(2-(2-methoxyphenyl)-1H-indol-5-yl)piperidin-1-yl)ethyl(methyl)carbamate (preparation as given in Part D of example 98) (43 mg, 0.09 mmol) in methanol (2 mL) was added N-chlorosuccinimide (12 mg, 0.09 mmol). The reaction mixture was stirred at room temperature for 3 hour. Solvent was evaporated and the crude intermediate was treated with 25% trifluroacetic acid in dichloroethane (2 mL) at room temperature for 1 h. After removal of solvent in vacuo, The residue was purified using preparative HPLC using the conditions given below to afford 37 mg of 2-(4-(3-chloro-2-(2-methoxyphenyl)-1H-indol-5-yl)piperidin-1-yl)-N-methylethanamine (Example 100).

Conditions: Column—YMC ODS (20×50 mm)

Solvents—A-90% water-10% methanol-0.1% TFA

B-10% water-90% methanol-0.1% TFA

Gradient-25% B to 100% B in 12 min

Retention time: 8.3 min

MS (ESI) m/z 398 (M+H) ¹H NMR (CD₃OD) δ=7.69 (dd, J=7.5, 1.8 Hz, 1H), 7.35-7.42 (m, 3H), 7.22 (t, J=8.4 Hz, 2H), 7.11 (dt, J=7.5, 0.9 Hz, 1H), 3.87 (s, 3H), 3.72-3.79 (m, 2H), 3.56 (s, 4H), 3.18-3.30 (m, 2H), 2.97-3.06 (m, 1H), 2.82 (s, 3H), 2.14-2.21 (m, 4H).

### Example 101

### 2-(2-methoxyphenyl)-5-(1-(2-(methylamino)ethyl)piperidin-4-yl)-1H-indole-3-carbonitrile

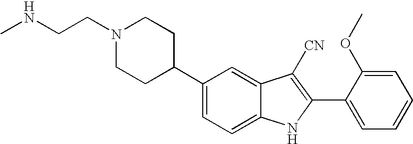

To a solution of tert-butyl 2-(4-(2-(2-methoxyphenyl)-1H-indol-5-yl)piperidin-1-yl)ethyl(methyl)carbamate (preparation as given in Part D of example 98) (100 mg, 0.22 mmol) in acetonitrile (2 mL) was added chlorosulfonyl isocyanate (19 μL, 0.22 mmol) at 0° C. The reaction mixture was stirred at 0° C. for half an hour and anhydrous dimethylformamide (18 μL, 0.24 mmol) was added. The mixture was stirred at room temperature for 1 hour and 1N sodium hydroxide (1 mL) was added and subsequently the mixture was heated at 80° C. for 5 minutes. The reaction mixture was cooled to room temperature and was partitioned between ethyl acetate and water. The organic layer was washed with saturated sodium bicarbonate and water, dried over MgSO₄ and concentrated, The crude intermediate was treated with 25% trifluroacetic acid in dichloroethane (3 mL) at room temperature for 1 h. Solvent was evaporated and the residue was purified by preparative HPLC using conditions given below to afford 43 mg of 2-(2-methoxyphenyl)-5-(1-(2-(methylamino)ethyl)piperidin-4-yl)-1H-indole-3-carbonitrile (Example 101)

Conditions: Column—YMC ODS (20×50 mm)

Solvents—A-90% water-10% methanol-0.1% TFA

B-10% water-90% methanol-0.1% TFA

Gradient-25% B to 100% B in 12 min

Retention time: 8.3 min

MS (ESI) m/z 389 (M+H) ¹H NMR (CD₃OD) δ=7.79 (dd, J=7.7, 1.6 Hz, 1H), 7.46-7.54 (m, 3H), 7.22 (t, J=8.8 Hz, 2H), 7.11 (dt, J=7.5, 1.1 Hz, 1H), 3.95 (s, 3H), 3.72-3.80 (m, 2H), 3.56 (s, 4H), 3.22-3.30 (m, 2H), 3.00-3.10 (m, 1H), 2.82 (s, 3H), 2.15-2.22 (m, 4H).

### Example 102

### 2-(4-(2-(2-methoxyphenyl)-3-methyl-1H-indol-5-yl)piperidin-1-yl)-N-methylethanamine

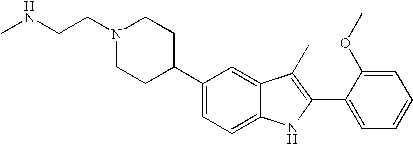 Part A: Preparation of 3-methyl-5-(4,4,5,5-tetramethyl-1,3,2-dioxaborolan-2-yl)-1H-indole

A mixture of 3-methyl-5-bromo-indole (0.86 g, 4.1 mmol), bis(pinacolato)diboron (1.73 g, 6.1 mmol), [1,1′-bis(diphenylphosphino)ferrocene]dichloropalladium (0.17 g, 0.20 mmol) and potassium acetate (1.20 g, 12.3 mmol) in methyl sulfoxide (6 mL) was heated at 80° C. under N₂ overnight, the reaction mixture was partitioned between EtOAc and water. Solid was filtered and the organic layer was washed 3 times with saturated sodium bicarbonate, dried over MgSO₄ and concentrated, The crude product was purified by flash column chromatography, eluting with 10% EA/Hexane to give a white solid (0.84 g, 80% yield).

Part B: Preparation of tert-butyl 4-(3-methyl-1H-indol-5-yl)-5,6-dihydropyridine-1(2H)-carboxylate

A mixture of 3 3-methyl-5-(4,4,5,5-tetramethyl-1,3,2-dioxaborolan-2-yl)-1H-indole (0.84 g, 3.3 mmol), tert-butyl 4-(trifluoromethylsulfonyloxy)-5,6-dihydropyridine-1(2H)-carboxylate (1.30 g, 3.9-mmol), [1,1′-bis(diphenylphosphino)ferrocene]dichloropalladium (0.16 g, 0.20 mmol) and Cesium carbonate (3.21 g, 9.8 mmol) in DMF (12 mL) was heated at 100° C. under N₂ overnight. The reaction mixture was partitioned between EtOAc and water, The organic layer was washed with saturated sodium bicarbonate (3×), dried over MgSO₄ and concentrated, purification by flash column chromatography, eluting with 10% EA/Hexane, afforded the desired product as an colorless oil (0.76 g, 74% yield).

Part C: Preparation of tert-butyl 4-(3-methyl-1H-indol-5-yl)piperidine-1-carboxylate

Tert-butyl 4-(3-methyl-1H-indol-5-yl)-5,6-dihydropyridine-1(2H)-carboxylate (0.19 g, 0.62 mmol) and 10% palladium on carbon (65 mg) in methanol was hydrogenated at 35 psi at room temperature for 3 hours. The reaction mixture was filtered through a pad of Celite and the filtrate was concentrated in vacuo to give tert-butyl 4-(3-methyl-1H-indol-5-yl)piperidine-1-carboxylate (0.12 g, 63%) as a colorless oil.

Part D: Preparation of 2-(4-(2-(2-methoxyphenyl)-3-methyl-1H-indol-5-yl)piperidin-1-yl)-N-methylethanamine

Pyridine tribromide (0.13 g, 0.58 mmol) was added in one batch to a solution of tert-butyl 4-(3-methyl-1H-indol-5-yl)piperidine-1-carboxylate (0.12 g, 0.39 mmol) in a 1:1 mixture of THF and CHCl3 (4 mL) at 0° C. The cooling bath was removed and the reaction mixture was stirred at rt for 2 hours. The reaction mixture was diluted with ethyl acetate and washed with NaHSO₃, NaHCO₃ and water. The organic layer was dried over MgSO₄, concentrated and purified by flash column chromatography, eluting with 10% EA/Hexane.

A mixture of tert-butyl 4-(2-bromo-3-methyl-1H-indol-5-yl)piperidine-1-carboxylate obtained above, 2-methoxyphenylboronic acid, palladium tetrakistriphenylphosphine and cesium carbonate in DMF was heated at 90° C. overnight. The reaction mixture was diluted with ethyl acetate and washed with NaHCO₃ and water. The organic layer was dried over MgSO₄, concentrated and the crude tert-butyl 4-(2-(2-methoxyphenyl)-3-methyl-1H-indol-5-yl)piperidine-1-carboxylate was used without purification (90 mg, 56% yield).

Tert-butyl 4-(2-(2-methoxyphenyl)-3-methyl-1H-indol-5-yl) piperidine-1-carboxylate was treated with 25% TFA in dichloroethane (3 mL) at rt for 1 h. 2M ammonia in methanol was added to neutralize the acid. Solvent was evaporated and the residue was extracted with dichloromethane. Solvent was evaporated and the crude was dissolved in dichloroethane, tert-butyl methyl(2-oxoethyl)carbamate (50 mg, 0.29 mmol), sodium triacetoxyboronhydride (86 mg, 0.40 mmol), and acetic acid (17 mg, 0.29 mmol) were added and the reaction mixture was stirred at rt for 4 h. after aqueous workup, the organic layer was dried and concentrated. The residue was treated with 25% TFA in dichloroethane (3 mL) at rt for 1 h, diluted with dichloromethane and washed with saturated sodium bicarbonate, dried over magnesium sulfate and concentrated. Purification by preparative HPLC using conditions given below afforded 2-(4-(2-(2-methoxyphenyl)-3-methyl-1H-indol-5-yl)piperidin-1-yl)-N-methylethanamine (Example 100) (18 mg, 12% yield).

Conditions: Column—YMC ODS (20×50 mm)

Solvents—A-90% water-10% methanol-0.1% TFA

B-10% water-90% methanol-0.1% TFA

Gradient-25% B to 100% B in 12 min

Retention time: 10 min

MS (ESI) m/z 378 (M+H) ¹H NMR (CD₃OD) δ=7.34-7.41 (m, 3H), 7.30 (d, J=8.3 Hz, 1H), 7.10 (d, J=8.3 Hz, 1H), 7.03 (q, J=7.7 Hz, 2H), 3.83 (s, 3H), 3.72-3.78 (m, 2H), 3.56 (s, 4H), 3.25 (br s, 2H), 2.95-3.03 (m, 1H), 2.82 (s, 3H), 2.26 (s, 3H), 2.15-2.20 (m, 4H).

### Examples 103 to 132

Examples 103 through 132 as shown in Table 2 were prepared using similar procedure as reported for example 102.

TABLE 2

| Example # | Structure | MS (m/z) |
|---|---|---|
| 103 | 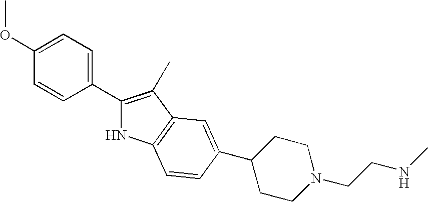 | 378.5 |
| 104 | 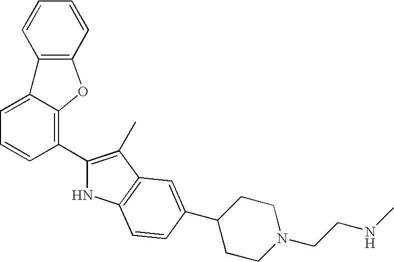 | 438.6 |
| 105 | 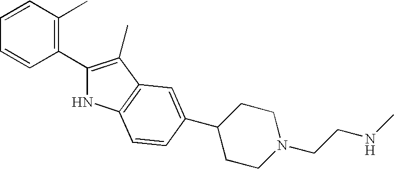 | 362.5 |
| 106 | 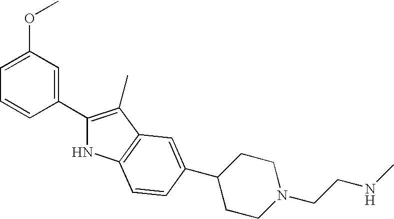 | 378.5 |
| 107 | 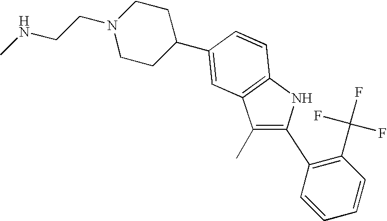 | 416.5 |
| 108 | 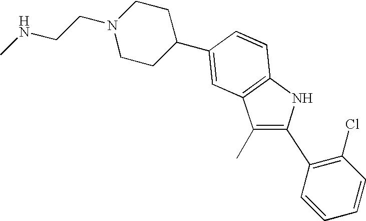 | 383 |
| 109 | 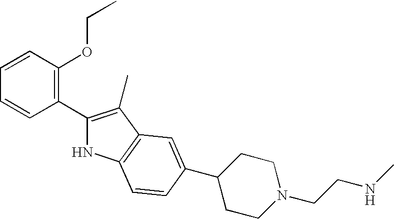 | 392.6 |
| 110 | 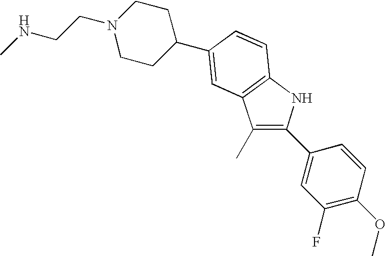 | 396.5 |
| 111 | 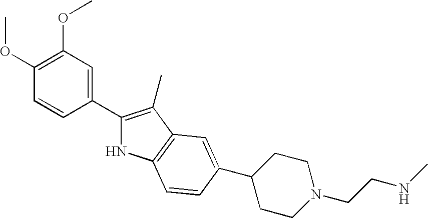 | 408.6 |
| 112 | 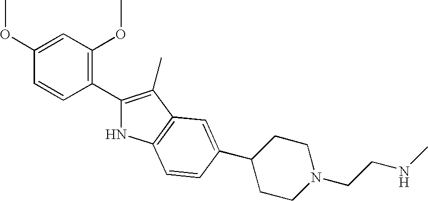 | 408.6 |
| 113 | 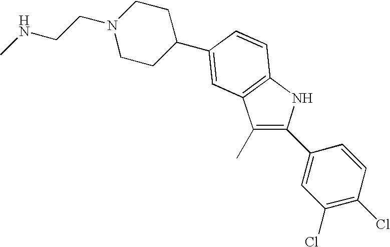 | 417.4 |
| 114 | 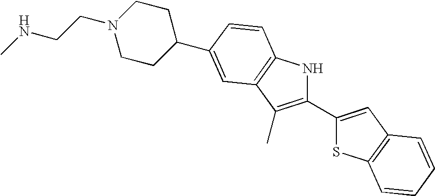 | 404.6 |
| 115 | 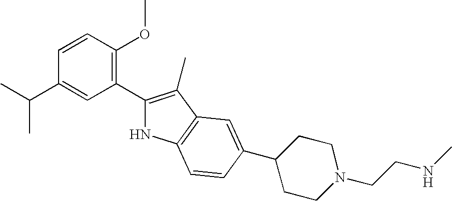 | 420.6 |
| 116 | 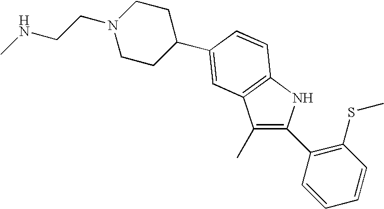 | 394.6 |
| 117 | 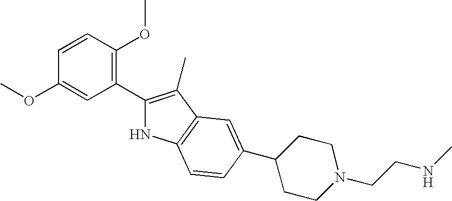 | 408.6 |
| 118 | 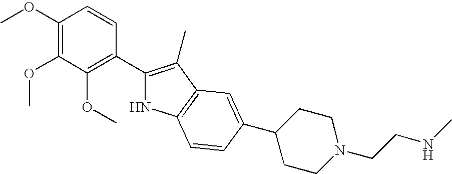 | 438.6 |
| 119 | 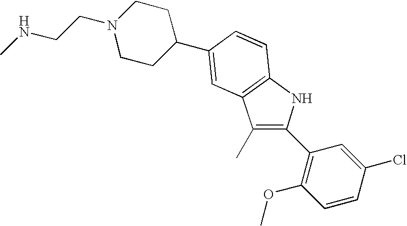 | 413 |
| 120 | 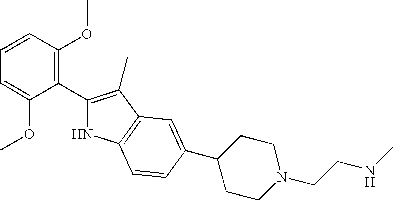 | 408.6 |
| 121 | 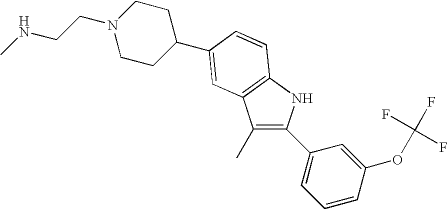 | 432.5 |
| 122 | 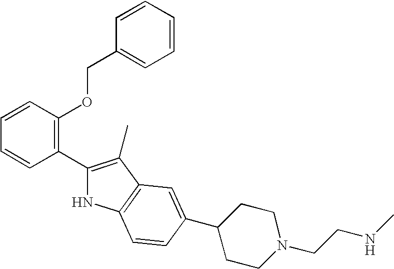 | 454.6 |
| 123 | 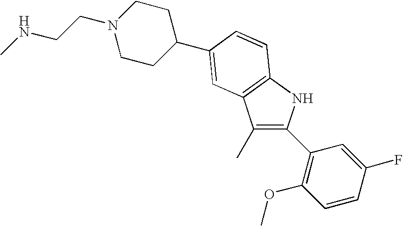 | 396.5 |
| 124 | 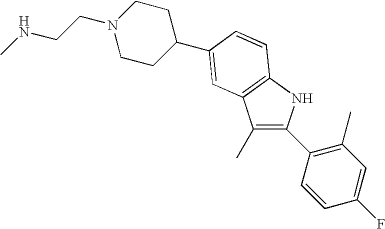 | 380.5 |
| 125 | 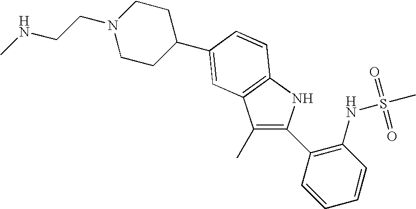 | 441.6 |
| 126 | 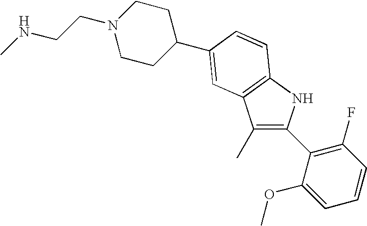 | 396.5 |
| 127 | 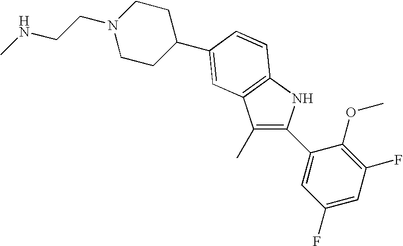 | 414.5 |
| 128 | 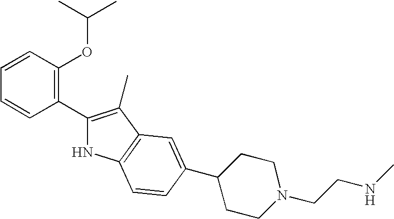 | 406.6 |
| 129 | 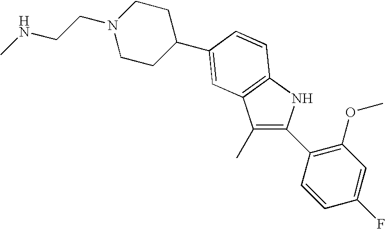 | 396.5 |
| 130 | 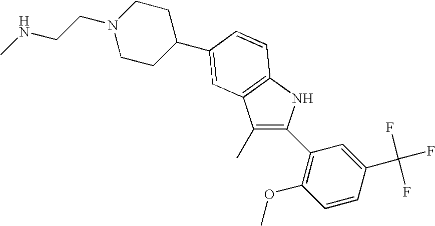 | 446.5 |
| 131 | 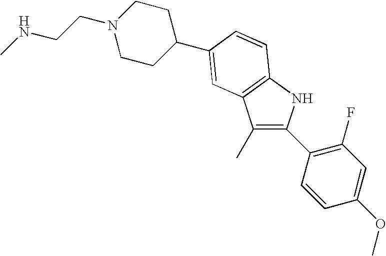 | 396.5 |
| 132 | 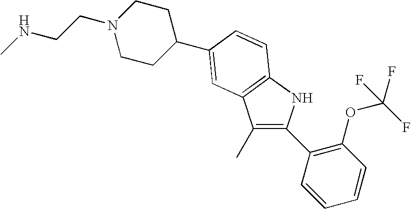 | 432.51 |

### Example 133

### 2-(4-(2-(2-methoxyphenyl)-1H-indol-5-yl)azepan-1-yl)-N-methylethanamnine

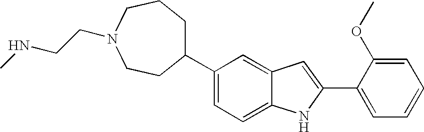 Part A: Preparation of (E)-tert-butyl 4-(trifluoromethylsulfonyloxy)-2,3,6,7-tetrahydro-1H-azepine-1-carboxylate

To a solution of tert-butyl 4-oxoazepane-1-carboxylate (213 mg, 1.0 mmol) in THF (15 ml) at −78° C. was added lithium bis(trimethylsilyl)amide (1 M in THF, 1.1 ml, 1.1 mmol) and the reaction solution was stirred for 1 h. A solution of N-(5-chloropyridin-2-yl)-1,1,1-trifluoro-N-(trifluoromethylsulfonyl)methanesulfonamide (432 mg, 1.1 mmol) in THF (10 ml) was added drop wise. The reaction mixture was stirred for another 0.5 h and allowed to warm to room temperature over 3 h and quenched with saturated sodium bicarbonate. The reaction mixture was diluted with ethyl acetate and washed with 15% potassium hydrogen sulfate, saturated sodium bicarbonate solution, 1 N sodium hydroxide, water and brine. The combined organic layers were dried over magnesium sulfate and concentrated in vacuo. Flash chromatography (silica gel, 10% ethyl acetate/hexane) gave the product as a colorless oil (200 mg, 58% yield)

Part B: Preparation of (E)-tert-butyl 4-(2-(2-methoxyphenyl)-1H-indol-5-yl)-2,3,6,7-tetrahydro-1H-azepine-1-carboxylate

A mixture of 2-(2-methoxyphenyl)-5-(4,4,5,5-tetramethyl-1,3,2-dioxaborolan-2-yl)-1H-indole (prepared using procedure provided for Part C of example 98) (202 mg, 0.58 mmol), (E)-tert-butyl 4-(trifluoromethylsulfonyloxy)-2,3,6,7-tetrahydro-1H-azepine-1-carboxylate (200 mg, 0.58 mmol), [1,1′-bis(diphenylphosphino)ferrocene]dichloropalladium (28 mg, 0.035 mmol) and Cesium carbonate (567 mg, 1.74 mmol) in DMF (5 ml) was heated at 100° C. under nitrogen overnight. The reaction mixture was partitioned between ethyl acetate and water, The organic layer was washed with saturated sodium bicarbonate (3 times), dried over magnesium sulfate and concentrated in vacuo, purification by flash column chromatography, eluting with 10% ethyl acetate/hexane, afforded the desired product as a brown oil (193 mg, 80% yield).

MS (ESI) m/z 419 (M+H). ¹H NMR (400 MHz, CHLOROFORM-D) δ ppm 9.66 (br. s, 1H), 7.83 (dd, J=7.69, 1.76 Hz, 1H), 7.51-7.59 (m, 1H), 7.25-7.35 (m, 2H), 7.16 (td, J=8.24, 1.76 Hz, 1H), 7.00-7.09 (m, 2H), 6.88 (br. s, 1H), 5.99-6.05 (m, 1H), 3.98-4.08 (m, 4H), 3.62-3.69 (m, 2H), 3.54-3.62 (m, 1H), 2.76-2.87 (m, 2H), 2.46 (q, J=5.79 Hz, 1H), 1.89-1.97 (m, 1H), 1.50 (d, J=7.69 Hz, 9H).

Part C: (E)-tert-butyl 2-(4-(2-(2-methoxyphenyl)-1H-indol-5-yl)-2,3,6,7-tetrahydro-1H-azepin-1-yl)ethyl(methyl)carbamate

(E)-tert-butyl 4-(2-(2-methoxyphenyl)-1H-indol-5-yl)-2,3,6,7-tetrahydro-1H-azepine-1-carboxylate (193 mg, 0.46 mmol) was treated with 3 ml of 25% TFA in dichloroethane at room temperature for 1 h. Solvent was evaporated and the residue was purified by flush column chromatography, eluting with 3% 2N ammonia/methanol/dichloromethane to give the intermediate as a brown oil.

A mixture of the above intermediate, tert-butyl methyl(2-oxoethyl)carbamate (preparation as given in Part A of example 1) (80 mg, 0.46 mmol), sodium triacetoxyboronhydride (137 mg, 0.65 mmol), and acetic acid (28 mg, 0.46 mmol) in dichloroethane (10 ml) was stirred at room temperature overnight. The reaction mixture was diluted with ethyl acetate and washed 3 times with saturated sodium bicarbonate. The organic layer was dried over magnesium sulfate and concentrated in vacuo, the crude was used without purification (190 mg).

MS (ESI) m/z 476 (M+H).

Part D: 2-(4-(2-(2-methoxyphenyl)-1H-indol-5-yl)azepan-1-yl)-N-methylethanamine

A mixture of (E)-tert-butyl 2-(4-(2-(2-methoxyphenyl)-1H-indol-5-yl)-2,3,6,7-tetrahydro-1H-azepin-1-yl)ethyl(methyl)carbamate (190 mg, 0.40 mmol) and 10% palladium on carbon (42 mg) in methanol (30 ml) was hydrogenated at 30 psi overnight. The reaction mixture was filtered through a pad of Celite and the filtrate was concentrated in vacuo to give an oil which was subsequently treated with 25% TFA in dichloroethane (3 ml) room temperature for 1 h. Solvent was evaporated and the residue was purified by preparative HPLC to afford the desired product as a brown oil (97 mg, 40% yield).

MS (ESI) m/z 378 (M+H). ¹H NMR (400 MHz, METHANOL-D3) δ ppm 7.76 (dd, J=7.9, 1.8 Hz, 1H), 7.39 (d, J=1.3 Hz, 1H), 7.37 (d, J=8.8 Hz, 1H), 7.25-7.30 (m, 1H), 7.10 (d, J=8.4 Hz, 1H), 6.98-7.04 (m, 2H), 3.97 (s, 3H), 3.50-3.61 (m, 8H), 2.92-2.99 (m, 1H), 2.80 (s, 3H), 1.85-2.35 (m, 6H).

### Example 134

### 2-(10-(2-(2-methoxyphenyl)-1H-indol-5-yl)-7-azaspiro[4.5]decan-7-yl)-N-methylethanamine

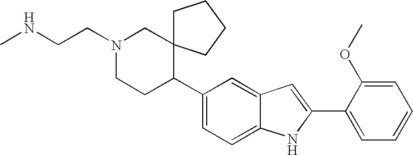 Part A: Tert-butyl 10-(trifluoromethylsulfonyloxy)-7-azaspiro [4.5]dec-9-ene-7-carboxylate

To a solution of tert-butyl 10-oxo-7-azaspiro[4.5]decane-7-carboxylate (506 mg, 2.0 mmol) in THF (15 ml) at −78° C. was added lithium bis(trimethylsilyl)amide (1 M in THF, 2.2 ml, 2.2 mmol) and the reaction solution was stirred for 1 h. A solution of N-(5-chloropyridin-2-yl)-1,1,1-trifluoro-N-(trifluoromethylsulfonyl)methanesulfonamide (864 mg, 2.2 mmol) in THF (20 ml) was added drop wise. The reaction mixture was stirred for another 0.5 h and allowed to warm to room temperature over 3 h and quenched with saturated sodium bicarbonate. The reaction mixture was diluted with ethyl acetate and washed with 15% potassium hydrogen sulfate, saturated sodium bicarbonate solution, 1 N sodium hydroxide, water and brine. The combined organic layers were dried over magnesium sulfate and concentrated in vacuo. Flash chromatography (silica gel, 10% ethyl acetate/hexane) gave the product as a colorless oil (433 mg, 56% yield).

¹H NMR (400 MHz, CHLOROFORM-D) δ ppm 5.60-5.727 (m, 1H), 4.04 (d, J=10.11 Hz, 2H), 3.36 (s, 2H), 1.65-1.77 (m, 7H), 1.51-1.56 (m, 1H), 1.45 (s, 9H).

Part B: Tert-butyl 10-(2-(2-methoxyphenyl)-1H-indol-5-yl)-7-azaspiro[4.5]dec-9-ene-7-carboxylate

A mixture of 2-(2-methoxyphenyl)-5-(4,4,5,5-tetramethyl-1,3,2-dioxaborolan-2-yl)-1H-indole (prepared using procedure provided for Part C of example 98) (192 mg, 0.55 mmol), tert-butyl 10-(trifluoromethylsulfonyloxy)-7-azaspiro[4.5]dec-9-ene-7-carboxylate (212 mg, 0.55 mmol), [1,1′-bis(diphenylphosphino)ferrocene]dichloropalladium (27 mg, 0.033 mmol) and Cesium carbonate (538 mg, 1.70 mmol) in DMF (3 ml) was heated at 100° C. under nitrogen overnight. The reaction mixture was partitioned between ethyl acetate and water, The organic layer was washed with saturated sodium bicarbonate (3 times), dried over magnesium sulfate and concentrated in vacuo, purification by flash column chromatography, eluting with 10% ethyl acetate/hexane, afforded the desired product as a brown oil (200 mg, 80% yield).

MS (ESI) m/z 459 (M+H). ¹H NMR (400 MHz, CHLOROFORM-D) δ ppm 7.83 (dd, J=7.69, 1.76 Hz, 1H), 7.38 (s, 1H), 7.24-7.34 (m, 2H), 7.01-7.08 (m, 2H), 6.97 (dd, J=8.35, 1.32 Hz, 1H), 5.54 (br. s, 1H), 4.02 (s, 2H), 4.00 (s, 3H), 3.37 (s, 2H), 1.62-1.82 (m, 5H), 1.48-1.58 (m, 12H).

Part C: Tert-butyl 2-(10-(2-(2-methoxyphenyl)-1H-indol-5-yl)-7-azaspiro[4.5]dec-9-en-7-yl)ethyl(methyl)carbamate

Tert-butyl 10-(2-(2-methoxyphenyl)-1H-indol-5-yl)-7-azaspiro[4.5]dec-9-ene-7-carboxylate (200 mg, 0.44 mmol) was treated with 3 ml of 25% TFA in dichloroethane at room temperature for 1 h. Solvent was evaporated and the residue was purified by flush column chromatography, eluting with 3% 2N ammonia/methanol/dichloromethane to give the intermediate as a brown oil.

A mixture of the above intermediate, tert-butyl methyl(2-oxoethyl)carbamate (preparation as given in Part A of example 1) (76 mg, 0.44 mmol), sodium triacetoxyboronhydride (130 mg, 0.61 mmol), and acetic acid (26 mg, 0.44 mmol) in dichloroethane (10 ml) was stirred at room temperature overnight. The reaction mixture was diluted with ethyl acetate and washed 3 times with saturated sodium bicarbonate. The organic layer was dried over magnesium sulfate and concentrated in vacuo, the crude was used without purification (221 mg).

MS (ESI) m/z 516 (M+H).

Part D: 2-(10-(2-(2-methoxyphenyl)-1H-indol-5-yl)-7-azaspiro[4.5]decan-7-yl)-N-methylethanamine

A mixture of crude tert-butyl 2-(10-(2-(2-methoxyphenyl)-1H-indol-5-yl)-7-azaspiro[4.5]dec-9-en-7-yl)ethyl(methyl)carbamate (221 mg, 0.43 mmol) and 10% palladium on carbon (46 mg) in methanol (30 ml) was hydrogenated at 30 psi overnight. The reaction mixture was filtered through a pad of Celite and the filtrate was concentrated in vacuo to give an oil which was subsequently treated with 25% TFA in dichloroethane (3 ml) room temperature for 1 h. Solvent was evaporated and the residue was purified by preparative HPLC to afford the desired product as a brown oil (200 mg, 72% yield).

MS (ESI) m/z 418 (M+H). ¹H NMR (400 MHz, METHANOL-D3) δ ppm 7.77 (dd, J=7.80, 1.65 Hz, 1H), 7.39 (d, J=8.79 Hz, 2H), 7.28 (ddd, J=8.51, 7.20, 1.65 Hz, 1H), 7.10 (d, J=7.47 Hz, 1H), 6.97-7.04 (m, 2H), 3.97 (s, 3H), 3.74 (s, 1H), 3.51-3.63 (m, 5H), 3.07-3.15 (m, 2H), 2.93 (dd, J=12.96, 2.86 Hz, 1H), 2.81 (s, 3H), 2.46-2.59 (m, 1H), 1.91-2.02 (m, 3H), 1.67-1.75 (m, 1H), 1.47-1.59 (m, 1H), 1.28-1.42 (m, 2H), 1.14-1.28 (m, 1H), 0.86-1.00 (m, 1H).

### Example 135

### 2-(3-(2-(2-methoxyphenyl)-1H-indol-5-yl)-8-azabicyclo[3.2.1]octan-8-yl)-N-methylethanamine

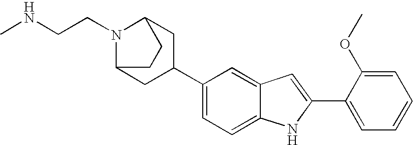 Part A: Tert-butyl 3-(trifluoromethylsulfonyloxy)-8-azabicyclo[3.2.1]oct-3-ene-8-carboxylate

To a solution of tert-butyl 3-oxo-8-azabicyclo[3.2.1 ]octane-8-carboxylate (450 mg, 2.0 mmol) in THF (15 ml) at −78° C. was added lithium bis(trimethylsilyl)amide (1 M in THF, 2.2 ml, 2.2 mmol) and the reaction solution was stirred for 1 h. A solution of N-(5-chloropyridin-2-yl)-1,1,1-trifluoro-N-(trifluoromethylsulfonyl)methanesulfonamide (864 mg, 2.2 mmol) in THF (20 ml) was added drop wise. The reaction mixture was stirred for another 0.5 h and allowed to warm to room temperature over 3 h and quenched with saturated sodium bicarbonate. The reaction mixture was diluted with ethyl acetate and washed with 15% potassium hydrogen sulfate, saturated sodium bicarbonate solution, 1 N sodium hydroxide, water and brine. The combined organic layers were dried over magnesium sulfate and concentrated in vacuo. Flash chromatography (silica gel, 10% ethyl acetate/hexane) gave the product as a colorless oil (659 mg, 92% yield)

¹H NMR (400 MHz, CHLOROFORM-D) δ ppm 6.08 (s, 1H), 4.32-4.58 (m, 2H), 2.90-3.15 (m, 1H), 2.23 (br. s, 1H), 1.98-2.07 (m, 3H), 1.67-1.78 (m, 1H), 1.57 (s, 1H), 1.45 (s, 9H).

Part B: Tert-butyl 3-(2-(2-methoxyphenyl)-1H-indol-5-yl)-8-azabicyclo[3.2.1]oct-3-ene-8-carboxylate

A mixture of 2-(2-methoxyphenyl)-5-(4,4,5,5-tetramethyl-1,3,2-dioxaborolan-2-yl)-1H-indole (prepared using procedure provided for Part C of example 98) (279 mg, 0.80 mmol), tert-butyl 3-(trifluoromethylsulfonyloxy)-8-azabicyclo[3.2.1]oct-3-ene-8-carboxylate (286 mg, 0.80 mmol), [1,1′-bis(diphenylphosphino)ferrocene]dichloropalladium (39 mg, 0.048 mmol) and Cesium carbonate (782 mg, 2.40 mmol) in DMF (4 ml) was heated at 100° C. under nitrogen overnight. The reaction mixture was partitioned between ethyl acetate and water, The organic layer was washed with saturated sodium bicarbonate (3 times), dried over magnesium sulfate and concentrated in vacuo, purification by flash column chromatography, eluting with 10% ethyl acetate/hexane, afforded the desired product as white solid (287 mg, 83% yield).

MS (ESI) m/z 431 (M+H). ¹H NMR (400 MHz, CHLOROFORM-D) δ ppm 9.72 (br. s, 1H), 7.84 (dd, J=7.80, 1.65 Hz, 1H), 7.58 (d, J=1.54 Hz, 1H), 7.32-7.36 (m, 1H), 7.23-7.31 (m, 3H), 7.01-7.08 (m, 2H), 6.37-6.43 (m, 1H), 4.46-4.54 (m, 2H), 4.02 (s, 3H), 3.19 (dd, J=17.03, 4.28 Hz, 1H), 2.34 (d, J=16.70 Hz, 1H), 2.16-2.26 (m, 1H), 1.93-2.04 (m, 2H), 1.69-1.78 (m, 1H), 1.40-1.48 (m, 9H).

Part C: Tert-butyl 2-(3-(2-(2-methoxyphenyl)-1H-indol-5-yl)-8-azabicyclo[3.2.1]oct-3-en-8-yl)ethyl(methyl)carbamate

Tert-butyl 3-(2-(2-methoxyphenyl)-1H-indol-5-yl)-8-azabicyclo[3.2.1]oct-3-ene-8-carboxylate (287 mg, 0.67 mmol) was treated with 3 ml of 25% TFA in dichloroethane at room temperature for 1 h. Solvent was evaporated and the residue was purified by flush column chromatography, eluting with 5% 2N ammonia/methanol/dichloromethane to give the intermediate.

A mixture of the above intermediate, tert-butyl methyl(2-oxoethyl)carbamate (preparation as given in Part A of example 1) (115 mg, 0.67 mmol), sodium triacetoxyboronhydride (198 mg, 0.93 mmol), and acetic acid (40 mg, 0.67 mmol) in dichloroethane (10 ml) was stirred at room temperature overnight. The reaction mixture was diluted with ethyl acetate and washed 3 times with saturated sodium bicarbonate. The organic layer was dried over magnesium sulfate and concentrated in vacuo, the crude was purified by flash column chromatography, eluting with 3% 2N ammonia/methanol/dichloromethane to give the desired product (262 mg, 80 yield).

MS (ESI) m/z 488 (M+H). ¹H NMR (400 MHz, CHLOROFORM-D) δ ppm 9.67 (s, 1H), 7.83 (dd, J=7.80, 1.65 Hz, 1H), 7.60 (s, 1H), 7.25-7.35 (m, 3H), 6.99-7.08 (m, 2H), 6.87 (d, J=1.32 Hz, 1H), 6.25 (d, J=5.27 Hz, 1H), 5.28 (s, 1H), 4.00 (s, 3H), 3.34-3.62 (m, 4H), 2.90-3.09 (m, 1H), 2.88 (s, 3H), 2.73 (t, J=7.36 Hz, 2H), 2.12-2.23 (m, 2H), 2.00-2.10 (m, 1H), 1.88-1.96 (m, 1H), 1.59-1.69 (m, 1H), 1.40-1.48 (m, 10H).

Part D: 2-(3-(2-(2-methoxyphenyl)-1H-indol-5-yl)-8-azabicyclo[3.2.1]octan-8-yl)-N-methylethanamine

A mixture of tert-butyl 2-(3-(2-(2-methoxyphenyl)-1H-indol-5-yl)-8-azabicyclo[3.2.1]oct-3-en-8-yl)ethyl(methyl)carbamate (262 mg, 0.54 mmol) and 10% palladium on carbon (60 mg) in methanol (30 ml) was hydrogenated at 30 psi overnight. The reaction mixture was filtered through a pad of Celite and the filtrate was concentrated in vacuo to give an oil which was subsequently treated with 25% TFA in dichloroethane (3 ml) room temperature for 1 h. Solvent was evaporated and the residue was purified by preparative HPLC to afford the desired product as a brown oil (361 mg, 99% yield).

MS (ESI) m/z 390 (M+H). ¹H NMR (400 MHz, METHANOL-D3) δ ppm 7.76 (dd, J=7.7, 1.5 Hz, 1H), 7.66 (s, 1H), 7.36-7.45 (m, 1H), 7.25-7.30 (m, 1H), 7.19 (dd, J=8.6, 1.5 Hz, 1H), 6.98-7.12 (m, 2H), 4.08 (br s, 2H), 3.97 (s, 3H), 3.32-3.58 (m, 5H), 2.79 (s, 3H), 2.61-2.73 (m, 3H), 2.22-2.39 (m, 1H), 1.94-2.09 (m, 4H).

### Example 136

### 2-(3-(2-(2-methoxyphenyl)-1H-indol-5-yl)pyrrolidin-1-yl)-N-methylethanamine

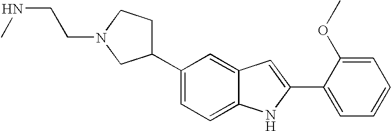 Part A: Tert-butyl 3-(trifluoromethylsulfonyloxy)-2,5-dihydro-1H-pyrrole-1-carboxylate

To a solution of tert-butyl 3-oxopyrrolidine-1-carboxylate (370 mg, 2.0 mmol) in THF (15 ml) at −78° C. was added lithium bis(trimethylsilyl)amide (1M in THF, 2.2 ml, 2.2 mmol) and the reaction solution was stirred for 1 h. A solution of N-(5-chloropyridin-2-yl)-1,1,1-trifluoro-N-(trifluoromethylsulfonyl)methanesulfonamide (864 mg, 2.2 mmol) in THF (20 ml) was added drop wise. The reaction mixture was stirred for another 0.5 h and allowed to warm to room temperature over 3 h and quenched with saturated sodium bicarbonate. The reaction mixture was diluted with ethyl acetate and washed with 15% potassium hydrogen sulfate, saturated sodium bicarbonate solution, 1 N sodium hydroxide, water and brine. The combined organic layers were dried over magnesium sulfate and concentrated in vacuo. Flash chromatography (silica gel, 10% ethyl acetate/hexane) gave the product as a colorless oil (214 mg, 34% yield)

Part B: Tert-butyl 3-(2-(2-methoxyphenyl)-1H-indol-5-yl)-2,5-dihydro-1H-pyrrole-1-carboxylate

A mixture of 2-(2-methoxyphenyl)-5-(4,4,5,5-tetramethyl-1,3,2-dioxaborolan-2-yl)-1H-indole (prepared using procedure provided for Part C of example 98) (234 mg, 0.68 mmol), tert-butyl 3-(trifluoromethylsulfonyloxy)-2,5-dihydro-1H-pyrrole-1-carboxylate (214 mg, 0.68 mmol), [1,1′-bis(diphenylphosphino)ferrocene]dichloropalladium (33 mg, 0.040 mmol) and Cesium carbonate (660 mg, 2.02 mmol) in DMF (4 ml) was heated at 100° C. under nitrogen overnight. The reaction mixture was partitioned between ethyl acetate and water, The organic layer was washed with saturated sodium bicarbonate (3 times), dried over magnesium sulfate and concentrated in vacuo, purification by flash column chromatography, eluting with 10% ethyl acetate/hexane, afforded the desired product as a brown oil (42 mg, 16% yield).

MS (ESI) m/z 391 (M+H). ¹H NMR (400 MHz, CHLOROFORM-D) δ ppm 9.72 (s, 1H), 7.83 (dd, J=7.69, 1.76 Hz, 1H), 7.56 (s, 1H), 7.36-7.39 (m, 1H), 7.26-7.33 (m, 2H), 7.01-7.10 (m, 2 H), 6.88 (br. s, 1H), 6.07 (s, 1H), 4.58 (br. s, 2H), 4.32 (br. s, 2H), 4.02 (s, 3H), 1.57-1.66 (m, 1H), 1.52 (s, 9H), 1.22-1.32 (m, 2H), 0.82-0.90 (m, 1H).

Part C: Tert-butyl 2-(3-2-2-methoxyphenyl)-1H-indol-5-yl)-2,5-dihydro-1H-pyrrol-1-yl)ethyl(methyl)carbamate

Tert-butyl 3-(2-(2-methoxyphenyl)-1H-indol-5-yl)-2,5-dihydro-1H-pyrrole-1-carboxylate (42 mg, 0.11 mmol) was treated with 2 ml of 25% TFA in dichloroethane at room temperature for 1 h. Solvent was evaporated and the residue was purified by flush column chromatography, eluting with 3% 2N ammonia/methanol/dichloromethane to give the intermediate as a brown oil.

A mixture of the above intermediate, tert-butyl methyl(2-oxoethyl)carbamate (preparation as given in Part A of example 1) (20 mg, 0.11 mmol), sodium triacetoxyboronhydride (32 mg, 0.15 mmol), and acetic acid (6.5 mg, 0.11 mmol) in dichloroethane (10 ml) was stirred at room temperature overnight. The reaction mixture was diluted with ethyl acetate and washed 3 times with saturated sodium bicarbonate. The organic layer was dried over magnesium sulfate and concentrated in vacuo, the crude was used without purification (31 mg).

MS (ESI) m/z 448 (M+H).

Part D: 2-(3-(2-(2-methoxyphenyl)-1H-indol-5-yl)pyrrolidin-1-yl)-N-methylethananiine

A mixture of crude tert-butyl 2-(3-(2-(2-methoxyphenyl)-1H-indol-5-yl)-2,5-dihydro-1H-pyrrol-1-yl)ethyl(methyl)carbamate (31 mg, 0.069 mmol) and 10% palladium on carbon (7 mg) in methanol (30 ml) was hydrogenated at 30 psi overnight. The reaction mixture was filtered through a pad of Celite and the filtrate was concentrated in vacuo to give tert-butyl 2-(3-(2-(2-methoxyphenyl)-1H-indol-5-yl)pyrrolidin-1-yl)ethyl(methyl)carbamate which was subsequently treated with 25% TFA in dichloroethane (3 ml) room temperature for 1 h. Solvent was evaporated and the residue was purified by preparative HPLC to afford the desired product as a brown oil (11 mg, 28% yield)

MS (ESI) m/z 350 (M+H). ¹H NMR (400 MHz, METHANOL-D3) δ ppm 7.76 (dd, J=7.69, 1.76 Hz, 1H), 7.51 (d, J=1.54 Hz, 1H), 7.44 (d, J=8.57 Hz, 1H), 7.27-7.31 (m, 1H), 7.06-7.13 (m, 3H), 3.98 (s, 3H), 3.66-3.74 (m, 2H), 3.50 (t, J=7.03 Hz, 2H), 2.80 (s, 3H), 2.51-2.58 (m, 1H), 2.30-2.40 (m, 1H).

### Example 137

### 2-(4-(2-(2-methoxyphenyl)-1H-indol-5-yl)-3,3-dimethylpiperidin-1-yl)-N-methylethanamine

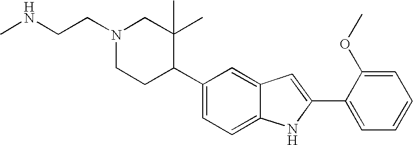 Part A: Tert-butyl 5,5-dimethyl4-(trifluoromethylsulfonyloxy)-5,6-dihydropyridine-1(2H)-carboxylate

To a solution of tert-butyl 3,3-dimethyl-4-oxopiperidine-1-carboxylate (prepared according to literature procedure J. Org. Chem., 2001, 66, 2487-2492) (454 mg, 2.0 mmol) in THF (15 ml) at −78° C. was added lithium bis(trimethylsilyl)amide (1 M in THF, 2.2 ml, 2.2 mmol) and the reaction solution was stirred for 1 h. A solution of N-(5-chloropyridin-2-yl)-1,1,1-trifluoro-N-(trifluoromethylsulfonyl) methanesulfonamide (864 mg, 2.2 mmol) in THF (20 ml) was added drop wise. The reaction mixture was stirred for another 0.5 h and allowed to warm to room temperature over 3 h and quenched with saturated sodium bicarbonate. The reaction mixture was diluted with ethyl acetate and washed with 15% potassium hydrogen sulfate, saturated sodium bicarbonate solution, 1 N sodium hydroxide, water and brine. The combined organic layers were dried over magnesium sulfate and concentrated in vacuo. Flash chromatography (silica gel, 10% ethyl acetate/hexane) gave the product as a colorless oil (604 mg, 84% yield)

Part B: Tert-butyl 4-(2-(2-methoxyphenyl)-1H-indol-5-yl)-5,5-dimethyl-5,6-dihydropyridine-1(2H)-carboxylate

A mixture of 2-(2-methoxyphenyl)-5-(4,4,5,5-tetramethyl-1,3,2-dioxaborolan-2-yl)-1H-indole (prepared using procedure provided for Part C of example 98) (209 mg, 0.60 mmol), tert-butyl 5,5-dimethyl-4-(trifluoromethylsulfonyloxy)-5,6-dihydropyridine-1(2H)-carboxylate (215 mg, 0.60 mmol), [1,1′-bis(diphenylphosphino) ferrocene]dichloropalladium (29 mg, 0.036 mmol) and Cesium carbonate (590 mg, 1.80 mmol) in DMF (3 ml) was heated at 100° C. under nitrogen overnight. The reaction mixture was partitioned between ethyl acetate and water, The organic layer was washed with saturated sodium bicarbonate (3 times), dried over magnesium sulfate and concentrated in vacuo, purification by flash column chromatography, eluting with 10% ethyl acetate/hexane, afforded the desired product as a brown oil (220 mg, 85% yield).

MS (ESI) m/z 433 (M+H).

Part C: Tert-butyl 2-(4-(2-(2-methoxyphenyl)-1H-indol-5-yl)-5,5-dimethyl-5,6-dihydropyridin-1(2H)-yl)ethyl(methyl)carbamate

Tert-butyl 4-(2-(2-methoxyphenyl)-1H-indol-5-yl)-5,5-dimethyl-5,6-dihydropyridine-1(2H)-carboxylate (220 mg, 0.51 mmol) was treated with 3 ml of 25% TFA in dichloroethane at room temperature for 1 h. Solvent was evaporated and the residue was purified by flush column chromatography, eluting with 3% 2N ammonia/methanol/dichloromethane to give the intermediate as a brown oil.

A mixture of the above intermediate, tert-butyl methyl(2-oxoethyl)carbamate (preparation as given in Part A of example 1) (88 mg, 0.51 mmol), sodium triacetoxyboronhydride (151 mg, 0.71 mmol), and acetic acid (31 mg, 0.51 mmol) in dichloroethane (20 ml) was stirred at room temperature overnight. The reaction mixture was diluted with ethyl acetate and washed 3 times with saturated sodium bicarbonate. The organic layer was dried over magnesium sulfate and concentrated in vacuo, the crude was used without purification (310 mg).

MS (ESI) m/z 490 (M+H).

Part D: 2-(4-(2-(2-methoxyphenyl)-1H-indol-5-yl)-3,3-dimethylpiperidin-1-yl)-N-methylethanamine

A mixture of crude tert-butyl 2-(4-(2-(2-methoxyphenyl)-1H-indol-5-yl)-5,5-dimethyl-5,6-dihydropyridin-1(2H)-yl)ethyl(methyl)carbamate (310 mg) and 10% palladium on carbon (67 mg) in methanol (30 ml) was hydrogenated at 30 psi overnight. The reaction mixture was filtered through a pad of Celite and the filtrate was concentrated in vacuo to give an oil which was subsequently treated with 25% TFA in dichloroethane (3 ml) room temperature for 1 h. Solvent was evaporated and the residue was purified by preparative HPLC to afford the desired product as a brown oil (122 mg, 39% yield).

MS (ESI) m/z 392 (M+H). ¹H NMR (400 MHz, METHANOL-D3) δ ppm 7.77 (dd, J=7.80, 1.65 Hz, 1H), 7.38 (d, J=8.35 Hz, 1H), 7.34 (s, 1H), 7.28 (td, J=7.80, 1.54 Hz, 1H), 7.10 (d, J=7.69 Hz, 1H), 7.02 (td, J=7.47, 1.10 Hz, 1H), 6.93 (dd, J=8.46, 1.65 Hz, 1H), 3.98 (s, 3H), 3.74-3.80 (m, 1H), 3.50-3.61 (m, 4H), 3.41 (dd, J=12.30, 1.76 Hz, 1H), 3.12 (td, J=12.58, 2.97 Hz, 1H), 3.04 (d, J=12.30 Hz, 1H), 2.76-2.83 (m, 4H), 2.56-2.69 (m, 1H), 1.89-1.96 (m, 1H), 1.07 (s, 3H), 0.93 (s, 3H).

### Example 138

### 2-(4-(2-(2-chlorophenyl)-3-fluoro-1H-indol-5-yl)piperidin-1-yl)-N-methylethanamine

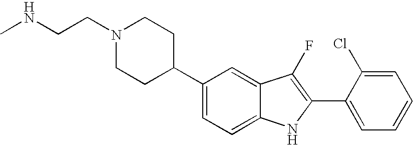 Part A: Preparation of tert-butyl 4-(4-amino-3-((2-chlorophenyl)ethynyl)phenyl)piperidine-1-carboxylate

A mixture of tert-butyl 4-(4-amino-3-iodophenyl)piperidine-1-carboxylate prepared using procedure provided for Part A of example 99, method 2) (200 mg, 0.5 mmol), 1-chloro-2-ethynylbenzene (75 mg, 0.55 mmol), dichlorobistriphenylphosphinepalladium(II) (7.0 mg, 0.01 mmol) and copper(I) iodide (1.0 mg, 0.005 mmol) in triethylamine (2 ml) was heated at 55° C. under nitrogen for 6 h. Solvent was evaporated and the crude was purified by column chromatography, eluting with 10% ethyl acetate/hexanes to give a light brown solid (142 mg, 69% yield).

MS (ESI) m/z 411 (M+H). ¹H NMR (400 MHz, CHLOROFORM-D) δ ppm 7.51-7.59 (m, 1H), 7.38-7.46 (m, 1H), 7.20-7.28 (m, 3H), 7.00 (dd, J=8.35, 2.20 Hz, 1H), 6.70 (d, J=8.35 Hz, 1H), 4.22(br. s, 2H), 2.77 (t, J=11.97 Hz, 2H), 2.48-2.57 (m, 1H), 1.78 (d, J=12.74 Hz, 2H), 1.58(td, J=12.63, 4.17 Hz, 2H), 1.47(s, 9H), 1.21-1.32(m, 1H), 0.81-0.90 (m, 1H)

Part B: Preparation of tert-butyl 4-(2-(2-chlorophenyl)-1H-indol-5-yl)piperidine-1-carboxylate

A mixture of tert-butyl 4-(4-amino-3-((2-chlorophenyl)ethynyl)phenyl)piperidine-1-carboxylate (142 mg, 0.35 mmol) and palladium chloride (3.1 mg, 0.017 mmol) in acetonitrile (3 ml) was heated at 75° C. overnight. Solvent was evaporated and the residual was purified by column chromatography, eluting with 30% ethyl acetate/hexanes (120 mg, 85% yield).

MS (ESI) m/z 411 (M+H).

Part C: Preparation of tert-butyl 2-(4-(2-(2-chlorophenyl)-1H-indol-5-yl)piperidin-1-yl)ethyl(methyl)carbamate

Tert-butyl 4-(2-(2-chlorophenyl)-1H-indol-5-yl)piperidine-1-carboxylate (68 mg, 0.17 mmol) was treated with 25% TFA in dichloroethane (2 ml) at room temperature for 1 h. solvent was evaporated and the residue was purified by column chromatography, eluting with 8% 2N ammonia/methanol/dichloromethane. A mixture of the above intermediate, tert-butyl methyl(2-oxoethyl)carbamate (34 mg, 0.20 mmol), sodium triacetoxyboronhydride (49 mg, 0.23 mmol), and acetic acid (10 mg, 0.17 mmol) in dichloromethane (4 ml) was stirred at room temperature overnight. The reaction mixture was diluted with ethyl acetate and washed 3 times with saturated sodium bicarbonate. The organic layer was dried over magnesium sulfate and concentrated in vacuo to afford the crude product (86 mg) which was used without purification.

MS (ESI) m/z 468 (M+H).

Part D: Preparation of tert-butyl 2-(4-(2-(2-chlorophenyl)-3-fluoro-1H-indol-5-yl)piperidin-1-yl)ethyl(methyl)carbamate

Tert-butyl 2-(4-(2-(2-chlorophenyl)-1H-indol-5-yl)piperidin-1-yl)ethyl(methyl)carbamate (86 mg, 0.18 mmol) was dissolved in a 1 to 1 mixture of methyl sulfoxide and acetonitrile (4 ml). The solution was cooled to 0° C. and the fluorinating reagent, 1-(chloromethyl)-4-fluoro-1,4-diazoniabicyclo[2.2.2]octane ditetrafluoroborate (SelectFluor, 0.25 equiv., 16 mg, 0.046 mmol) was added. After addition the cooling bath was removed and the reaction mixture was stirred at room temperature for 30 minutes. The fluorinating reagent was continually added in this fashion until half of the starting material was consumed (1.0 equivalent of SelectFluor was added). The reaction mixture was partitioned between ethyl acetate and water and the two layers were separated. The organic layer was washed with saturated sodium bicarbonate, dried over magnesium sulfate and concentrated in vacuo, the crude product was purified by preparative HPLC (45-80% gradient) to afford the desired product (60 mg, 56% yield).

MS (ESI) m/z 485 (M+H).

Part E: Preparation of 2-(4-(2-(2-chlorophenyl)-3-fluoro-1H-indol-5-yl)piperidin-1-yl)-N-methylethanamine

Tert-butyl 2-(4-(2-(2-chlorophenyl)-3-fluoro-1H-indol-5-yl)piperidin-1-yl)ethyl(methyl)carbamate (60 mg, 0.10 mmol) was treated with 25% TFA in dichloroethane (2 ml) at room temperature for 0.5 h, solvent was evaporated and the residue was purified by preparative HPLC to afford the final product (38 mg, 62% yield).

MS (ESI) m/z 386 (M+H). ¹H NMR (400 MHz, METHANOL-D3) δ ppm 7.27-7.66 (m, 6H), 7.10 (ddd, J=22.58, 8.51, 1.65 Hz, 1H), 3.72-3.78 (m, 2H), 3.51-3.57 (m, 4H), 3.18-3.32 (m, 2H), 2.95-3.03 (m, 1H), 2.82 (s, 3H), 2.13-2.21 (m, 4H).

### Example 139

### N-(4-fluoro-2-(3-fluoro-5-(1-(2-(methylamino)ethyl) piperidin-4-yl)-1H-indol-2-yl)phenyl)methanesulfonamide

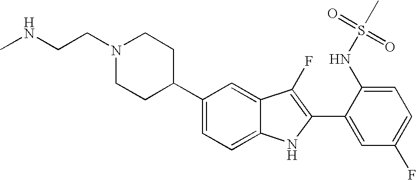 Part A: Preparation of tert-butyl 4-(4-amino-3-((2-(methylsulfonamido)phenyl)ethynyl)phenyl)piperidine-1-carboxylate

A mixture of tert-butyl 4-(4-amino-3-iodophenyl)piperidine-1-carboxylate (prepared using procedure provided for Part A of example 99, method 2) (200 mg, 0.5 mmol), N-(2-ethynylphenyl)methanesulfonamide (107 mg, 0.55 mmol), dichlorobistriphenylphosphinepalladium(II) (7.0 mg, 0.01 mmol) and copper(I) iodide (1.0 mg, 0.005 mmol) in triethylamine (2 ml) was heated at 55° C. under nitrogen for 6 h. Solvent was evaporated and the crude was purified by column chromatography, eluting with 10% ethyl acetate/hexanes to give a light brown solid (180 mg, 77% yield).

MS (ESI) m/z 470 (M+H).

Part B: Preparation of tert-butyl 4-(2-(2-(methylsulfonamido)phenyl)-1H-indol-5-yl)piperidine-1-carboxylate

Potassium tert-butoxide (95%, 59 mg, 0.50 mmol) was added to a solution of tert-butyl 4-(4-amino-3-((2-(methylsulfonamido)phenyl)ethynyl)phenyl)piperidine-1-carboxylate (180 mg, 0.38 mmol) in NMP at room temperature and the resulting mixture was stirred for 2 h. The reaction mixture was partitioned between ethyl acetate and water and the two layers were separated. The organic layer was washed with saturated sodium bicarbonate, dried over magnesium sulfate and concentrated in vacuo, the residual was purified by column chromatography, eluting with 30% ethyl acetate/hexanes (124 mg, 69% yield).

MS (ESI) m/z 470 (M+H).

Part C: Preparation of tert-butyl methyl(2-(4-(2-(2-(methylsulfonamido)phenyl)-1H-indol-5-yl)piperidin-1-yl)ethyl)carbamate

Tert-butyl 4-(2-(2-(methylsulfonamido)phenyl)-1H-indol-5-yl)piperidine-1-carboxylate (124 mg, 0.26 mmol) was treated with 25% TFA in dichloroethane (2 ml) at room temperature for 1 h. solvent was evaporated and the residue was purified by column chromatography, eluting with 8% 2N ammonia/methanol/dichloromethane.

A mixture of the above intermediate, tert-butyl methyl(2-oxoethyl)carbamate (55 mg, 0.32 mmol), sodium triacetoxyboronhydride (78 mg, 0.37 mmol), and acetic acid (16 mg, 0.26 mmol) in dichloromethane (4 ml) was stirred at room temperature overnight. The reaction mixture was diluted with ethyl acetate and washed 3 times with saturated sodium bicarbonate. The organic layer was dried over magnesium sulfate and concentrated in vacuo. Purification by flash column chromatography, eluting with 3% 2N ammonia/methanol/dichloromethane afforded the desired product (68 mg, 49% yield).

MS (ESI) m/z 527 (M+H).

Part D: Preparation of tert-butyl 2-(4-(3-fluoro-2-(2-(methylsulfonamido)phenyl)-1H-indol-5-yl)piperidin-1-yl)ethyl(methyl)carbamate

Tert-butyl methyl(2-(4-(2-(2-(methylsulfonamido)phenyl)-1H-indol-5-yl)piperidin-1-yl)ethyl)carbamate (68 mg, 0.13 mmol) was dissolved in a 1 to 1 mixture of methyl sulfoxide and acetonitrile (4 ml). The solution was cooled to 0° C. and the fluorinating reagent, 1-(chloromethyl)-4-fluoro-1,4-diazoniabicyclo[2.2.2]octane ditetrafluoroborate (SelectFluor, 0.25 equiv., 11 mg, 0.032 mmol) was added. After addition the cooling bath was removed and the reaction mixture was stirred at room temperature for 30 minutes. The fluorinating reagent was continually added in this fashion until half of the starting material was consumed (1.0 equivalent of SelectFluor was added). The reaction mixture was partitioned between ethyl acetate and water and the two layers were separated. The organic layer was washed with saturated sodium bicarbonate, dried over magnesium sulfate and concentrated in vacuo, the crude product was purified by preparative HPLC (45-80% gradient) to afford the desired product (14 mg, 20% yield).

MS (ESI) m/z 545 (M+H).

Part E: Preparation of N-(2-(3-fluoro-5-(1-(2-(methylamino)ethyl)piperidin-4-yl)-1H-indol-2-yl)phenyl)methanesulfonamide

Tert-butyl 2-(4-(3-fluoro-2-(2-(methylsulfonamido)phenyl)-1H-indol-5-yl)piperidin-1-yl)ethyl(methyl)carbamate (14 mg, 0.021 mmol) was treated with 25% TFA in dichloroethane (2 ml) at room temperature for 0.5 h, solvent was evaporated and the residue was purified by preparative HPLC to afford the final product (6 mg, 42% yield).

MS (ESI) m/z 445 (M+H). ¹H NMR (400 MHz, METHANOL-D3) δ ppm 7.53-7.59 (m, 3H), 7.36-7.40 (m, 2H), 7.17-7.23 (m, 1H), 7.07-7.14 (m, 1 Hz, 1H), 3.72-3.76 (m, 2H), 3.53 (s, 4H), 3.18-3.31 (m, 2H), 2.95-3.05 (m, 1H), 2.81 (s, 3H), 2.80 (s, 3H), 2.10-2.22 (m, 4H).

### Example 140

### 2-(4-(3-fluoro-2-(5-fluoro-2-methoxyphenyl)-1H-indol-5-yl)piperidin-1-yl)-N-methylethanamine

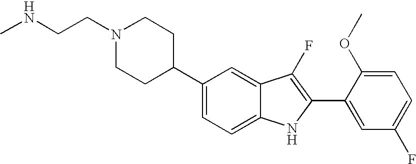 Part A: Preparation of tert-butyl 4-(4-amino-3-((5-fluoro-2-methoxyphenyl)ethynyl)phenyl)piperidine-1-carboxylate

A mixture of tert-butyl 4-(4-amino-3-iodophenyl)piperidine-1-carboxylate (prepared using procedure provided for Part A of example 99, method 2) (201 mg, 0.50 mmol), 2-ethynyl-4-fluoro-1-methoxybenzene (83 mg, 0.55 mmol), dichlorobistriphenylphosphinepalladium(II) (7.0 mg, 0.01 mmol) and copper(I) iodide (1.0 mg, 0.005 mmol) in triethylamine (2 ml) was heated at 55° C. under nitrogen for 6 h. Solvent was evaporated and the crude was purified by column chromatography, eluting with 10% ethyl acetate/hexanes to give a light brown solid (210 mg, 99% yield).

MS (ESI) m/z 425 (M+H).

Part B: Preparation of tert-butyl 4-(2-(5-fluoro-2-methoxyphenyl)-1H-indol-5-yl)piperidine-1-carboxylate

A mixture of tert-butyl 4-(4-amino-3-((5-fluoro-2-methoxyphenyl)ethynyl)phenyl)piperidine-1-carboxylate (210 mg, 0.50 mmol) and palladium chloride (4.4 mg, 0.025 mmol) in acetonitrile (5 ml) was heated at 75° C. overnight. Solvent was evaporated and the residual was purified by column chromatography, eluting with 20% ethyl acetate/hexanes (200 mg, 95% yield).

MS (ESI) m/z 425 (M+H).

Part C: Preparation of tert-butyl 2-(4-(2-(5-fluoro-2-methoxyphenyl)-1H-indol-5-yl)piperidin-1-yl)ethyl(methyl)carbamate

Tert-butyl 4-(2-(5-fluoro-2-methoxyphenyl)-1H-indol-5-yl)piperidine-1-carboxylate (200 mg, 0.47 mmol) was treated with 25% TFA in dichloroethane (3 ml) at room temperature for 1 h. solvent was evaporated and the residue was purified by column chromatography, eluting with 8% 2N ammonia/methanol/dichloromethane.

A mixture of the above intermediate, tert-butyl methyl(2-oxoethyl)carbamate (90 mg, 0.52 mmol), sodium triacetoxyboronhydride (140 mg, 0.66 mmol), and acetic acid (31 mg, 0.52 mmol) in dichloromethane (4 ml) was stirred at room temperature overnight. The reaction mixture was diluted with ethyl acetate and washed 3 times with saturated sodium bicarbonate. The organic layer was dried over magnesium sulfate and concentrated in vacuo. Purification by flash column chromatography, eluting with 3% 2N ammonia/methanol/dichloromethane afforded the desired product (144 mg, 63% yield).

MS (ESI) m/z 482 (M+H).

Part D: Preparation of tert-butyl 2-(4-(3-fluoro-2-(5-fluoro-2-methoxyphenyl)-1H-indol-5-yl)piperidin-1-yl)ethyl(methyl)carbamate

Tert-butyl 2-(4-(2-(5-fluoro-2-methoxyphenyl)-1H-indol-5-yl)piperidin-1-yl)ethyl(methyl)carbamate (144 mg, 0.30 mmol) was dissolved in a 1 to 1 mixture of methyl sulfoxide and acetonitrile (10 ml). The solution was cooled to 0° C. and the fluorinating reagent, 1-(chloromethyl)-4-fluoro-1,4-diazoniabicyclo[2.2.2]octane ditetrafluoroborate (SelectFluor, 0.25 equiv., 53 mg, 0.15 mmol) was added. After addition the cooling bath was removed and the reaction mixture was stirred at room temperature for 30 minutes. The fluorinating reagent was continually added in this fashion until half of the starting material was consumed (2.0 equivalent of SelectFluor was added). The reaction mixture was partitioned between ethyl acetate and water and the two layers were separated. The organic layer was washed with saturated sodium bicarbonate, dried over magnesium sulfate and concentrated in vacuo, the crude product was purified by preparative HPLC (40-80% gradient) to afford the desired product (40 mg, 27% yield).

MS (ESI) m/z 500 (M+H).

Part E: Preparation of 2-(4-(3-fluoro-2-(5-fluoro-2-methoxyphenyl)-1H-indol-5-yl)piperidin-1-yl)-N-methylethanamine

Tert-butyl 2-(4-(3-fluoro-2-(5-fluoro-2-methoxyphenyl)-1H-indol-5-yl)piperidin-1-yl)ethyl(methyl)carbamate (40 mg, 0.080 mmol) was treated with 25% TFA in dichloroethane (3 ml) at room temperature for 1 h, solvent was evaporated and the residue was purified by preparative HPLC to afford the final product (20 mg, 63% yield).

MS (ESI) m/z 400 (M+H). ¹H NMR (400 MHz, METHANOL-D3) δ ppm 7.55 (dd, J=9.89, 3.08 Hz, 1H), 7.42 (s, 1H), 7.40 (dd, J=8.57, 2.42 Hz, 1H), 7.09-7.14 (m, 2H), 7.04 (ddd, J=9.12, 7.80, 3.08 Hz, 1H), 3.95 (s, 3H), 3.68-3.76 (m, 2H), 3.47-3.56 (m, 4H), 3.16-3.24(m, 2H), 2.94-3.03 (m, 1H), 2.82 (s, 3H), 2.10-2.20(m, 4H).

### Example 141

### 2-(2-methoxyphenyl)-5-(1-(pyrrolidin-3-yl)piperidin-4-yl)-1H-indole

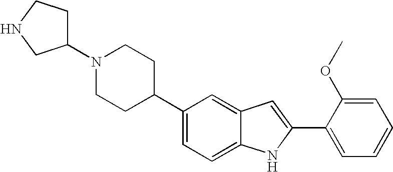

A mixture of 2-(2-methoxyphenyl)-5-(piperidin-4-yl)-1H-indole (preparation as given in Part D of example 99, method 2) (18 mg, 0.059 mmol), tert-butyl 3-oxopyrrolidine-1-carboxylate (11 mg, 0.059 mmol), sodium triacetoxyboronhydride (18 mg, 0.082 mmol), acetic acid (3.5 mg, 0.059 mmol) and 4 A molecular sieves (1 g) in dichloromethane (10 ml) was stirred at room temperature overnight. The reaction mixture was filtered and the filtrate was washed 3 times with saturated sodium bicarbonate. The organic layer was dried over magnesium sulfate and concentrated in vacuo. The crude intermediate was treated with 25% TFA in dichloroethane (1 ml) at room temperature for 1 h, solvent was evaporated and the residue was purified by preparative HPLC to afford the final product (3 mg, 71% yield).

MS (ESI) m/z 376 (M+H). ¹H NMR (400 MHz, METHANOL-D3) δ ppm 7.76 (dd, J=7.69, 1.54 Hz, 1H), 7.41 (s, 1H), 7.40 (d, J=8.57 Hz, 1H), 7.25-7.31 (m, 1H), 7.10-7.13 (m, 1H), 6.99-7.04 (m, 2H), 4.03-4.15 (m, 1H), 3.98 (s, 3H), 373-3.88 (m, 2H), 3.59-3.70 (m, 3H), 3.32-3.40 (m, 1H), 3.15-3.28 (m, 2H), 2.88-3.03 (m, 1H), 2.55-2.72 (m, 1H), 2.24-2.42 (m, 1H), 2.05-2.20 (m, 4H).

### Example 142

### 3-(4-(2-(2-methoxyphenyl)-1H-indol-5-yl)piperidin-1-yl)-N-methylpropan-1-amine

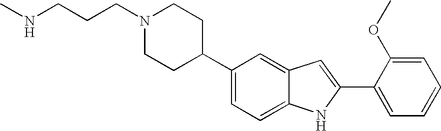 Part A: Preparation of tert-butyl 3-(4-(2-(2-methoxyphenyl)-1H-indol-5-yl)piperidin-1-yl)propylcarbamate

A mixture of 2-(2-methoxyphenyl)-5-(piperidin-4-yl)-1H-indole (preparation as given in Part D of example 99, method 2) (80 mg, 0.26 mmol), tert-butyl methyl(3-oxopropyl)carbamate (prepared according to literature Tetrahedron, 2002, 58, 1719-1737) (45 mg, 0.26 mmol), sodium triacetoxyboronhydride (78 mg, 0.37 mmol), and acetic acid (16 mg, 0.26 mmol) in dichloromethane (10 ml) was stirred at room temperature overnight. The reaction mixture was diluted with ethyl acetate and washed 3 times with saturated sodium bicarbonate. The organic layer was dried over magnesium sulfate and concentrated in vacuo, the crude was purified by flash column chromatography, eluting with 3% 2N ammonia/methanol/dichloromethane to give the desired product (72 mg, 60% yield).

MS (ESI) m/z 464 (M+H).

Part B: Preparation of 3-(4-(2-(2-methoxyphenyl)-1H-indol-5-yl)piperidin-1-yl)-N-methylpropan-1-amine

To a solution of tert-butyl 3-(4-(2-(2-methoxyphenyl)-1H-indol-5-yl)piperidin-1-yl)propylcarbamate (72 mg, 0.16 mmol) in THF (3 ml) at room temperature was added lithium aluminum hydride (31 mg, 0.80 mmol) and the resulting mixture was heated at reflux for 2 hours. The reaction mixture was cooled to room temperature and 0.15 ml of water, 0.15 ml of 20% sodium hydroxide and 0.15 ml of water were added drop wise subsequently. The resulting mixture was filtered through a pad of Celite and the filtrate was concentrated in vacuo. The crude was purified by preparative HPLC to afford the desired product (47 mg, 78% yield).

MS (ESI) m/z 378 (M+H). ¹H NMR (400 MHz, METHANOL-D3) δ ppm 7.76 (dd, J=7.80, 1.65 Hz, 1H), 7.37-7.42 (m, 2H), 7.28 (td, J=7.85, 1.65 Hz, 1H), 7.11 (d, J=8.35 Hz, 1H), 6.98-7.04 (m, 2H), 3.98 (s, 3H), 3.66-3.70 (m, 2H), 3.20-3.28 (m, 2H), 3.07-3.18 (m, 4H), 2.89-2.97 (m, 1H), 2.74 (s, 3H), 2.02-2.24 (m, 6H).

### Example 143

### 2-(6-(2-(2-methoxyphenyl)-1H-indol-5-yl)-3-azabicyclo[4.1.0]heptan-3-yl)-N-methylethanamine

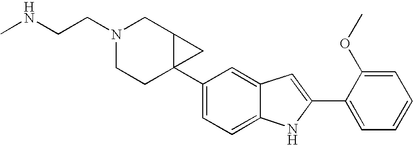

TFA (34 mg, 0.3 mmol) in dichloromethane (0.5 ml) was added slowly to a solution of diethyl zinc (1M solution in DCM, 0.3 ml, 0.3 mmol) in dichloromethane (1 ml) under nitrogen at 0° C. and the solution was stirred for 20 minutes. A solution of diiodomethane (79 mg, 0.3 mmol) in dichloromethane (0.5 ml) was then added and the reaction solution stirred for another 20 minutes before tert-butyl 2-(4-(2-(2-methoxyphenyl)-1H-indol-5-yl)-5,6-dihydropyridin-1(2H)-yl)ethyl(methyl)carbamate (preparation as given in Part E of example 98) 68 mg, 0.15 mmol) in dichloromethane (1 ml) was added. The cooling bath was removed and the reaction mixture stirred for 30 minutes and was quenched with aqueous ammonium chloride followed by addition of saturated sodium bicarbonate. The reaction mixture was extracted with ethyl acetate 3 times. The combined organic layers were dried over magnesium sulfate and concentrated in vacuo. The crude was purified by preparative HPLC to give the intermediate as a brown oil (20 mg) which was then treated with 25% TFA in dichloroethane (1 ml) at room temperature for 1 h. TFA was evaporated and the residue was again purified by preparative HPLC to afford the desired product (2.8 mg, 5% yield).

MS (ESI) m/z 376 (M+H). ¹H NMR (400 MHz, METHANOL-D3) δ ppm 7.58 (d, J=1.54 Hz, 1H), 7.23-7.42 (m, 5H), 7.11 (d, J=8.35 Hz, 1H), 7.04(td, J=7.47, 0.88 Hz, 1H), 6.06-6.108 (m, 1H), 3.86-3.92 (m, 2H), 3.84 (s, 3H), 3.44-3.55 (m, 4H), 2.96-3.02 (m, 2H), 2.77-2.87 (m, 5H), 2.26-2.30 (m, 3H).

### Example 145

### 2-(4-(2-(2-methoxyphenyl)-3,3-dimethylindolin-5-yl)piperidin-1-yl)-N-methylethanamine

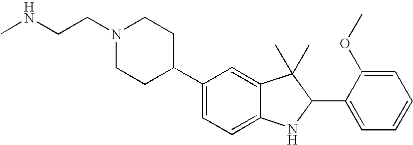 Part A: Preparation of 1-(2-methoxyphenyl)-2-methylpropan-1-one

Prepared using the procedure reported by Weiberth, Franz J et al (Journal of Organic Chemistry (1987), 52(17), 3901-4)

Part B: Preparation of 5-bromo-2-(2-methoxyphenyl)-3,3-dimethylindoline

To a solution of 4-bromophenyl hydrazine hydrochloride (2.5 g, 11.2 mmol) and 1-(2-methoxyphenyl)-2-methylpropan-1-one (2.39 g, 13.44 mmol) in 20 mL of toluene was added acetic acid (cat) and the resulting solution was refluxed with azeotropic removal of water for 16 hours. The reaction mixture was cooled to room temperature and concentrated. The resulting semi-solid residue was taken up in acetic acid and heated to reflux for 12 hours. The resulting dark solution was cooled and concentrated in vaccuo. The semi-solid residue was taken up in diethyl ether and netralized with solid potassium carbonate. The ether layer was concentrated. The resulting imine was dissolved in a mixture of THF-methanol (6: 1; 20 M1), cooled (0° c.) and sodium borohydride (0.53 g, 13.2 mmol) was added. The solution was allowed to warm to room temperature over a period of 45 minutes. The reaction mixture was diluted with 20 M1 of ethyl acetate, poured into 20 M1 OF 1 N aqueous HCl and the resulting solution was gradually basified using potassium carbonate. The organic layer was separated, washed with brine, dried (Na2SO4)and concentarted. The crude product was purified using silica gel chromatography (3:1 ethyl acetate-hexane) to afford 1.9 g of 5-bromo-2-(2-methoxyphenyl)-3,3-dimethylindoline

1H NMR (400 MHz, METHANOL-D3) δ ppm 7.2 (m, 1H), 7.04 (d, 1H), 6.80-6.84 (m, 4H), 6.01 (d, 1H), 3.87 (s, 3H), 3.77(s, 1H), 1.53 (s, 3H), 1.07 (s, 3H) MS (ESI) m/z 332 (M+H).

Part C: Preparation of 2-(2-methoxyphenyl)-3,3-dimethyl-5-(4,4,5,5-tetramethyl-1,3,2-dioxaborolan-2-yl)indoline

A mixture of 5-bromo-2-(2-methoxyphenyl)-3,3-dimethylindoline (420 mg, 1.3 mmol), bis(pinacolato)diboron (482 mg, 1.9 mmol), [1,1′-bis(diphenylphosphino)ferrocene]dichloropalladium (52 mg, 0.06 mmol) and potassium acetate (372 mg, 3.8 mmol) in methyl sulfoxide (6 ml) was heated at 90° C. under N₂ overnight, the reaction mixture was partitioned between ethyl acetate and water. The organic layer was washed 3 times with saturated sodium bicarbonate, dried over magnesium sulfate and concentrated in vacuo, flush column chromatography (eluting with 10% ethyl acetate/hexanes) gave a light brown solid (303 mg, 61% yield).

Part D: Preparation of tert-butyl 2-(4-(2-(2-methoxyphenyl)-3,3-dimethylindolin-5-yl)-5,6-dihydropyridin-1(2H)-yl)ethyl(methyl)carbamate

A mixture of 1-(2-(tert-butoxycarbonyl(methyl)amino)ethyl)-1,2,3,6-tetrahydropyridin-4-yl trifluoromethanesulfonate (prepared using procedure provided for Part C of example 99, method 3) (310 mg, 0.80 mmol), 2-(2-methoxyphenyl)-3,3-dimethyl-5-(4,4,5,5-tetramethyl-1,3,2-dioxaborolan-2-yl)indoline (303 mg, 0.80 mmol), [1,1′-bis(diphenylphosphino) ferrocene]dichloropalladium (39 mg, 0.048 mmol) and Cesium carbonate (782 g, 2.4 mmol) in DMF (4 ml) was heated at 100° C. under N₂ overnight. The reaction mixture was partitioned between ethyl acetate and water, The organic layer was washed with saturated sodium bicarbonate (3 times), dried over magnesium sulfate and concentrated in vacuo, purification by flash column chromatography, eluting with 3% ammonia/methanol/dichloromethane, afforded the desired product (39 mg, 10% yield).

MS (ESI) m/z 492 (M+H).

Part D: Preparation of 2-(4-(2-(2-methoxyphenyl)-3,3-dimethylindolin-5-yl)piperidin-1-yl)-N-methylethanamine

A mixture of tert-butyl 2-(4-(2-(2-methoxyphenyl)-3,3-dimethylindolin-5-yl)-5,6-dihydropyridin-1(2H)-yl)ethyl(methyl)carbamate (39 mg, 0.079 mmol) and 10% palladium on carbon (10 mg) in methanol (20 ml) was hydrogenated at 50 psi overnight. The reaction mixture was filtered through a pad of Celite and the filtrate was concentrated in vacuo to give an oil which was subsequently treated with 25% TFA in dichloroethane (2 ml) at room temperature for 1 h. TFA was evaporated and the residue was purified by preparative HPLC to afford the desired product as a brown oil (7.0 mg, 23% yield)

MS (ESI) m/z 394 (M+H). 1H NMR (400 MHz, METHANOL-D3) 8 ppm 7.44-7.50 (m, 1H), 7.32-7.42 (m, 4H), 7.17 (d, J=8.35 Hz, 1H), 7.06 (dt, J=7.47, 0.88 Hz 1H), 5.25 (s, 1H), 3.87 (s, 3H), 3.77-3.843 (m, 2H), 3.61 (br. s, 4H), 3.21-3.32 (m, 2H), 3.00-3.12 (m, 1H), 2.87 (s, 3H), 2.16-2.28 (m, 4H), 1.53 (s, 3H), 1.07 (s, 3H).

We claim:

1. A compound of formula I, or a stereoisomer, a tautomer, or a pharmaceutically acceptable salt thereof, 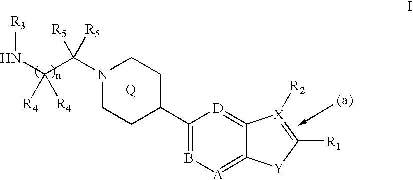
wherein:
Ring Q is 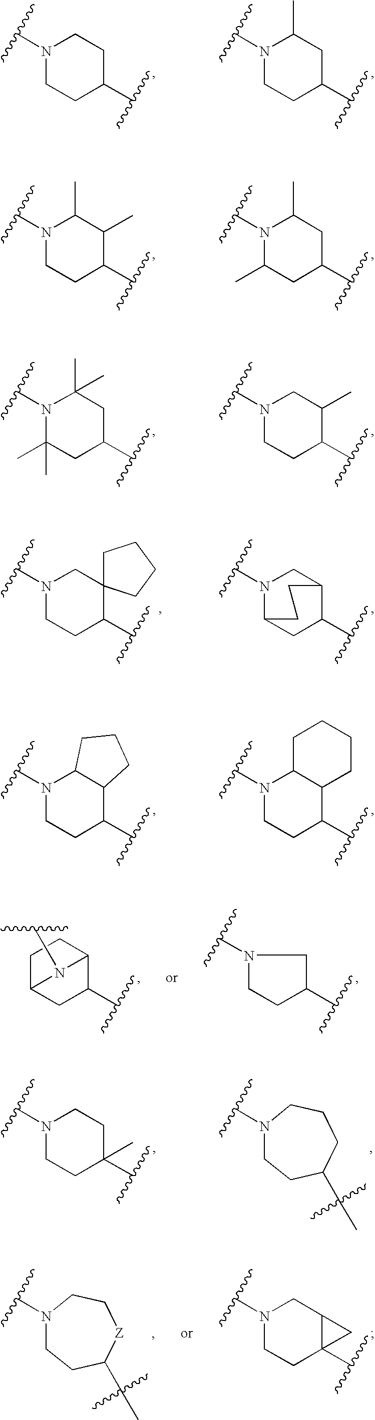
bond (a) is an optional double or single bond;
X is C (i.e., carbon) or N (i.e., nitrogen);
Y is NH, N-Me, or CH;
Z is N-R₆, O, or S, where R₆ is C₁-C₆ alkyl; wherein when bond (a) is a single bond, X is —CR—, R is indenpendently H or C₁₋₄ alkyl and CR₂ is H or C₁₋₄ alkyl; alternatively, R₂ and R may join to form a 3-6 membered cycloalkyl ring;
A, B and D are each independently N or C, in which C may be optionally substituted with H, Me, Et, halogen, CN, NO₂, OMe, OEt, SMe, SO₂Me, CF₃, or OCF₃;
R₁ is aryl, substituted aryl, aryalkyl, heterocycle, or substituted heterocycle;
R₂ is H, Me, Et, halogen, CN, NO₂, OMe, OEt, SMe, SO₂Me, CF₃, or OCF₃, provided that when X is N, R₂ is nil;
R₃ is H or C₁-C₄ alkyl; and
R₄ is independently H or C₁₋₄ alkyl;
R₅ is independently H, C₁₋₄ alkyl; alternatively, R₅ and R₃ may join to form a 4, 5, or 6 membered saturated ring containing one N; and
n is 1, 2, or 3.

2. A compound of claim 1, wherein the compound is of formula Id 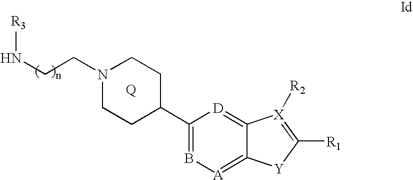
wherein:
Ring Q is 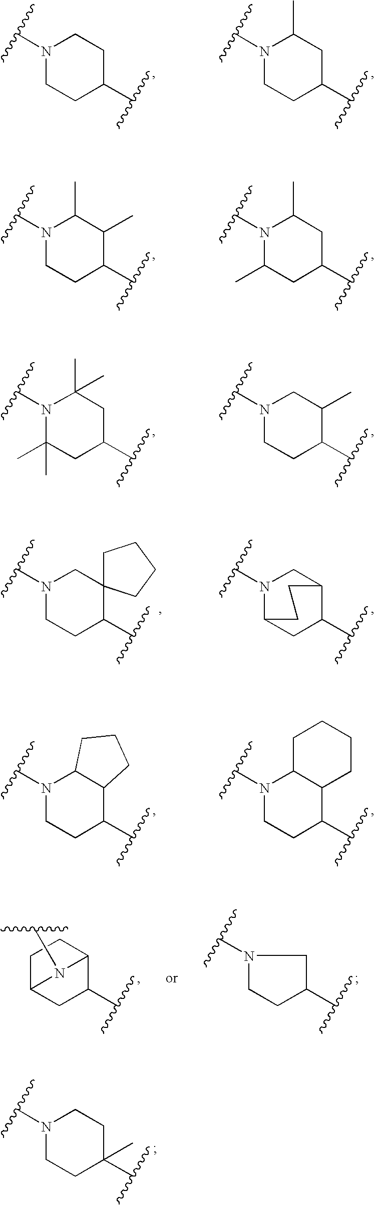
X is C (i.e., carbon) or N (i.e., nitrogen);
Y is NH, N-Me, or CH;
A, B and D are each independently N or C, in which C may be optionally substituted with H, Me, Et, halogen, CN, NO₂, OMe, OEt, SMe, SO₂Me, CF₃, or OCF₃;
R₁ is aryl, substituted aryl, aryalkyl, heterocycle, or substituted heterocycle;
R₂ is H, Me, Et, halogen, CN, NO₂, OMe, OEt, SMe, SO₂Me, CF₃, or OCF₃, provided that when X is N, R₂ is nil;
R₃ is H or C₁-C₄ alkyl; and
R₄ is independently H or C₁₋₄ alkyl; and
n is 1, 2, or 3.

3. The compound of claim 1, wherein ring Q is

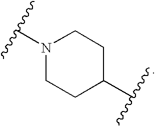

4. The compound of claim 3, wherein A, B and D are each independently C, which may be optionally substituted with H, Me, Et, halogen, CN, NO₂, OMe, OEt, SMe, SO₂Me, CF₃, or OCF₃.

5. The compound of claim 3, wherein R₁ is aryl or substituted aryl.

6. The compound of claim 3, wherein R₁ is heteroaryl or substituted heteroaryl.

7. The compound of claim 3, wherein A, B and D are each independently C, which may be optionally substituted with H, Me, Et, halogen, CN, NO₂, OMe, OEt, SMe, SO₂Me, CF₃, or OCF₃.

8. The compound of claim 7, wherein R₁ is aryl, substituted aryl, heteroaryl or substituted heteroaryl.

9. The compound of claim 8, wherein n is 1.

10. The compound of claim 9, wherein R₃ is Me.

11. The compound of claim 1 having the following substructure Ia, 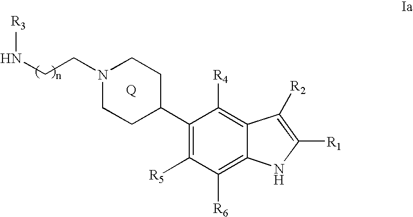
wherein:
Ring Q is 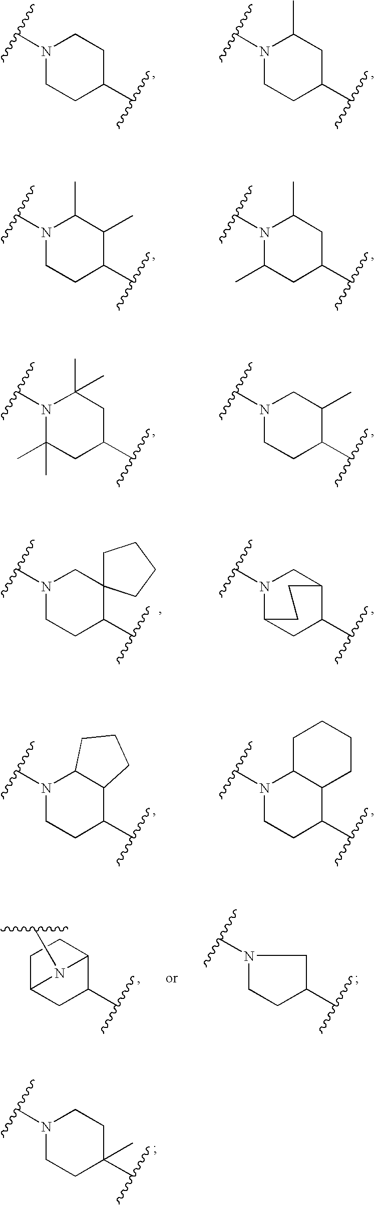
R₁ is aryl, substituted aryl, heterocycle, or substituted heterocycle;
R₃ is H or C₁-C₄ alkyl;
R₂, R₄, R₅ and R₆ are each independently H, Me, Et, halogen, CN, NO₂, OMe, OEt, SMe, SO₂Me, CF₃, or OCF3; and
n is 1, 2, or 3.

12. The compound of claim 1 having the following substructure Ib, 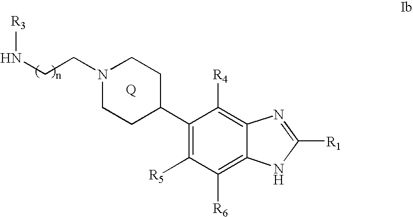
wherein:
Ring Q is 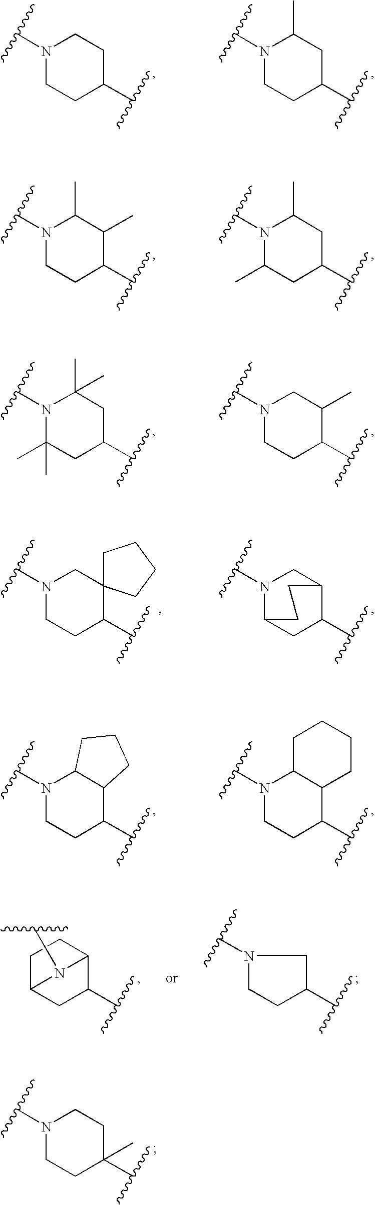
R₁ is aryl, substituted aryl, heterocycle, or substituted heterocycle;
R₃ is H or C₁-C₄ alkyl;
R₄, R₅ and R₆ are each independently H, Me, Et, halogen, CN, NO₂, OMe, OEt, SMe, SO₂Me, CF₃, or OCF3; and
n is 1, 2, or 3.

13. The compound of claim 12, wherein ring Q is

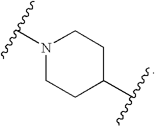

14. The compound of claim 13, wherein R₁ is aryl, substituted aryl, heteroaryl or substituted heteroaryl.

15. The compound of claim 14, wherein n is 1.

16. The compound of claim 14, wherein R₄ and R₅ are each independently H.

17. The compound of claim 16, wherein R₆ is H or Me.

18. The compound of claim 17, wherein R₃ is Me.

19. A pharmaceutical composition comprising at least one compound according to claim 1 and a pharmaceutically-acceptable carrier or diluent.

20. A method for treating a condition or disorder comprising administering to a mammalian species in need thereof a therapeutically effective amount of at least one compound of claim 1, or a stereoisomer, a tautomer, a pharmaceutically acceptable salt or solvate thereof,
wherein said condition or disorder is selected from the group consisting of proliferate diseases, cancers, benign prostate hypertrophia, benign prostatic hyperplasia, adenomas and neoplasies of the prostate, benign or malignant tumor cells containing the androgen receptor, brain cancer, skin cancer, bladder cancer, lymphatic cancer, liver cancer, kidney cancer, pancreatic cancer, prostate cancer, hirsutism, acne, precocious puberty, angiogenic conditions or disorders, hyperpilosity, inflammation, immune modulation, seborrhea, endometriosis, polycystic ovary syndrome, androgenic alopecia, hypogonadism, osteoporosis, suppressing spermatogenesis, male and female sexual dysfunction, libido, cachexia, anorexia, inhibition of muscular atrophy in ambulatory patients, androgen supplementation for age related decreased testosterone levels in men, cancers expressing the estrogen receptor, breast cancer, ovarian cancer, uterine cancer, endometrial cancer, hot flushes, vaginal dryness, menopause, amennoreahea, dysmennoreahea, contraception, pregnancy termination, cancers containing the progesterone receptor, cyclesynchrony, meniginoma, fibroids, labor induction, autoimmune diseases, Alzheimer's disease, psychotic disorders, drug dependence, non-insulin dependent Diabetes Mellitus, dopamine receptor mediated disorders, heart disease, congestive heart failure, disregulation of cholesterol homeostasis, and attenuating the metabolism of a pharmaceutical agent.
